(19) United States
(12) Patent Application Publication
(10) Pub. No.: US 2023/0384676 A1
(43) Pub. Date: Nov. 30, 2023

(54) RADIATION-SENSITIVE RESIN COMPOSITION, METHOD OF FORMING PATTERN, POLYMER, AND COMPOUND

(71) Applicant: JSR CORPORATION, Tokyo (JP)

(72) Inventors: Takuya OMIYA, Tokyo (JP); Katsuaki NISHIKORI, Tokyo (JP); Kazuya KIRIYAMA, Tokyo (JP); Yuushi MATSUMURA, Tokyo (JP); Natsuko KINOSHITA, Tokyo (JP)

(73) Assignee: JSR CORPORATION, Tokyo (JP)

(21) Appl. No.: 18/135,838

(22) Filed: Apr. 18, 2023

(30) Foreign Application Priority Data
May 25, 2022 (JP) 2022-085317

(51) Int. Cl. G03F 7/039; C08F 136/16; C07C 69/653

(52) U.S. Cl. CPC G03F 7/0397; C08F 136/16; C07C 69/653

(57) ABSTRACT

A radiation-sensitive resin composition includes: a resin including a structural unit represented by formula (1); a radiation-sensitive acid generator; and a solvent. R¹ is a hydrogen atom, a fluorine atom, a methyl group, or a trifluoromethyl group; L¹ represents a single bond or —COO-L-; L represents a substituted or unsubstituted alkanediyl group; R² represents a monovalent hydrocarbon group having 1 to 20 carbon atoms; L² represents a single bond or a divalent linking group; and Ar represents a group obtained by removing (n+1) hydrogen atoms from an aromatic ring. R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, and at least one R³ is a halogen atom or a halogenated hydrocarbon group.

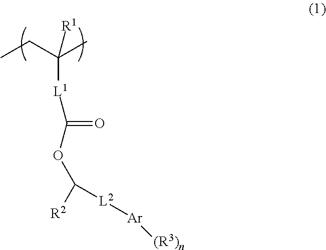

### CROSS-REFERENCE TO RELATED APPLICATIONS

The present application claims priority to Japanese Patent Application No. 2022-085317 filed May 25, 2022, the contents of which are incorporated herein by reference in their entirety.

### BACKGROUND OF THE DISCLOSURE

### Technical Field

The present disclosure relates to a radiation-sensitive resin composition, a method of forming a pattern, a polymer and a compound.

### Description of the Related Art

A photolithography technology using a resist composition has been used for the formation of a fine circuit in a semiconductor device. As a representative procedure, for example, a resist pattern is formed on a substrate by generating an acid by irradiating a coating film of the resist composition with radiation through a mask pattern, and then reacting in the presence of the acid as a catalyst to generate a difference in the solubility of a resin into an alkaline or organic developer between an exposed area and an unexposed area.

In the photolithography technology, the micronization of the pattern is promoted by using short-wavelength radiation such as an ArF excimer laser or by using an immersion exposure method (liquid immersion lithography) in which exposure is performed in a state in which a space between a lens of an exposure apparatus and a resist film is filled with a liquid medium.

While efforts for further technological development are in progress, a technique has been proposed in which a quencher (acid diffusion controlling agent) is blended in a resist composition, and an acid diffused to an unexposed area is captured by a salt exchange reaction to improve lithographic performance with ArF exposure (see, JP-B-5556765). In addition, as a next-generation technology, lithography using shorter-wavelength radiation such as an electron beam, an X-ray, and extreme ultraviolet (EUV) has also been explored.

### SUMMARY OF THE DISCLOSURE

According to an aspect of the present disclosure, a radiation-sensitive resin composition includes: a resin including a structural unit represented by formula (1); a radiation-sensitive acid generator; and a solvent.

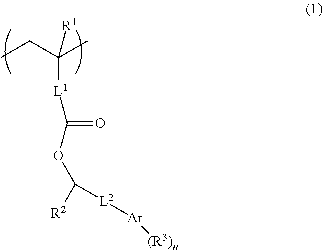

R¹ is a hydrogen atom, a fluorine atom, a methyl group, or a trifluoromethyl group; L¹ represents a single bond or —COO-L-; L represents a substituted or unsubstituted alkanediyl group; R² represents a monovalent hydrocarbon group having 1 to 20 carbon atoms; L² represents a single bond or a divalent linking group; Ar represents a group obtained by removing (n+1) hydrogen atoms from an aromatic ring; and n is an integer of 1 or more. When n is 1, R³ is a halogen atom or a halogenated hydrocarbon group; and when n is 2 or more, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.

According to another aspect of the present disclosure, a method of forming a pattern includes: directly or indirectly applying the radiation-sensitive resin composition according to claim 1 to a substrate to form a resist film; exposing the resist film to light; and developing the exposed resist film.

According to a further aspect of the present disclosure, a polymer includes a structural unit represented by formula (2) or formula (3).

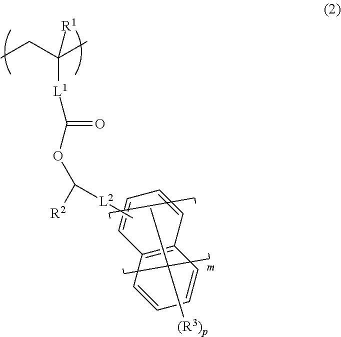

R¹ is a hydrogen atom, a fluorine atom, a methyl group, or a trifluoromethyl group; L¹ represents a single bond or —COO-L-; L represents a substituted or unsubstituted alkanediyl group; R² represents a monovalent hydrocarbon group having 1 to 20 carbon atoms; L² represents a single bond or a divalent linking group; m is 0 or 1; p is an integer of 1 to (5+2m); when p is 1, R³ is a halogen atom or a halogenated hydrocarbon group, and when p is an integer of 2 to (2+2m), each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.

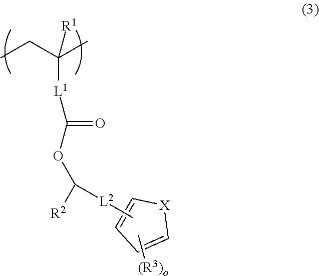

R¹ is a hydrogen atom, a fluorine atom, a methyl group, or a trifluoromethyl group; L¹ represents a single bond or —COO-L-; L represents a substituted or unsubstituted alkanediyl group; R² represents a monovalent hydrocarbon group having 1 to 20 carbon atoms; L² represents a single bond or a divalent linking group; X represents an oxygen atom, —NH—, or a sulfur atom; o is an integer of 1 to 3; when o is 1, R³ is a halogen atom or a halogenated hydrocarbon group; and when o is 2 or 3, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group, a plurality of R³'s are same or different from each other, and at least one R³ is a halogen atom or a halogenated hydrocarbon group.

According to a further aspect of the present disclosure, a compound is represented by formula (5) or formula (6).

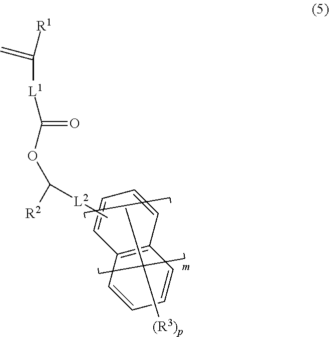

R¹ is a hydrogen atom, a fluorine atom, a methyl group, or a trifluoromethyl group; L¹ represents a single bond or —COO-L-; L represents a substituted or unsubstituted alkanediyl group; R² represents a monovalent hydrocarbon group having 1 to 20 carbon atoms; L² represents a single bond or a divalent linking group; m is 0 or 1; p is an integer of 1 to (5+2m); when p is 1, R³ is a halogen atom, a halogenated hydrocarbon group; and when p is an integer of 2 to (5+2m), each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.

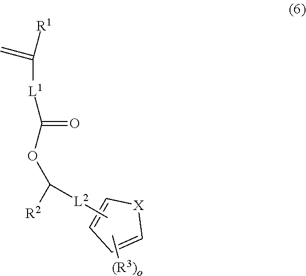

R¹ is a hydrogen atom, a fluorine atom, a methyl group, or a trifluoromethyl group; L¹ represents a single bond or —COO-L-; L represents a substituted or unsubstituted alkanediyl group; R² represents a monovalent hydrocarbon group having 1 to 20 carbon atoms; L² represents a single bond or a divalent linking group; X represents an oxygen atom, —NH—, or a sulfur atom; o is an integer of 1 to 3; when o is 1, R³ is a halogen atom or a halogenated hydrocarbon group; and when o is 2 or 3, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.

### DETAILED DESCRIPTION OF THE PREFERRED EMBODIMENTS

As used herein, the words “a” and “an” and the like carry the meaning of “one or more.” When an amount, concentration, or other value or parameter is given as a range, and/or its description includes a list of upper and lower values, this is to be understood as specifically disclosing all integers and fractions within the given range, and all ranges formed from any pair of any upper and lower values, regardless of whether subranges are separately disclosed. Where a range of numerical values is recited herein, unless otherwise stated, the range is intended to include the endpoints thereof, as well as all integers and fractions within the range. As an example, a stated range of 1-10 fully describes and includes the independent subrange 3.4-7.2 as does the following list of values: 1, 4, 6, 10.

The present disclosure relates, in one embodiment, to a radiation-sensitive resin composition comprising:
a resin having a structural unit represented by formula (1) below (hereinafter also referred to as “structural unit (a)”),
a radiation-sensitive acid generator, and
a solvent:

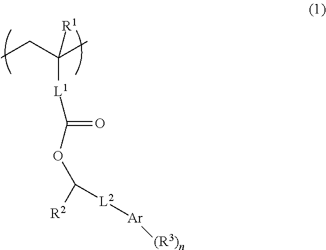

in the formula (1),
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
Ar represents a group obtained by removing (n+1) hydrogen atoms from an aromatic ring;
R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;
n is an integer of 1 or more; and when n is 2 or more, a plurality of R³'s are same or different from each other.

Since the radiation-sensitive resin composition of the present disclosure contains the resin having the structural unit (a), the composition can exhibit superior sensitivity, LWR performance, CD margin performance, and the like at the time of resist pattern formation. The structural unit (a) is presumed to affect the improvement in CD margin performance as well as sensitivity and LWR performance owing to that the structural moiety containing a halogen group such as iodine well absorbs EUV exposure. The scope of the right of the present invention is not necessarily limited by this presumption of the mechanism of action.

In the present disclosure, examples of the “organic group” include a monovalent hydrocarbon group, a group containing a divalent hetero atom-containing group between two adjacent carbon atoms of the monovalent hydrocarbon group, and groups resulting from the hydrocarbon group and the group containing a divalent hetero atom-containing group by substituting some or all of the hydrogen atoms contained therein with a monovalent hetero atom-containing group.

In the present disclosure, the “hydrocarbon group” includes a chain hydrocarbon group, an alicyclic hydrocarbon group, and an aromatic hydrocarbon group unless any particular limitation is imposed on this element. The “hydrocarbon group” includes both a saturated hydrocarbon group and an unsaturated hydrocarbon group. The “chain hydrocarbon group” refers to a hydrocarbon group that does not include any cyclic structure and is composed only of a chain structure, and includes both a linear hydrocarbon group and a branched hydrocarbon group. The “alicyclic hydrocarbon group” refers to a hydrocarbon group that includes only an alicyclic structure as a ring structure and does not include any aromatic ring structure and includes both a monocyclic alicyclic hydrocarbon group and a polycyclic alicyclic hydrocarbon group. However, it is not necessary for the alicyclic hydrocarbon group to be composed only of an alicyclic structure, and the alicyclic hydrocarbon group may include a chain structure as a part thereof. The “aromatic hydrocarbon group” refers to a hydrocarbon group that includes an aromatic ring structure as a ring structure. However, it is not necessary for the aromatic hydrocarbon group to be composed only of an aromatic ring structure, and the aromatic hydrocarbon group may include a chain structure or an alicyclic structure as a part thereof.

The present disclosure relates, in another embodiment, to a method for forming a resist pattern, the method comprising the steps of:
directly or indirectly applying the radiation-sensitive resin composition to a substrate to form a resist film;
exposing the resist film to light; and
developing the exposed resist film.

Since the method for forming a resist pattern of the present disclosure includes the step using the above-described radiation-sensitive resin composition, the method can be utilized, for example, for good pattern formation superior in sensitivity, LWR performance, CD margin performance, and the like.

In addition, the present disclosure relates, in another embodiment, to a polymer comprising a structural unit represented by formula (2) or formula (3) below (hereinafter also referred to as “structural unit (β)” and “structural unit (γ)”, respectively).

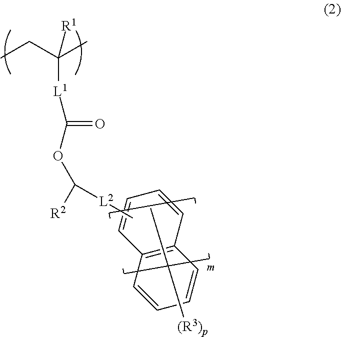

In the formula (2),
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
m is 0 or 1;
R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;
p is an integer of 1 to (5+2m); and when p is 2 or more, a plurality of R³'s are same or different from each other.

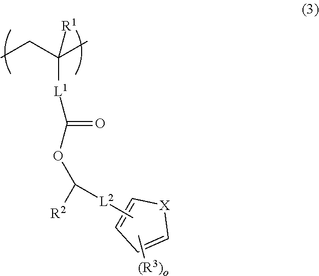

In the formula (3),
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
X represents an oxygen atom, —NH—, or a sulfur atom;
R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;
o is an integer of 1 to 3; and when o is 2 or more, a plurality of R³'s are same or different from each other.

Since the polymer of the present disclosure has the structural unit represented by the formula (2) or the formula (3), the above-described radiation-sensitive resin composition can be produced using this polymer.

In addition, the present disclosure relates, in another embodiment, to a compound represented by formula (5) below or a compound represented by formula (6) below (these are hereinafter also referred to as “compound (5)” and “compound (6)”, respectively).

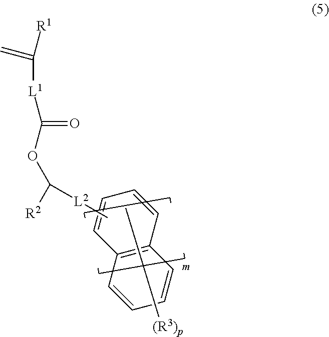

In the formula (5),
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
m is 0 or 1;
R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;
p is an integer of 1 to (5+2m); and when p is 2 or more, a plurality of R³'s are same or different from each other.

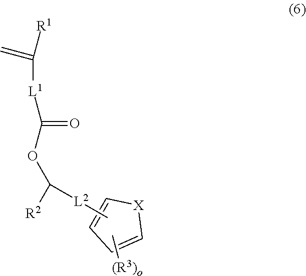

In the formula (6),
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
X represents an oxygen atom, —NH—, or a sulfur atom;
R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;
o is an integer of 1 to 3; and when o is 2 or more, a plurality of R³'s are same or different from each other.

Since the compound (5) and the compound (6) of the present disclosure have the above-mentioned chemical structures, the above-described polymers can be produced using these compounds as monomer components.

Hereinbelow, embodiments of the present invention will specifically be described, but the present invention is not limited to these embodiments.

### <Radiation-Sensitive Resin Composition>

The radiation-sensitive resin composition according to this embodiment comprises a resin having a structural unit represented by the formula (1) shown above (hereinafter also referred to as “resin having a structural unit (α)”), a radiation-sensitive acid generator, and a solvent. The composition may further contain other optional components as long as the effects of the present invention are not impaired. Since the radiation-sensitive resin composition contains the resin having the structural unit (α), the composition can exhibit superior sensitivity, LWR performance, CD margin performance, and the like at the time of resist pattern formation.

### (Resin Having Structural Unit (α))

The resin having a structural unit (α) is an assembly of polymers having a structural unit containing an acid-dissociable group (this structural unit is hereinafter also referred to as “structural unit (I)”) (this resin is hereinafter also referred to as “base resin”). The structural unit (α) is also an acid-dissociable group. While the resin contains the structural unit (α) as an acid-dissociable group, the resin may further contain another acid-dissociable group. The “acid-dissociable group” refers to a group that substitutes for a hydrogen atom of a carboxy group, a phenolic hydroxy group, an alcoholic hydroxy group, a sulfo group, or the like and is dissociated by the action of an acid. The radiation-sensitive resin composition of the present disclosure is superior in patternability because the resin has the structural unit (I) containing the structural unit (α).

In addition to the structural unit (I), the base resin may have a structural unit (II) containing at least one selected from the group consisting of a lactone structure, a cyclic carbonate structure, and a sultone structure, which are described later, or other structural units. Hereinbelow, each of the structural units will be described.

### [Structural Unit (I)]

The structural unit (I) is a structural unit containing an acid-dissociable group, and the resin contains the structural unit (α).

### [Structural Unit (α)]

The structural unit (α) is represented by the following formula (1).

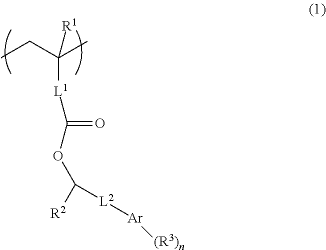

In the above formula (1), R¹ is a hydrogen atom, a fluorine atom, a methyl group, or a trifluoromethyl group.

In the above formula (1), L¹ represents a single bond or —COO-L-.

In the above formula (1), L represents a substituted or unsubstituted alkanediyl group.

Examples of the substituted or unsubstituted alkanediyl group represented by the above L in the above formula (1) include a methanediyl group, an ethanediyl group, and a chloromethanediyl group.

In the above formula (1), R² represents a monovalent hydrocarbon group having 1 to 20 carbon atoms.

Examples of the monovalent hydrocarbon group having 1 to 20 carbon atoms represented by the R² in the above formula (1) include chain hydrocarbon groups having 1 to 20 carbon atoms, monovalent alicyclic hydrocarbon groups having 3 to 20 carbon atoms, and monovalent aromatic hydrocarbon groups having 6 to 20 carbon atoms.

Examples of the chain hydrocarbon group having 1 to 20 carbon atoms represented by R² in the above formula (1) include linear or branched saturated hydrocarbon groups having 1 to 20 carbon atoms and linear or branched unsaturated hydrocarbon groups having 2 to 20 carbon atoms. Examples of the linear or branched saturated hydrocarbon group having 1 to 20 carbon atoms include a group obtained by removing one hydrogen atom from an alkane molecule such as a methyl group, an ethyl group, a n-propyl group, an i-propyl group, a n-butyl group, a 2-methylpropyl group, a 1-methylpropyl group, and a t-butyl group. Examples of the linear or branched unsaturated hydrocarbon group having 2 to 20 carbon atoms include a group obtained by removing one hydrogen atom from an alkene molecule such as an ethenyl group, a propenyl group, or a butenyl group; and a group obtained by removing one hydrogen atom from an alkyne molecule such as an ethynyl group, a propynyl group, or a butynyl group.

Examples of the monovalent alicyclic hydrocarbon group having 3 to 20 carbon atoms represented by R² in the above formula (1) include monocyclic or polycyclic saturated hydrocarbon groups and monocyclic or polycyclic unsaturated hydrocarbon groups. Examples of the monocyclic saturated hydrocarbon group include a cyclopentyl group, a cyclohexyl group, a cycloheptyl group, a cyclooctyl group, a cycloundecanyl group, and a cyclododecanyl group. Examples of the polycyclic cycloalkyl group include bridged alicyclic hydrocarbon groups such as a norbornyl group and an adamantyl group. Examples of the monocyclic unsaturated hydrocarbon group include monocyclic cycloalkenyl groups such as a cyclopropenyl group, a cyclobutenyl group, a cyclopentenyl group, a cyclohexenyl group, and a cyclooctenyl group. Examples of the polycyclic unsaturated hydrocarbon group include polycyclic cycloalkenyl groups such as a norbornenyl group. It is to be noted that the bridged alicyclic hydrocarbon group refers to a polycyclic alicyclic hydrocarbon group in which two carbon atoms that constitute an alicyclic ring and are not adjacent to each other are bonded by a divalent linking group containing one or more carbon atoms.

Examples of the monovalent aromatic hydrocarbon group having 6 to 20 carbon atoms represented by R² in the above formula (1) include aryl groups, such as a phenyl group, a tolyl group, a xylyl group, a naphthyl group, an anthryl group, and a pyrenyl group; and aralkyl groups, such as a benzyl group, a phenethyl group, and a naphthylmethyl group.

In the above formula (1), L² represents a single bond or a divalent linking group.

Examples of the divalent linking group represented by L² in the above formula (1) include a divalent linear or branched hydrocarbon group having 1 to 10 carbon atoms, a divalent alicyclic hydrocarbon group having 4 to 12 carbon atoms, and a group composed of one or more among these hydrocarbon groups and at least one group among —CO—, —O—, —NH—, and —S.

In the above formula (1), Ar represents a group obtained by removing (n+1) hydrogen atoms from an aromatic ring.

Examples of the aromatic ring represented by Ar in the above formula (1) include an aromatic hydrocarbon ring having 6 to 20 carbon atoms and an aromatic heterocyclic ring having 6 to 20 carbon atoms.

Examples of the aromatic hydrocarbon ring having 6 to 20 carbon atoms represented by Ar in the above formula (1) include aryl groups such as a benzene ring, a triphenyl ring, a xylene ring, a naphthalene ring, an anthracene ring, and a pyrene ring.

Examples of the aromatic heterocyclic ring having 6 to 20 carbon atoms represented by Ar in the above formula (1) include a pyrrole ring, a pyridine ring, a furan ring, and a thiophene ring.

In the formula (1), R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;

Examples of the halogen atom represented by R³ in the above formula (1) include a fluorine atom, a chlorine atom, a bromine atom, and an iodine atom.

Examples of the halogenated hydrocarbon group represented by R³ in the above formula (1) include a monovalent hydrocarbon group having 1 to 10 carbon atoms in which some or all of the hydrogen atoms are substituted with a halogen atom. The monovalent hydrocarbon group having 1 to 10 carbon atoms is the same as in the case of the following monovalent hydrocarbon group having 1 to 10 carbon atoms represented by R³.

Examples of the monovalent hydrocarbon group having 1 to 10 carbon atoms represented by the R³ in the above formula (1) include monovalent chain hydrocarbon groups having 1 to 10 carbon atoms, monovalent alicyclic hydrocarbon groups having 3 to 10 carbon atoms, and monovalent aromatic hydrocarbon groups having 6 to 10 carbon atoms.

Examples of the chain hydrocarbon group having 1 to 10 carbon atoms represented by R³ in the above formula (1) include linear or branched monovalent saturated hydrocarbon groups having 1 to 10 carbon atoms and linear or branched monovalent unsaturated hydrocarbon groups having 2 to 10 carbon atoms.

Examples of the monovalent alicyclic hydrocarbon group having 3 to 10 carbon atoms represented by R³ in the above formula (1) include those having 3 to 10 carbon atoms among the monovalent alicyclic hydrocarbon groups recited as examples for R².

Examples of the monovalent aromatic hydrocarbon group having 6 to 10 carbon atoms represented by R³ in the above formula (1) include aryl groups, such as a phenyl group, a tolyl group, a xylyl group, and a naphthyl group; and aralkyl groups, such as a benzyl group, a phenethyl group, and a naphthylmethyl group.

Examples of the monovalent alkyl ether group having 1 to 10 carbon atoms represented by R³ in the above formula (1) include a monovalent chain alkyl ether group having 1 to 10 carbon atoms and a monovalent alicyclic alkyl ether group having 3 to 10 carbon atoms.

Examples of the monovalent chain alkyl ether group having 1 to 10 carbon atoms represented by R³ in the above formula (1) include a monovalent linear or branched alkyl ether group having 1 to 10 carbon atoms and a monovalent linear or branched unsaturated alkyl ether group having 1 to 10 carbon atoms.

Examples of the monovalent alicyclic alkyl ether group having 3 to 10 carbon atoms represented by R³ in the above formula (1) include a monocyclic or polycyclic saturated alkyl ether group and a monocyclic or polycyclic unsaturated alkyl ether group.

In the formula (1), n is an integer of 1 or more. When n is 2 or more, the plurality of R³'s are the same or different from each other. The upper limit number of n is determined according to the type of the ring of Ar, and for example, in the case of an N-membered ring, “N-1” excluding the bonding site to L² is the upper limit number of n. More specifically, when Ar is a 6-membered benzene ring, the upper limit of n is 5. Preferably, n is an integer of 1 to 3.

Preferably, the structural unit represented by the formula (1) is, for example, a structural unit represented by the following formula (2) or a structural unit represented by the following formula (3) (these are referred to as “structural unit (B)” and “structural unit (γ)”, respectively).

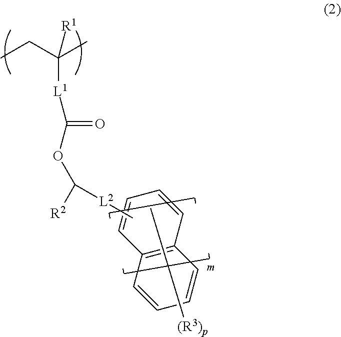

In the formula (2), R¹, L¹, L, R², L², and R³ are the same as those in the formula (1).

In the formula (2), m is 0 or 1.

In the formula (2), p is an integer of 1 to (5+2m). When p is 2 or more, the plurality of R³'s are the same or different from each other.

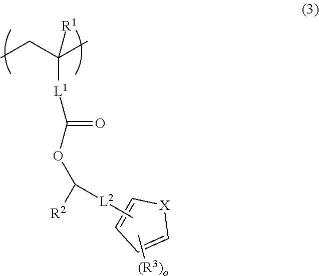

In the formula (3), R¹, L¹, L, R², L², and R³ are the same as those in the formula (1).

In the formula (3), X represents an oxygen atom, —NH—, or a sulfur atom.

In the formula (1), o is an integer of 1 to 3. When o is 2 or more, the plurality of R³'s are the same or different from each other.

Examples of the monomer component for obtaining the structural unit (α) represented by the formula (1) (or the structural unit (β) represented by the formula (2) or the structural unit (γ) represented by the formula (3)) include compounds represented by the following formulas (M-1) to (M-32).

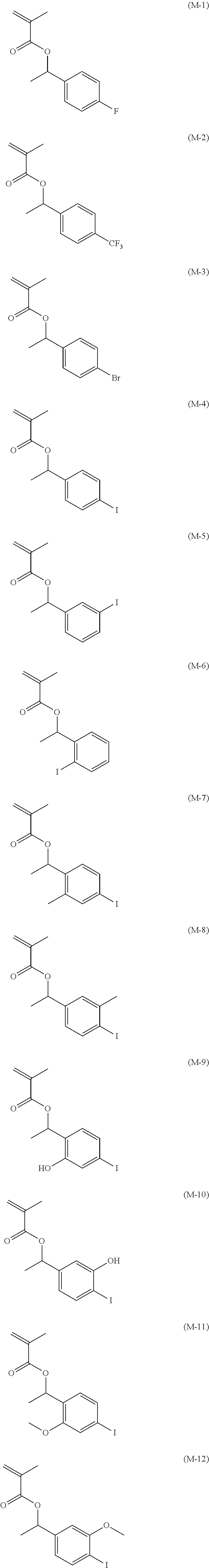

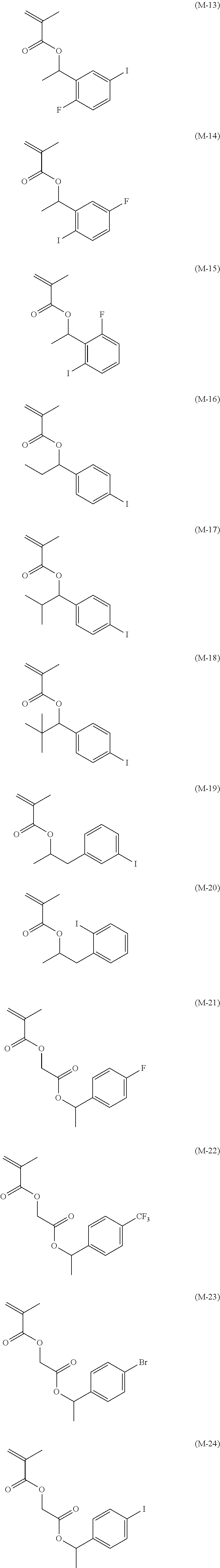

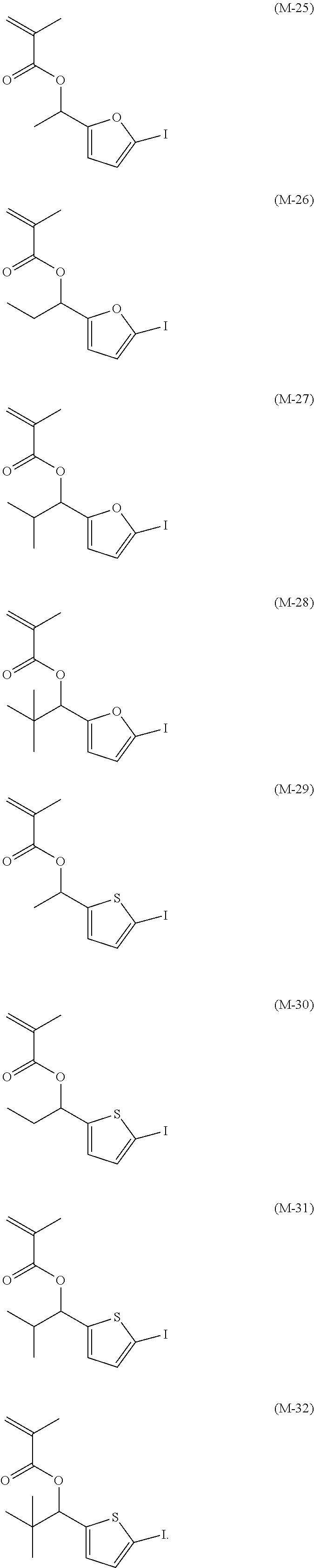

The base resin may contain one type of the structural unit (α) or two or more types of the structural unit (α) in combination.

The content of the structural unit (α) (a total content is taken when a plurality of types are contained) is preferably 1 mol % or more based on all structural units constituting the base resin, and may be, for example, 5 mol % or more, 10 mol % or more, 15 mol % or more, 20 mol % or more, 25 mol % or more, 30 mol % or more, 35 mol % or more, or the like. The content is preferably 80 mol % or less, and may be 75 mol % or less, 70 mol % or less, 65 mol % or less, or the like. When the content of the structural unit (α) is adjusted to within the above range, the patternability of the radiation-sensitive resin composition of the present disclosure can be further improved.

[Structural Unit (I) Other than Structural Unit (α)]

The structural unit (I) may contain other acid-dissociable groups than the structural unit (α). The structural unit (I) is not particularly limited as long as it contains an acid-dissociable group, and examples thereof include a structural unit having a tertiary alkyl ester moiety, a structural unit having a structure in which a hydrogen atom of a phenolic hydroxy group is substituted with a tertiary alkyl group, and a structural unit having an acetal bond. From the viewpoint of improving patternability, a structural unit represented by the following formula (4) (hereinafter also referred to as “structural unit (I-1)”) is preferable.

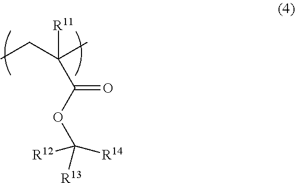

In the formula (4),
R¹¹ is a hydrogen atom, a fluorine atom, a methyl group, or a trifluoromethyl group;
R¹² is a monovalent hydrocarbon group having 1 to 20 carbon atoms; and
R¹³ and R¹⁴ each independently are a monovalent chain hydrocarbon group having 1 to 10 carbon atoms or a monovalent alicyclic hydrocarbon group having 3 to 20 carbon atoms, or represent a divalent alicyclic hydrocarbon group having 3 to 20 carbon atoms composed of those groups and the carbon atoms to which those groups are bonded wherein those groups are combined with each other.

In the formula (4), from the viewpoint of the copolymerizability of a monomer that affords the structural unit (I), as the R¹¹, a hydrogen atom and a methyl group are preferable, and a methyl group is more preferable.

Examples of the monovalent hydrocarbon group having 1 to 20 carbon atoms represented by the R¹² in the above formula (4) include chain hydrocarbon groups having 1 to 20 carbon atoms, monovalent alicyclic hydrocarbon groups having 3 to 20 carbon atoms, and monovalent aromatic hydrocarbon groups having 6 to 20 carbon atoms.

Examples of the chain hydrocarbon group having 1 to 20 carbon atoms represented by R¹² in the above formula (4) include linear or branched saturated hydrocarbon groups having 1 to 20 carbon atoms and linear or branched unsaturated hydrocarbon groups having 1 to 20 carbon atoms.

Examples of the monovalent alicyclic hydrocarbon group having 3 to 20 carbon atoms represented by R¹² in the above formula (4) include the monovalent alicyclic hydrocarbon groups having 3 to 20 carbon atoms recited as examples for R².

Examples of the monovalent aromatic hydrocarbon group having 6 to 20 carbon atoms represented by R¹² in the above formula (4) include the monovalent aromatic hydrocarbon groups having 6 to 20 carbon atoms recited as examples for R².

Examples of the chain hydrocarbon groups having 1 to 10 carbon atoms represented by R¹³ and R¹⁴ in the above formula (4) include linear or branched saturated hydrocarbon groups having 1 to 10 carbon atoms and linear or branched unsaturated hydrocarbon groups having 1 to 10 carbon atoms.

Examples of the monovalent alicyclic hydrocarbon groups having 3 to 20 carbon atoms represented by the R¹³ and R¹⁴ in the above formula (4) include monovalent monocyclic aliphatic hydrocarbon groups having 3 to 20 carbon atoms, and monovalent bridged alicyclic hydrocarbon groups having 6 to 20 carbon atoms.

Examples of the monovalent monocyclic aliphatic hydrocarbon groups having 3 to 20 carbon atoms represented by R¹³ and R¹⁴ in the formula (4) include a cyclopropyl group, a cyclobutyl group, a cyclopentyl group, a cyclohexyl group, and a cyclooctyl group.

Examples of the monovalent bridged alicyclic hydrocarbon groups having 6 to 20 carbon atoms represented by R¹³ and R¹⁴ in the above formula (4) include a norbornyl group, an adamantyl group, a tricyclodecyl group, and a tetracyclododecyl group.

The divalent alicyclic hydrocarbon group having 3 to 20 carbon atoms composed of the groups represented by R¹³ and the R¹⁴ in the above formula (4) and the carbon atoms to which these groups are bonded wherein these groups are combined with each other is not particularly limited as long as it is a group formed by removing two hydrogen atoms from the carbon atoms contained in the carbon ring of the alicyclic hydrocarbon having the aforementioned number of carbon atoms.

Examples of the structural unit (I) represented by the following formula (4) (“structural unit (I-1)”) include structural units represented by the following formulas (2-1) to (2-7) (hereinafter, also referred to as “structural units (I-2-1) to (I-2-7)”).

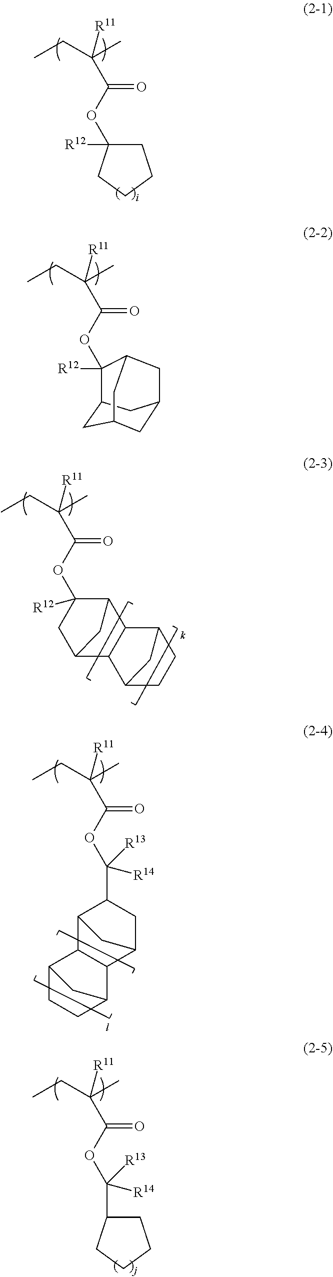

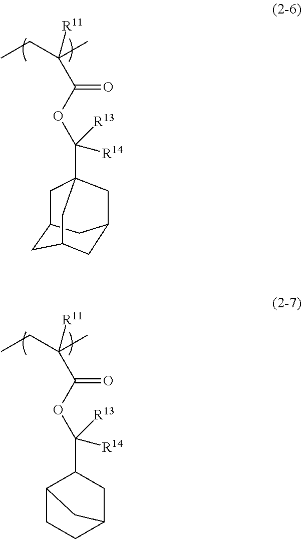

In the formulas (2-1) to (2-7), R¹¹ to R¹⁴ have the same definitions as those in the above formula (4).

In the formula (2-1), i is an integer of 0 to 4. As i, 1 is preferable.

In the formula (2-3), k is 0 or 1.

In the formula (2-4), 1 is 0 or 1.

In the formula (2-5), j is an integer of 0 to 4. As j, 1 is preferable.

In the formulas (2-1) to (2-7), as R¹², a methyl group, an ethyl group, an isopropyl group, a t-butyl group, or a phenyl group is preferable.

In the formulas (2-1) to (2-7), as R¹³ and R¹⁴ each independently, a methyl group, an ethyl group, or an n-propyl group is preferable.

The base resin may contain one type of the structural unit (I) or two or more types of the structural unit (I) in combination.

The content of the structural unit (I) (a total content is taken when a plurality of types are contained) is preferably 1 mol % or more based on all structural units constituting the base resin, and may be, for example, 5 mol % or more, 10 mol % or more, 15 mol % or more, 20 mol % or more, 25 mol % or more, 30 mol % or more, 35 mol % or more, or the like. The content is preferably 80 mol % or less, and may be 75 mol % or less, 70 mol % or less, 65 mol % or less, or the like. When the content of the structural unit (I) is adjusted to within the above range, the patternability of the radiation-sensitive resin composition of the present disclosure can be further improved.

### [Structural Unit (II)]

The structural unit (II) is a structural unit containing at least one structure selected from the group consisting of a lactone structure, a cyclic carbonate structure, and a sultone structure. When the base resin further has the structural unit (II), the solubility of the base resin in a developer can be adjusted, and as a result, the lithographic performance, such as resolution, of the resist film obtained from the radiation-sensitive resin composition of the present disclosure can be improved. In addition, the adhesion between a resist pattern formed from the base resin and a substrate can be improved.

As the structural unit (II), a structural unit containing a lactone structure is preferable, and a structural unit containing a norbornane lactone structure is more preferable.

The content of the structural unit (II) is preferably 20 mol % or more, more preferably 25 mol % or more, and still more preferably 30 mol % or more based on all structural units constituting the base resin. The content by percent is preferably 80 mol % or less, more preferably 75 mol % or less, and still more preferably 70 mol % or less. When the content of the structural unit (II) is adjusted to within the range, the lithographic performance, such as resolution, of a resist film obtained from the radiation-sensitive resin composition of the present disclosure and the adhesion between a resist patter formed and a substrate can be further improved.

### [Structural Unit (III)]

The base resin optionally has other structural units in addition to the structural units (I) and (II). Examples of the other structural unit include a structural unit (III) containing a polar group (excluding those corresponding to the structural unit (I) and the structural unit (II)). When the base resin further has the structural unit (III), the solubility of the base resin in a developer can be adjusted, and as a result, the lithographic performance, such as resolution, of the resist film obtained from the radiation-sensitive resin composition of the present disclosure can be improved. Examples of the polar group include a hydroxy group, a carboxy group, a cyano group, a nitro group, and a sulfonamide group. Among them, a hydroxy group and a carboxy group are preferable, and an alcoholic hydroxy group is more preferable.

When the base resin has the structural unit (III) having a polar group, the content of the structural unit (III) is preferably 5 mol % or more, more preferably 8 mol % or more, and still more preferably 10 mol % or more based on all structural units constituting the base resin. The content by percent is preferably 40 mol % or less, more preferably 35 mol % or less, and still more preferably 30 mol % or less.

### [Structural Unit (IV)]

The base resin optionally has, as other structural units, a structural unit having a phenolic hydroxy group or a structural unit that affords a phenolic hydroxy group by the action of an acid (hereinafter also referred to as “structural unit (IV)”). In particular, the structural unit (IV) can be suitably applied to pattern formation using exposure with radiation having a wavelength of 50 nm or less such as electron beam or EUV.

The structural unit (IV) is preferably represented by the following formula (D).

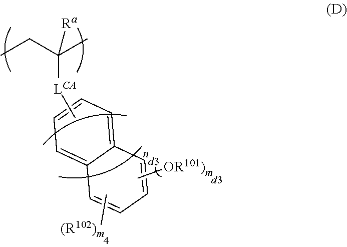

In the formula (D),
R^{α} is a hydrogen atom, a fluorine atom, a methyl group, or a trifluoromethyl group;
L^{CA} is a single bond, —COO—*, or —O—; * is a bond on the aromatic ring side;
R¹⁰¹ is a hydrogen atom or a protective group that is deprotected by the action of an acid; when there are a plurality of R¹⁰¹'s, the plurality of R¹⁰¹'s are the same or different from each other;
R¹⁰² is a cyano group, a nitro group, an alkyl group, a fluorinated alkyl group, an alkoxycarbonyloxy group, an acyl group, or an acyloxy group;
n_{d3} is an integer of 0 to 2, m_{3d} is an integer of 1 to 8, and m_{d4} is an integer of 0 to 8, provided that 1≤m_{d3}+m_{d4}≤2n_{d3}+5 is satisfied.

The R^{α} is preferably a hydrogen atom or a methyl group from the viewpoint of the copolymerizability of a monomer that affords the structural unit (IV).

L^{CA} is preferably a single bond or —COO—*.

Examples of the protective group that is deprotected due to the action of the acid represented by R¹⁰¹ include groups represented by the following formulas (AL-1) to (AL-3).

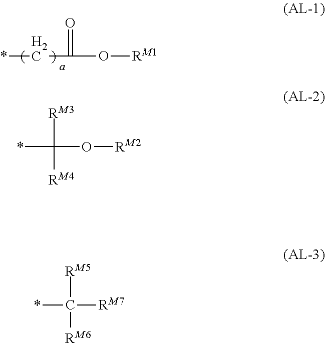

In the formulas (AL-1) and (AL-2), R^{M1} and R^{M2} are monovalent hydrocarbon groups, and may contain a hetero atom such as an oxygen atom, a sulfur atom, a nitrogen atom, or a fluorine atom. The monovalent hydrocarbon group may be linear, branched, or cyclic, and is preferably an alkyl group having 1 to 40 carbon atoms, and more preferably an alkyl group having 1 to 20 carbon atoms. In the formula (AL-1), a is an integer of 0 to 10, and preferably an integer of 1 to 5. In the formulas (AL-1) to (AL-3), * is a bond to another moiety.

In the formula (AL-2), R^{M3} and R^{M4} are each independently a hydrogen atom or a monovalent hydrocarbon group, and may contain a hetero atom such as an oxygen atom, a sulfur atom, a nitrogen atom, or a fluorine atom. The monovalent hydrocarbon group may be linear, branched, or cyclic, and is preferably an alkyl group having 1 to 20 carbon atoms. Any two of R^{M2}, R^{M3}, and R^{M4} may be bonded to each other to form a ring having 3 to 20 carbon atoms together with the carbon atom or the carbon atom and the oxygen atom to which they are bonded. The ring is preferably a ring having 4 to 16 carbon atoms, and particularly preferably an alicyclic ring.

In the formula (AL-3), R^{M5}, R^{M6}, and R^{M7} are each independently a monovalent hydrocarbon group, and may contain a hetero atom such as an oxygen atom, a sulfur atom, a nitrogen atom, or a fluorine atom. The monovalent hydrocarbon group may be linear, branched, or cyclic, and is preferably an alkyl group having 1 to 20 carbon atoms. Any two of R^{M5}, R^{M6}, and R^{M7} may be bonded to each other to form a ring having 3 to 20 carbon atoms together with the carbon atom to which they are bonded. The ring is preferably a ring having 5 to 16 carbon atoms, and particularly preferably an alicyclic ring.

Among them, the protective group that is deprotected due to the action of an acid is preferably a group represented by the formula (AL-3).

Examples of the alkyl group in R¹⁰² include linear or branched alkyl groups having 1 to 8 carbon atoms such as a methyl group, an ethyl group, and a propyl group. Examples of the fluorinated alkyl group include linear or branched fluorinated alkyl groups having 1 to 8 carbon atoms such as a trifluoromethyl group and a pentafluoroethyl group. Examples of the alkoxycarbonyloxy group include chain or alicyclic alkoxycarbonyloxy groups having 2 to 16 carbon atoms such as a methoxycarbonyloxy group, a butoxycarbonyloxy group, and an adamantylmethyloxycarbonyloxy group. Examples of the acyl group include aliphatic or aromatic acyl groups having 2 to 12 carbon atoms such as an acetyl group, a propionyl group, a benzoyl group, and an acryloyl group. Examples of the acyloxy group include aliphatic or aromatic acyloxy groups having 2 to 12 carbon atoms such as an acetyloxy group, a propionyloxy group, a benzoyloxy group, and an acryloyloxy group.

The n_{d3} is preferably 0 or 1, and more preferably 0.

The m₃ is preferably an integer of 1 to 3, and more preferably 1 or 2.

The m₄ is preferably an integer of 0 to 3, and more preferably an integer of 0 to 2.

As the structural unit (IV), structural units represented by the following formulas (D-1) to (D-10) (hereinafter also referred to as “structural units (D-1) to (D-10)”) and the like are preferable.

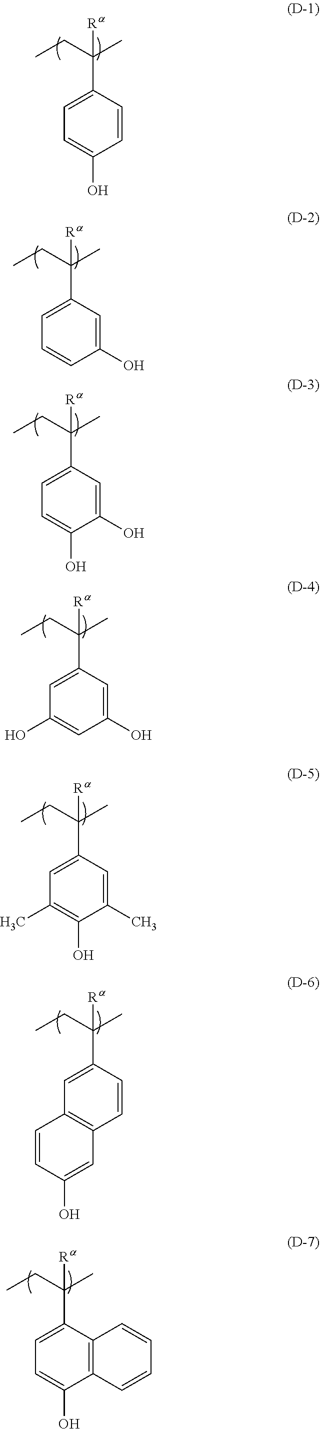

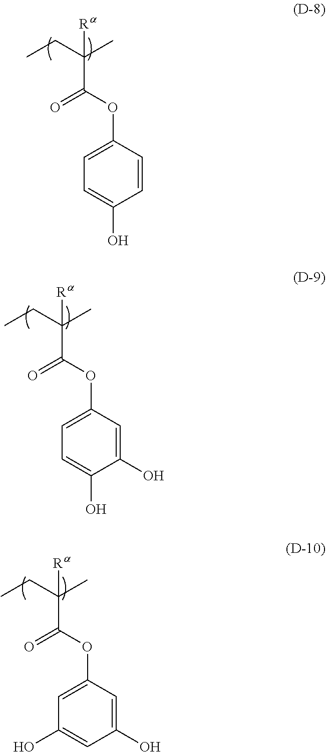

In the formulas (D-1) to (D-10), R^{α} is the same as in the above formula (D).

Among them, the structural units (D-1) to (D-4), (D-6) and (D-8) are preferable.

The content of the structural unit (IV) (when there are a plurality of types of structural unit D, the total content thereof is taken) is preferably 5 mol % or more, more preferably 8 mol % or more, still more preferably 10 mol % or more, and particularly preferably 15 mol % or more based on all structural units constituting the resin. The content is preferably 70 mol % or less, more preferably 60 mol % or less, and still more preferably 50 mol % or less. When the content of the structural unit (IV) is adjusted to within the above range, the sensitivity, CDU performance, and resolution of the radiation-sensitive resin composition can be further improved.

In the case of polymerizing a monomer having a phenolic hydroxy group such as hydroxystyrene, it is preferable to polymerize the monomer with the phenolic hydroxy group protected by a protective group such as an alkali-dissociable group and then deprotect it by hydrolysis to obtain a structural unit (IV).

### (Method for Synthesizing Base Resin)

The base resin can be synthesized by, for example, polymerizing monomers that will afford respective structural units in an appropriate solvent using a radical polymerization initiator or the like.

As the radical polymerization initiator, azobisisobutyronitrile (AIBN) and dimethyl 2,2′-azobisisobutyrate are preferable, and AIBN is more preferable. These radical initiators can be used singly or in combination of two or more types thereof.

The molecular weight of the base resin is not particularly limited, but the weight average molecular weight (Mw) as determined by Gel Permeation Chromatography (GPC) relative to standard polystyrene is preferably 1,000 or more and 50,000 or less, for example, 2,000 or more and 30,000 or less, 3,000 or more and 15,000 or less, 4,000 or more and 12,000 or less, 5,000 or more and 10,000 or less, or 6,000 or more and 9,000 or less. When the Mw of the base resin is less than the lower limit, the heat resistance of the resulting resist film may be deteriorated. When the Mw of the base resin exceeds the above upper limit, the developability of the resist film may be deteriorated.

The ratio (Mw/Mn) of Mw to the number average molecular weight (Mn) of the base resin as determined by GPC relative to standard polystyrene is usually 1 or more and 5 or less, preferably 1 or more and 3 or less, and more preferably 1 or more and 2 or less.

The Mw and the Mn of a resin in the present description are values measured using gel permeation chromatography (GPC) under the following conditions.

GPC column: two G2000HXL, one G3000HXL, one G4000HXL (all manufactured by Tosoh Corporation)
Column temperature: 40° C.
Elution solvent: tetrahydrofuran
Flow rate: 1.0 mL/min
Sample concentration: 1.0% by mass
Amount of sample injected: 100 μL
Detector: differential refractometer
Standard substance: monodisperse polystyrene

The content of the base resin is preferably 70% by mass or more, more preferably 80% by mass or more, and still more preferably 85% by mass based on the total solid content of the radiation-sensitive resin composition.

### (Other Resins)

The radiation-sensitive resin composition of the present embodiment may contain a resin having a higher content by mass of fluorine atoms than the base resin as described above (hereinafter also referred as “high fluorine-containing resin”) as other resin. When the radiation-sensitive resin composition contains the high fluorine-containing resin, the high fluorine-containing resin can be localized in the surface layer of a resist film compared to the base resin, and as a result, the water repellency of the surface of the resist film can be further enhanced in the case of immersion exposure.

The high fluorine-containing resin preferably has, for example, a structural unit represented by the following formula (8) (hereinafter also referred to as “structural unit (V)), and as necessary, may have the structural unit (I) or the structural unit (II) in the base resin.

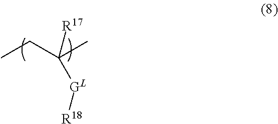

In the above formula (8), R¹⁷ is a hydrogen atom, a methyl group, or a trifluoromethyl group. GL is a single bond, an oxygen atom, a sulfur atom, —COO—, —SO₂ONH—, —CONH—, or —OCONH—. R¹⁸ is a monovalent fluorinated chain hydrocarbon group having 1 to 20 carbon atoms or a monovalent fluorinated alicyclic hydrocarbon group having 3 to 20 carbon atoms.

As the R¹⁷, a hydrogen atom and a methyl group are preferable from the viewpoint of the copolymerizability of a monomer that affords the structural unit (V), and a methyl group is more preferable.

As the GL, a single bond and —COO— are preferable from the viewpoint of the copolymerizability of a monomer that affords the structural unit (V), and —COO— is more preferable.

Examples of the monovalent fluorinated chain hydrocarbon group having 1 to 20 carbon atoms represented by R¹⁸ include groups in which some or all of the hydrogen atoms in the linear or branched chain alkyl group having 1 to 20 carbon atoms are substituted with fluorine atoms.

Examples of the monovalent fluorinated alicyclic hydrocarbon group having 3 to 20 carbon atoms represented by R¹⁸ include monovalent fluorinated alicyclic hydrocarbon groups having 3 to 20 carbon atoms in which some or all of the hydrogen atoms of a mono- or polycyclic hydrocarbon group are substituted with fluorine atoms.

As the R¹⁸, fluorinated chain hydrocarbon groups are preferable, fluorinated alkyl groups are more preferable, and 2,2,2-trifluoroethyl group, 1,1,1,3,3,3-hexafluoropropyl group, and 5,5,5-trifluoro-1,1-diethylpentyl group is even more preferable.

When the high fluorine-containing resin contains the structural unit (V), the content of the structural unit (V) is preferably 30 mol % or more, more preferably 40 mol % or more, still more preferably 45 mol % or more, and particularly preferably 50 mol % or more based on all structural units constituting the high fluorine-containing resin. The content by percent is preferably 95 mol % or less, more preferably 90 mol % or less, and still more preferably 85 mol % or less. When the content of the structural unit (V) is adjusted to within the above range, the content by mass of fluorine atoms in the high fluorine-containing resin can more appropriately be adjusted and the localization in the surface layer of a resist film can be further promoted, and as a result, the water repellency of the resist film can be further enhanced in the case of immersion exposure.

The high fluorine-containing resin may have a fluorine atom-containing structural unit represented by the following formula (f-1) (hereinafter also referred to as structural unit (VI)) in addition to the structural unit (V) or instead of the structural unit (V). When the high fluorine-containing resin has the structural unit (f-1), solubility in an alkaline developer is improved, and the occurrence of development defects can be suppressed.

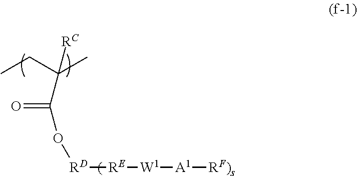

The structural unit (VI) is roughly divided into two cases: a case where it has (x) an alkali-soluble group, and a case where it has (y) a group that is dissociated by the action of an alkali to increase the solubility in an alkaline developer (hereinafter, also simply referred to as “alkali-dissociable group”). Commonly in (x) and (y), in the above formula (f-1), R^{C} is a hydrogen atom, a fluorine atom, a methyl group, or a trifluoromethyl group. R^{D} is a single bond, a hydrocarbon group having 1 to 20 carbon atoms with the valency of (s+1), a structure in which an oxygen atom, a sulfur atom, —NR^{dd}—, a carbonyl group, —COO— or —CONH— is connected to the terminal on R^{E} side of the hydrocarbon group, or a structure in which some of the hydrogen atoms in the hydrocarbon group are substituted with organic groups having a hetero atom. R^{dd} is a hydrogen atom or a monovalent hydrocarbon group having 1 to 10 carbon atoms. s is an integer of 1 to 3.

When the structural unit (VI) has (x) an alkali-soluble group, R^{F} is a hydrogen atom, and A¹ is an oxygen atom, —COO—* or —SO₂O—*. * indicates a site that bonds to R^{F}. W¹ is a single bond, a hydrocarbon group having 1 to 20 carbon atoms, or a divalent fluorinated hydrocarbon group. When A¹ is an oxygen atom, W¹ is a fluorinated hydrocarbon group having a fluorine atom or a fluoroalkyl group on the carbon atom to which A¹ is bonded. R^{E} is a single bond or a divalent organic group having 1 to 20 carbon atoms. When s is 2 or 3, a plurality of R^{E}'s, W¹'s, A¹'s, and R^{F}'s may be the same or different, respectively. When the structural unit (VI) has (x) an alkali-soluble group, affinity to an alkaline developer can be increased, and development defects can be suppressed. As the structural unit (VI) having (x) an alkali-soluble group, a case where A¹ is an oxygen atom and W¹ is a 1,1,1,3, 3,3-hexafluoro-2,2-methanediyl group is particularly preferable.

When the structural unit (VI) has (y) an alkali-dissociable group, R^{F} is a monovalent organic group having 1 to 30 carbon atoms, and A¹ is an oxygen atom, —NR^{aa}, —COO—* or —SO₂O—*. R^{aa} is a hydrogen atom or a monovalent hydrocarbon group having 1 to 10 carbon atoms. * indicates a site that bonds to R^{F}. W¹ is a single bond or a divalent fluorinated hydrocarbon group having 1 to 20 carbon atoms. R^{E} is a single bond or a divalent organic group having 1 to 20 carbon atoms. When A¹ is —COO—* or —SO₂O—*, W¹ or R^{F} has a fluorine atom on a carbon atom bonded to A¹ or on a carbon atom adjacent thereto. When A¹ is an oxygen atom, W¹ and R^{E} are single bonds, R^{D} is a structure in which a carbonyl group is bonded to a terminal on the R^{E} side of a hydrocarbon group having 1 to 20 carbon atoms, and R^{F} is an organic group having a fluorine atom. When s is 2 or 3, a plurality of R^{E}'s, W¹'s, A¹'s, and R^{F}'s may be the same or different, respectively. When the structural unit (VI) has (y) an alkali-dissociable group, the surface of a resist film changes from hydrophobic to hydrophilic in an alkali development step. As a result, the affinity to a developer can be greatly increased, and development defects can be more efficiently suppressed. As the structural unit (VI) having (y) an alkali-dissociable group, a structural unit in which A¹ is —COO—*, and R^{F}, W¹, or both of them have a fluorine atom is particularly preferable.

As the R^{C}, a hydrogen atom and a methyl group are preferable from the viewpoint of the copolymerizability of a monomer that affords the structural unit (VI), and a methyl group is more preferable.

When R^{E} is a divalent organic group, a group having a lactone structure is preferable, a group having a polycyclic lactone structure is more preferable, and a group having a norbornanelactone structure is still more preferable.

When the high fluorine-containing resin contains the structural unit (VI), the content of the structural unit (VI) is preferably 50 mol % or more, more preferably 60 mol % or more, and still more preferably 70 mol % or more based on all structural units constituting the high fluorine-containing resin. The content by percent is preferably 95 mol % or less, more preferably 90 mol % or less, and still more preferably 85 mol % or less. When the content ratio of the structural unit (VI) is adjusted to within the above range, the water repellency of the resist film can be further enhanced in the case of immersion exposure.

The lower limit of the Mw of the high fluorine-containing resin is preferably 1,000, more preferably 2,000, even more preferably 3,000, and particularly preferably 5,000. The upper limit of the Mw is preferably 50,000, more preferably 30,000, even more preferably 20,000, and particularly preferably 15,000.

The lower limit of Mw/Mn of the high fluorine-containing resin is usually 1, and preferably 1.1. The upper limit of the Mw/Mn is usually 5, preferably 3, more preferably 2, and even more preferably 1.9.

The content of the high fluorine-containing resin is preferably 0.1 parts by mass or more, more preferably 0.5 parts by mass or more, still more preferably 1 part by mass or more, and particularly preferably 1.5 parts by mass or more based on 100 parts by mass of the base resin. The content of the high fluorine-containing resin is preferably 15 parts by mass or less, more preferably 12 parts by mass or less, still more preferably 10 parts by mass or less, and particularly preferably 8 parts by mass or less.

When the content of the high fluorine-containing resin is adjusted to within the above range, the high fluorine-containing resin can be more effectively localized in the surface layer of a resist film, and as a result, the water repellency of the surface of the resist film can be further enhanced in the case of immersion exposure. The radiation-sensitive resin composition may contain one type or two or more types of high fluorine-containing resin.

### (Method for Synthesizing High Fluorine-Containing Resin)

The high fluorine-containing resin can be synthesized by the same method as the method for synthesizing a base resin described above.

### (Radiation-Sensitive Acid Generator)

The radiation-sensitive resin composition preferably further comprises a radiation-sensitive acid generator that generates an acid having a pKa lower than that of the acid generated from the acid diffusion controlling agent by irradiation with radiation (exposure to light). When the radiation-sensitive resin composition contains the radiation-sensitive acid generator, the acid generated by exposure to light dissociates the acid-dissociable group (structural unit (I)) of the resin to generate a carboxy group or the like. As a result, the polarity of the resin in the exposed area increases, so that in the case of development with an aqueous alkaline solution, the resin in the exposed area is soluble in the developer, whereas in the case of development with an organic solvent, the resin in the exposed area is hardly soluble in the developer.

The radiation-sensitive acid generator preferably contains an organic acid anion moiety and an onium cation moiety. The organic acid anion moiety preferably has at least one type of anion selected from the group consisting of a sulfonate anion and a sulfonimide anion. Examples of the acid generated by exposure to light include a sulfonic acid and a sulfonimide corresponding to the organic acid anion moiety. The organic acid anion moiety preferably contains an iodine-substituted aromatic ring structure.

In particular, a compound in which one or more fluorine atoms or fluorinated hydrocarbon groups are bonded to a carbon atom adjacent to a sulfonate anion can be suitably employed as a radiation-sensitive acid generator that affords a sulfonic acid by exposure to light.

The radiation-sensitive acid generator is preferably represented by the following formula (A-1) or (A-2).

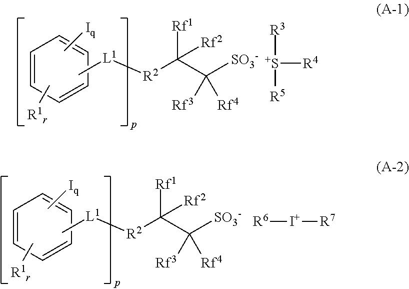

In the formulas (A-1) and (A-2), L¹ is a single bond, an ether linkage, an ester linkage, or an alkylene group having 1 to 6 carbon atoms and optionally containing an ether linkage or an ester linkage. The alkylene group may be linear, branched, or cyclic.

In the formulas (A-1) and (A-2), R¹ is a hydroxy group, a carboxy group, a fluorine atom, a chlorine atom, a bromine atom, or an amino group; or is an alkyl group having 1 to 20 carbon atoms, an alkoxy group having 1 to 20 carbon atoms, an alkoxycarbonyl group having 2 to 10 carbon atoms, an acyloxy group having 2 to 20 carbon atoms, or an alkylsulfonyloxy group having 1 to 20 carbon atoms, each optionally containing a fluorine atom, a chlorine atom, a bromine atom, a hydroxy group, an amino group, or an alkoxy group having 1 to 10 carbon atoms; or is —NR⁸—C(═O)—R⁹ or —NR⁸—C(═O)—O—R⁹, wherein R⁸ is a hydrogen atom or an alkyl group having 1 to 6 carbon atoms and optionally containing a halogen atom, a hydroxy group, an alkoxy group having 1 to 6 carbon atoms, an acyl group having 2 to 6 carbon atoms, or an acyloxy group having 2 to 6 carbon atoms, and R⁹ is an alkyl group having 1 to 16 carbon atoms, an alkenyl group having 2 to 16 carbon atoms, or an aryl group having 6 to 12 carbon atoms and optionally contains a halogen atom, a hydroxy group, an alkoxy group having 1 to 6 carbon atoms, an acyl group having 2 to 6 carbon atoms, or an acyloxy group having 2 to 6 carbon atoms. The alkyl group, alkoxy group, alkoxycarbonyl group, acyloxy group, acyl group, and alkenyl group may be linear, branched, or cyclic.

Among them, R¹ is preferably a hydroxy group, —NR^{B}—C(═O)—R⁹, a fluorine atom, a chlorine atom, a bromine atom, a methyl group, a methoxy group, or the like.

R² is a single bond or a divalent linking group having 1 to 20 carbon atoms when p is 1, and is a trivalent or tetravalent linking group having 1 to 20 carbon atoms when p is 2 or 3, and the linking groups may contain an oxygen atom, a sulfur atom, or a nitrogen atom.

In the formulas (A-1) and (A-2), Rf¹ to Rf⁴ are each independently a hydrogen atom, a fluorine atom, or a trifluoromethyl group, and at least one of Rf¹ to Rf⁴ is a fluorine atom or a trifluoromethyl group. Rf¹ and Rf² may be combined to form a carbonyl group. In particular, both Rf³ and Rf⁴ are preferably fluorine atoms.

In the formulas (A-1) and (A-2), R³, R⁴, R⁵, R⁶, and R⁷ are each independently a monovalent hydrocarbon group having 1 to 20 carbon atoms and optionally containing a heteroatom. When the onium cation moiety of the radiation-sensitive acid generator has a fluorine atom, at least one of R³, R⁴, and R⁵ contains one or more fluorine atoms, and at least one of R⁶ and R⁷ contains one or more fluorine atoms. Any two of R³, R⁴, and R⁵ may be bonded to each other to form a ring together with the sulfur atom to which they are bonded. The monovalent hydrocarbon group may be linear, branched, or cyclic, and examples thereof include an alkyl group having 1 to 12 carbon atoms, an alkenyl group having 2 to 12 carbon atoms, an alkynyl group having 2 to 12 carbon atoms, an aryl group having 6 to 20 carbon atoms, and an aralkyl group having 7 to 12 carbon atoms. Some or all of the hydrogen atoms of these groups may be replaced by a hydroxy group, a carboxy group, a halogen atom, a cyano group, an amide group, a nitro group, a mercapto group, a sultone group, a sulfone group, or a sulfonium salt-containing group, and some of the carbon atoms of these groups may be replaced by an ether linkage, an ester linkage, a carbonyl group, a carbonate group, or a sulfonic acid ester linkage.

In the formulas (A-1) and (A-2), p is an integer satisfying 1≤p≤3. q and r are integers satisfying 0≤q≤5, 0≤r≤3, and 0≤q+r≤5. q is preferably an integer satisfying 1 q 3, and more preferably 2 or 3. r is preferably an integer satisfying 0≤r≤2.

Examples of the organic acid anion moiety of the radiation-sensitive acid generators represented by the formulas (A-1) and (A-2) include, but are not limited to, those shown below. Organic acid anion moieties having no iodine-substituted aromatic ring structure that can be suitably employed include structures in which the iodine atoms in the formulas shown below are replaced by an atom or group other than an iodine atom such as a hydrogen atom or other substituent.

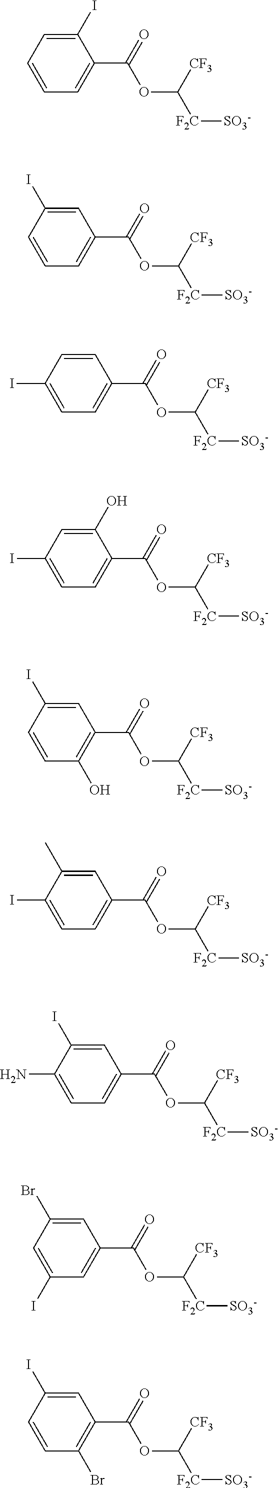

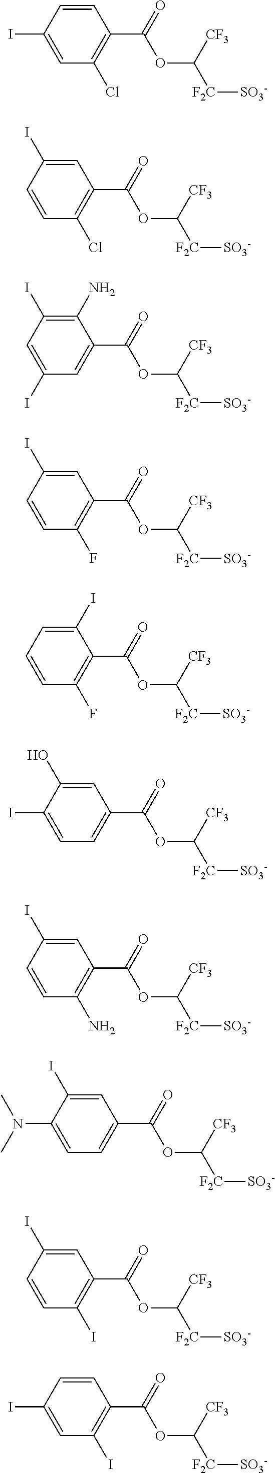

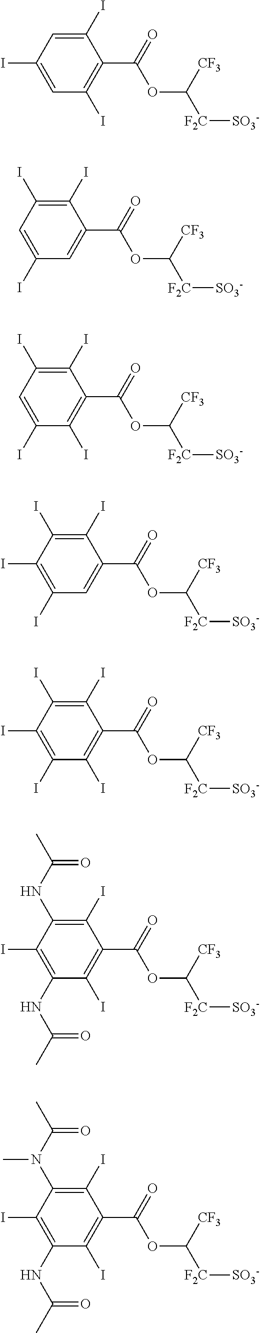

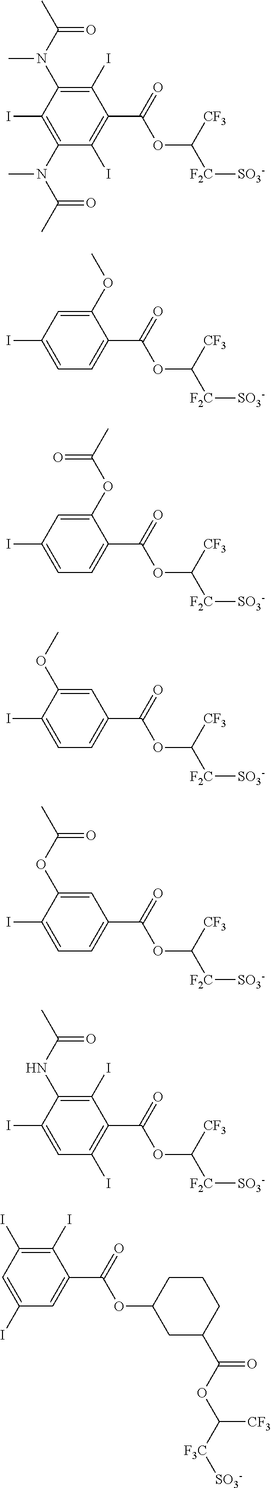

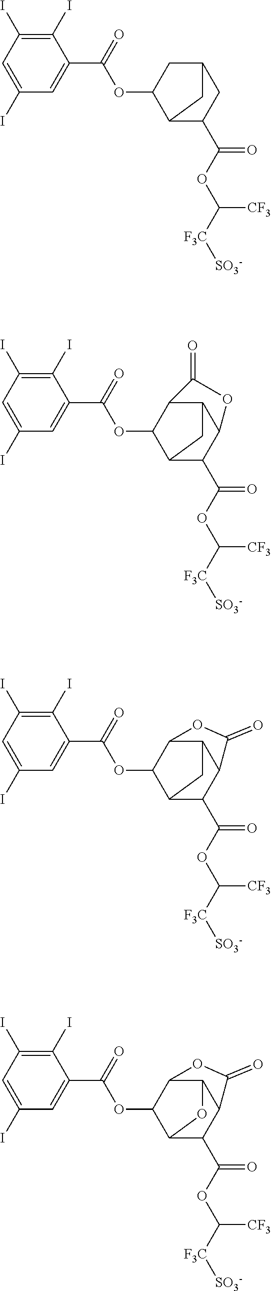

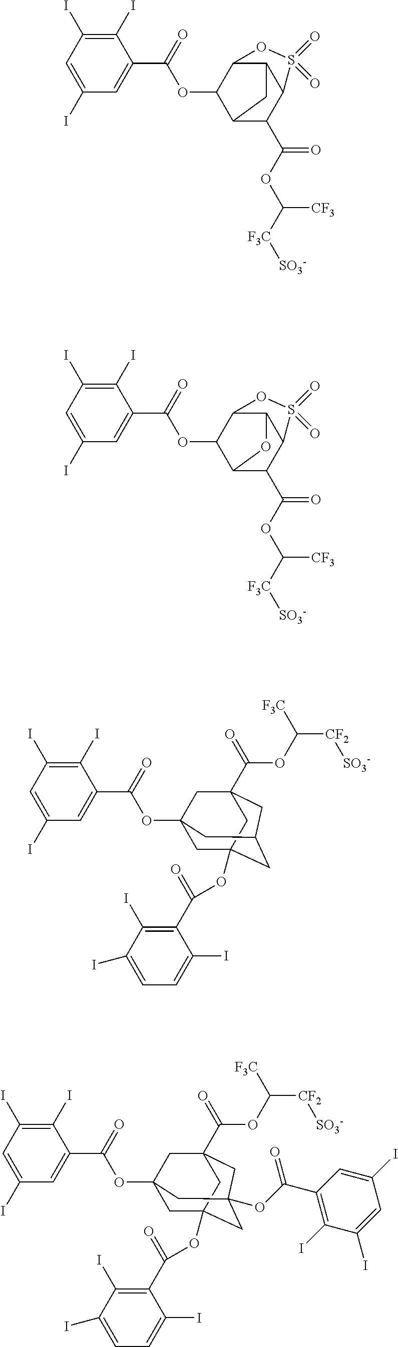

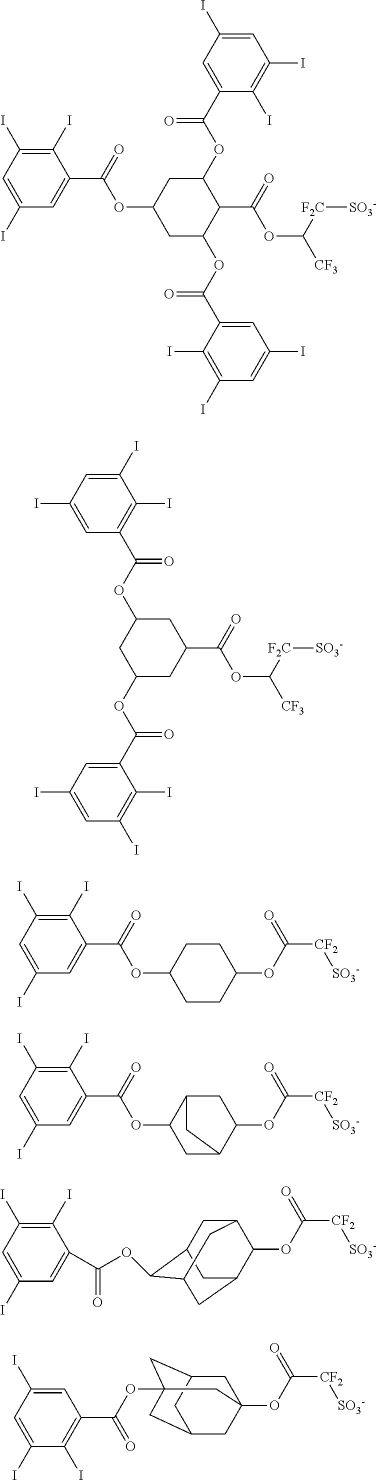

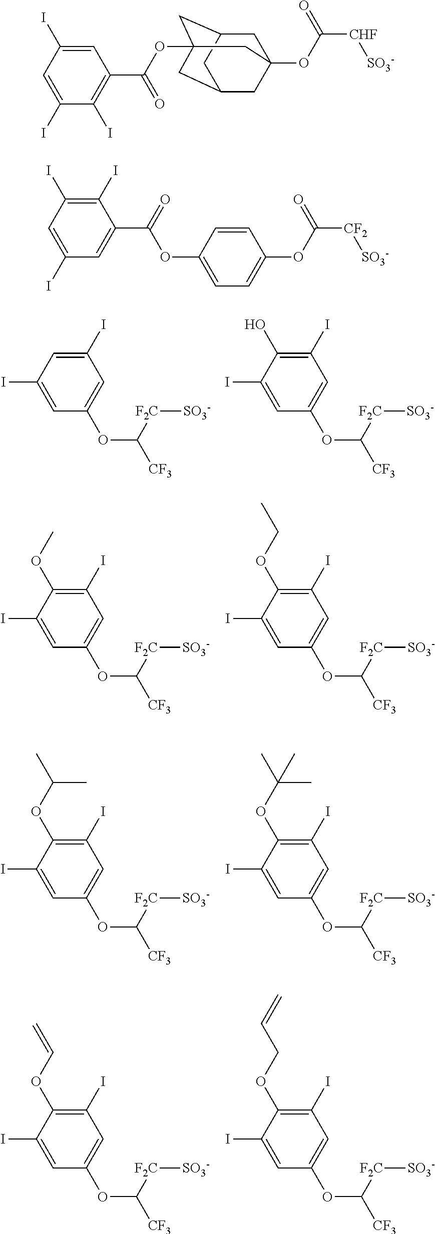

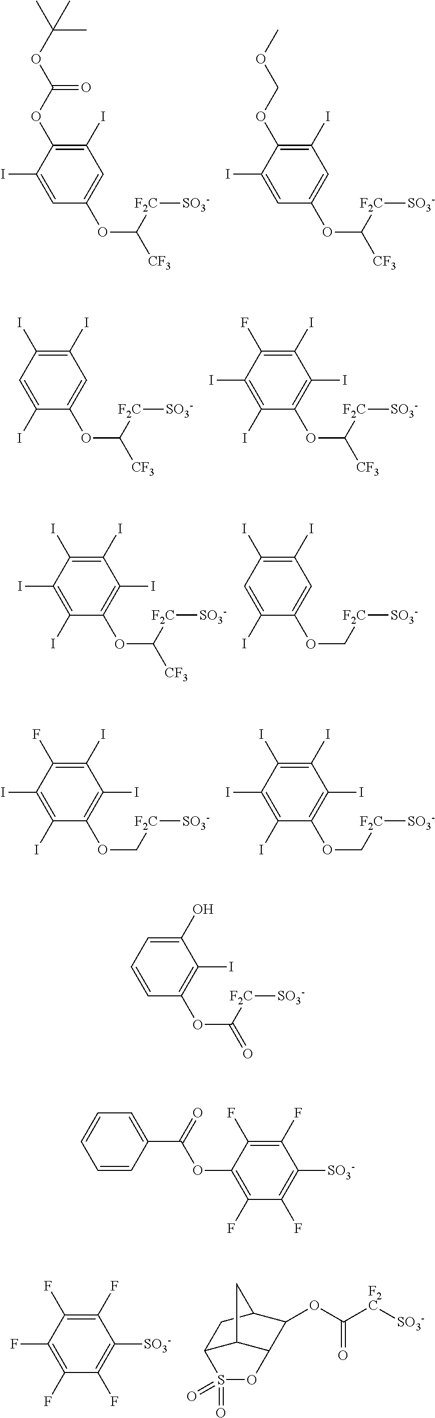

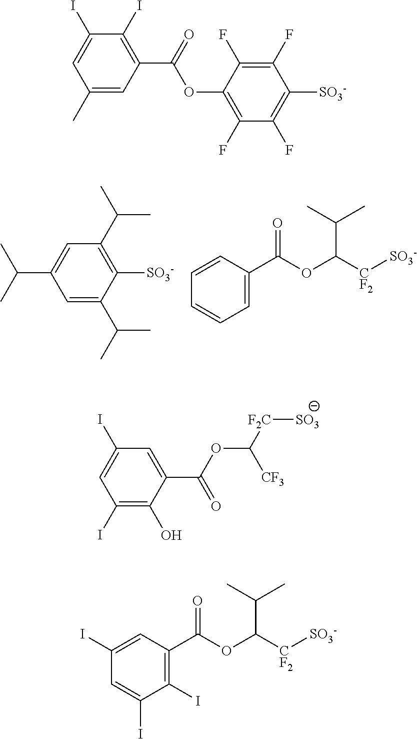

As the onium cation moiety in the radiation-sensitive acid generator represented by the formula (A-1), an onium cation containing an aromatic ring structure having a fluorine atom is preferable, and an onium cation represented by the following formula (Q-1) is more preferable.

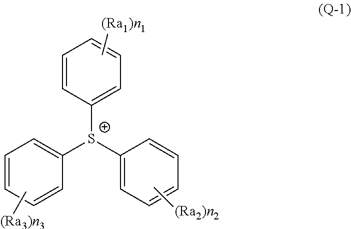

In the formula (Q-1), Ra₁ and Ra₂ each independently represent a substituent. n₁ represents an integer of 0 to 5, and when n₁ is 2 or more, the plurality of Ra₁'s may be the same or different. n₂ represents an integer of 0 to 5, and when n₂ is 2 or more, the plurality of Ra₂'s may be the same or different. n₃ represents an integer of 0 to 5, and when n₃ is 2 or more, the plurality of Ra₃'s may be the same or different. Ra₃ represents a fluorine atom or a group having one or more fluorine atoms. Ra₁ and Ra₂ may be linked to each other to form a ring. When n₁ is 2 or more, the plurality of Ra₁'s may be linked to each other to form a ring. When n₂ is 2 or more, the plurality of Ra₂'s may be linked to each other to form a ring.

The substituent represented by Ra₁ and Ra₂ is preferably an alkyl group, a cycloalkyl group, an alkoxy group, a cycloalkyloxy group, an alkoxycarbonyl group, an alkylsulfonyl group, a hydroxy group, a halogen atom, or a halogenated hydrocarbon group.

The alkyl group as Ra₁ and Ra₂ may be either a linear alkyl group or a branched alkyl group. As the alkyl group, alkyl groups having 1 to 10 carbon atoms are preferable, and examples thereof include those having 1 to 6 carbon atoms among the chain hydrocarbon groups having 1 to 20 carbon atoms recited above as examples for R². Among them, a methyl group, an ethyl group, a n-butyl group, and a t-butyl group are particularly preferable.

Examples of the cycloalkyl group of Ra₁ and Ra₂ include a monocyclic or polycyclic cycloalkyl group (preferably, a cycloalkyl group having 3 to 20 carbon atoms), and the monovalent alicyclic hydrocarbon group having 3 to 20 carbon atoms recited above as examples for R². Among these, a cyclopropyl group, a cyclopentyl group, a cyclohexyl group, a cyclohexyl group, a cycloheptyl group, and a cyclooctyl group are particularly preferable.

Examples of the alkyl group moiety of the alkoxy group as Ra₁ and Ra₂ include those listed above as the alkyl group as Ra₁ and Ra₂. As the alkoxy group, a methoxy group, an ethoxy group, a n-propoxy group, and a n-butoxy group are particularly preferable.

Examples of the cycloalkyl group moiety of the cycloalkyloxy group as Ra₁ and Ra₂ include those listed above as the cycloalkyl group as Ra₁ and Ra₂. As the cycloalkyloxy group, a cyclopentyloxy group and a cyclohexyloxy group are particularly preferable.

Examples of the alkoxy group moiety of the alkoxycarbonyl group as Ra₁ and Ra₂ include those listed above as the alkoxy group as Ra₁ and Ra₂. As the alkoxycarbonyl group, a methoxycarbonyl group, an ethoxycarbonyl group, and a n-butoxycarbonyl group are particularly preferable.

Examples of the alkyl group moiety of the alkylsulfonyl group as Ra₁ and Ra₂ include those listed above as the alkyl group as Ra₁ and Ra₂. Examples of the cycloalkyl group moiety of the cycloalkylsulfonyl group as Ra₁ and Ra₂ include those listed above as the cycloalkyl group as Ra₁ and Ra₂. As the alkylsulfonyl group or the cycloalkylsulfonyl group, a methanesulfonyl group, an ethanesulfonyl group, a n-propanesulfonyl group, a n-butanesulfonyl group, a cyclopentanesulfonyl group, and a cyclohexanesulfonyl group are particularly preferable.

Each of the groups Ra₁ and Ra₂ may further have a substituent. Examples of the substituent include a halogen atom such as a fluorine atom (preferably a fluorine atom), a hydroxy group, a carboxy group, a cyano group, a nitro group, an alkoxy group, a cycloalkyloxy group, an alkoxyalkyl group, a cycloalkyloxyalkyl group, an alkoxycarbonyl group, a cycloalkyloxycarbonyl group, an alkoxycarbonyloxy group, and a cycloalkyloxycarbonyloxy group.

Examples of the halogen atom as Ra₁ and Ra₂ include a fluorine atom, a chlorine atom, a bromine atom, and an iodine atom, and a fluorine atom is preferable.

As the halogenated hydrocarbon group as Ra₁ and Ra₂, a halogenated alkyl group is preferable. Examples of the alkyl group and the halogen atom constituting the halogenated alkyl group include those described above. Among them, a fluorinated alkyl group is preferable, and CF₃ is more preferable.

As described above, Ra₁ and Ra₂ may be linked to each other to form a ring (namely, a heterocyclic ring containing a sulfur atom). When n₁ is 2 or more, a plurality of Ra₁'s may be linked to each other to form a ring, and when n₂ is 2 or more, a plurality of Ra₂'s may be linked to each other to form a ring. Examples thereof include an embodiment in which two Ra₁'s are linked to each other to form a naphthalene ring together with a benzene ring to which they are bonded.

Ra₃ is a fluorine atom or a group having a fluorine atom. Examples of the group having a fluorine atom include groups in which an alkyl group, a cycloalkyl group, an alkoxy group, a cycloalkyloxy group, an alkoxycarbonyl group, and an alkylsulfonyl group as Ra₁ and Ra₂ are substituted with a fluorine atom. Among them, fluorinated alkyl groups are suitable, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉ are more suitable, and CF₃ is particularly suitable.

Ra₃ is preferably a fluorine atom or CF₃, and more preferably a fluorine atom.

n₁ and n₂ are each independently preferably an integer of 0 to 3, and preferably an integer of 0 to 2.

n₃ is preferably an integer of 1 to 3, and more preferably 1 or 2.

Examples of such an onium cation moiety represented by the formula (Q-1) include those shown below. While all of those shown below are sulfonium cation moieties having a fluorine-substituted aromatic ring structure, onium cation moieties having no fluorine-substituted aromatic ring structure that can be suitably employed include structures in which the fluorine atoms or CF₃ in the formulas shown below are replaced by an atom or group other than a fluorine atom such as a hydrogen atom or other substituent.

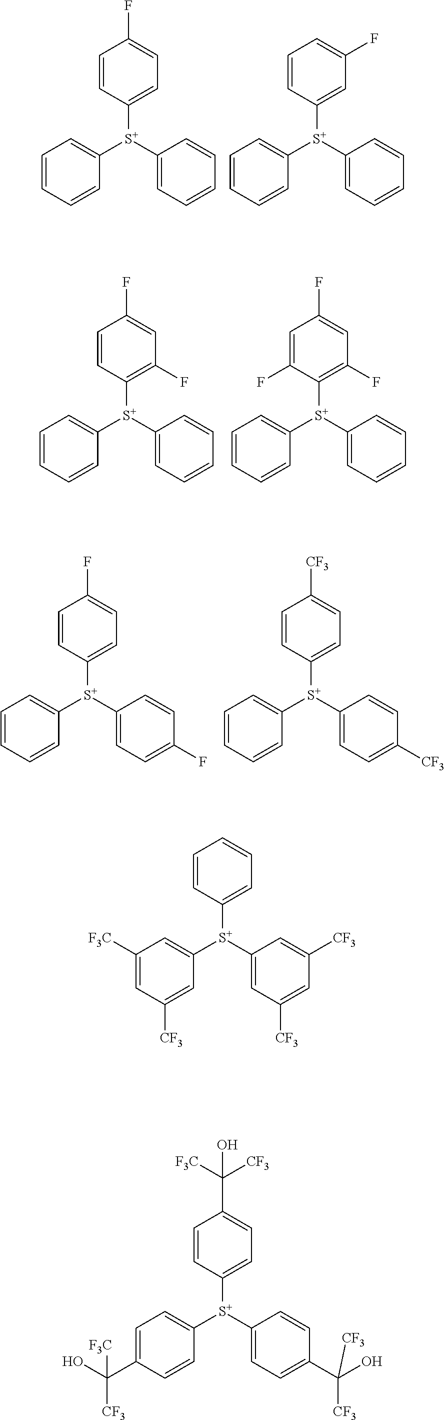

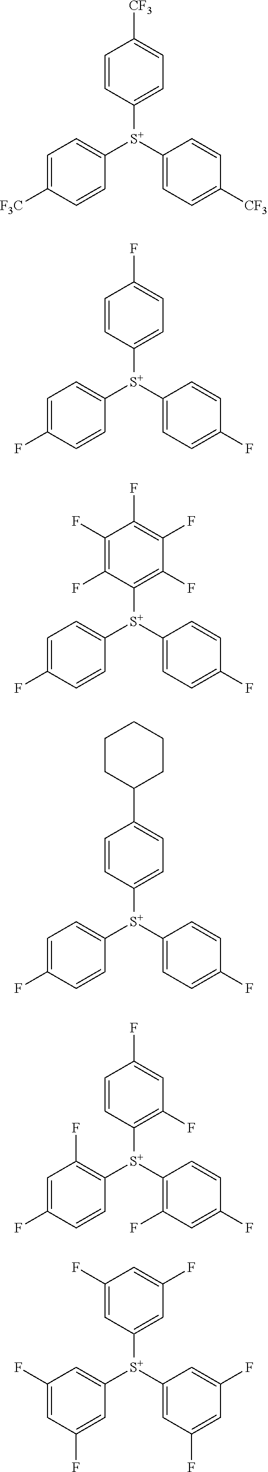 [SMILES: FC(F)(F)c1ccc([S+](c2ccc(C(F)(F)F)cc2)c2ccc(C(F)(F)F)cc2)cc1 ; Fc1ccc([S+](c2ccc(F)cc2)c2ccc(F)cc2)cc1 ; Fc1ccc([S+](c2ccc(F)cc2)c2c(F)c(F)c(F)c(F)c2F)cc1 ; Fc1ccc([S+](c2ccc(F)cc2)c2ccc(C3CCCCC3)cc2)cc1 ; Fc1ccc([S+](c2ccc(F)cc2F)c2ccc(F)cc2F)c(F)c1 ; Fc1cc(F)cc([S+](c2cc(F)cc(F)c2)c2cc(F)cc(F)c2)c1]

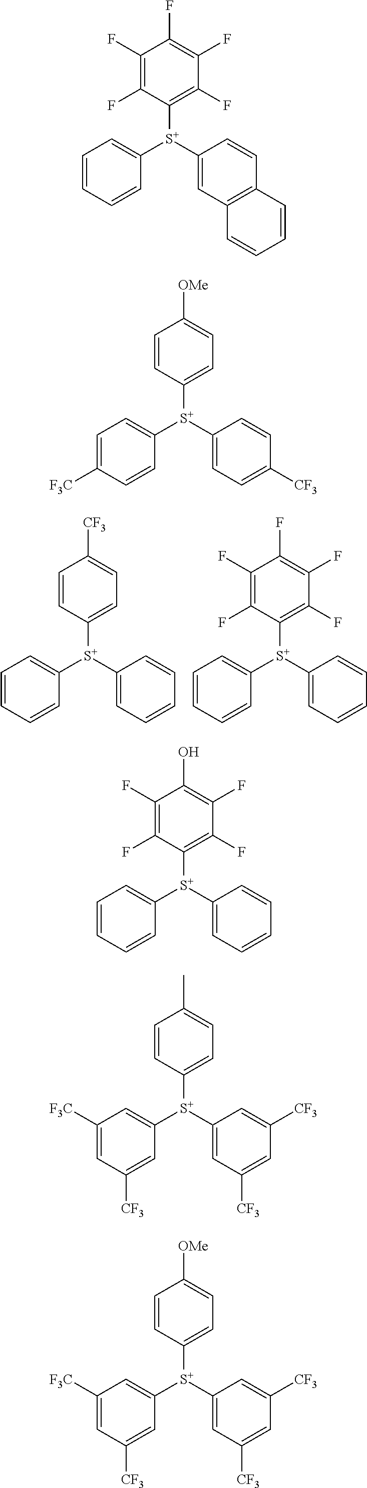

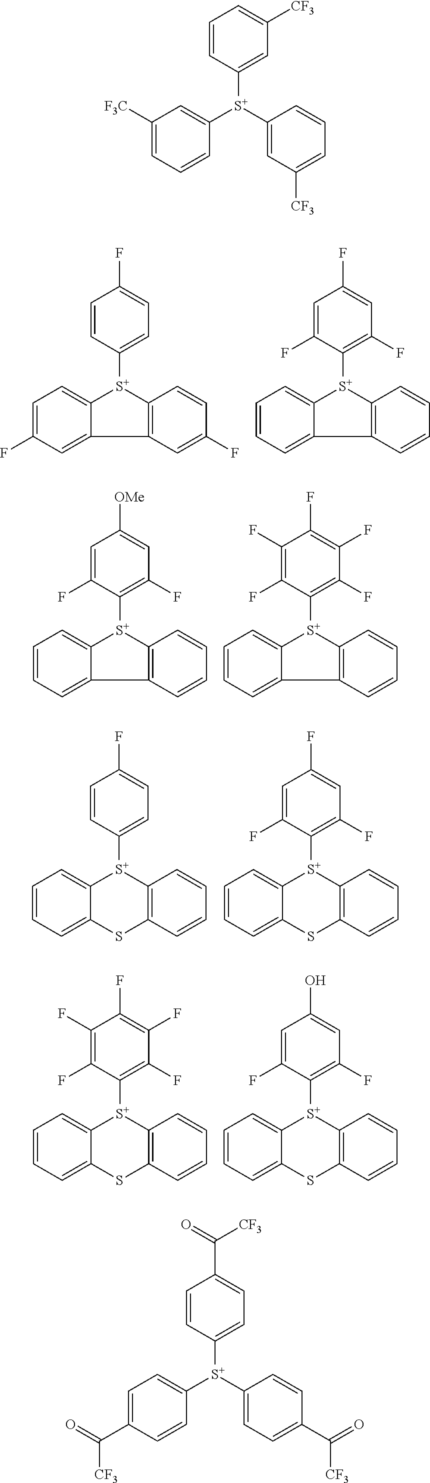

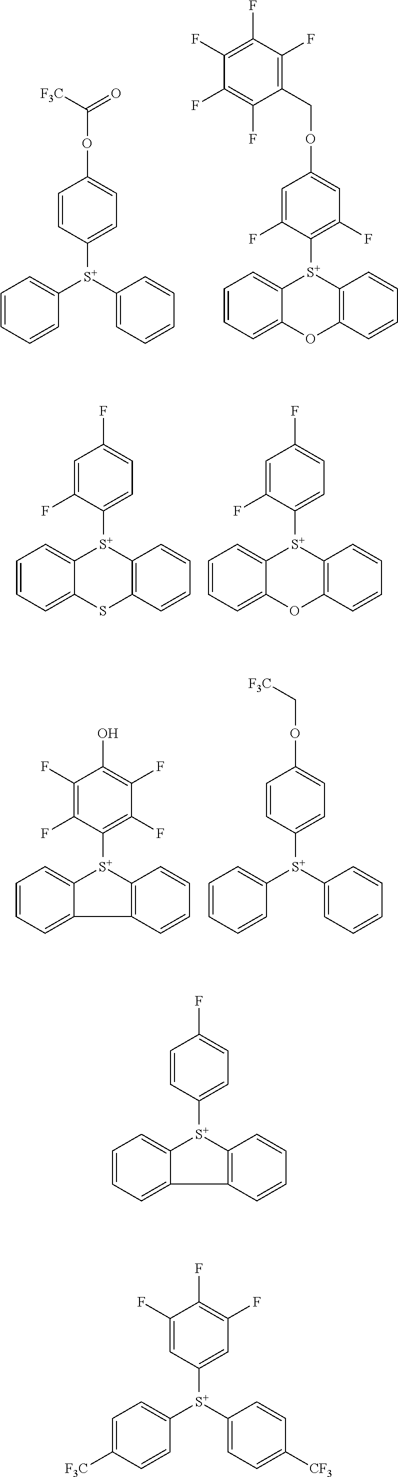

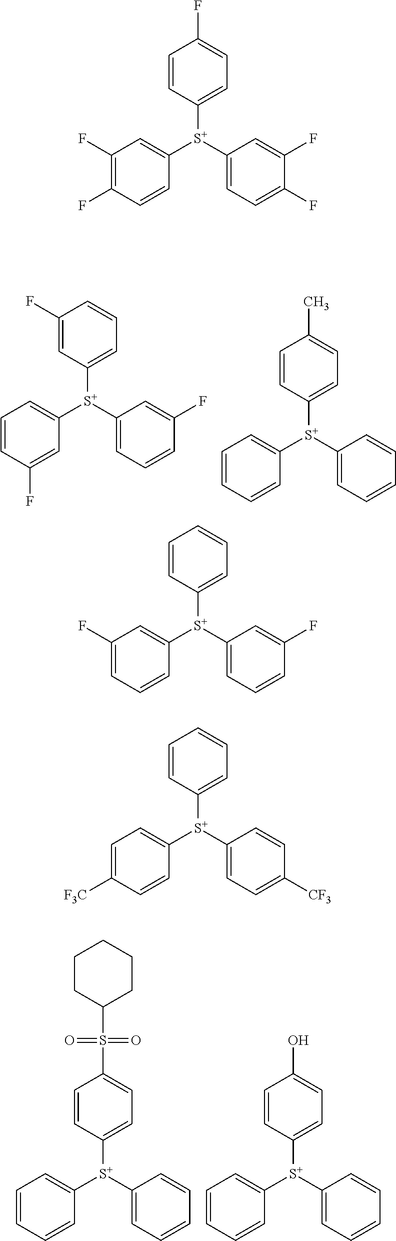

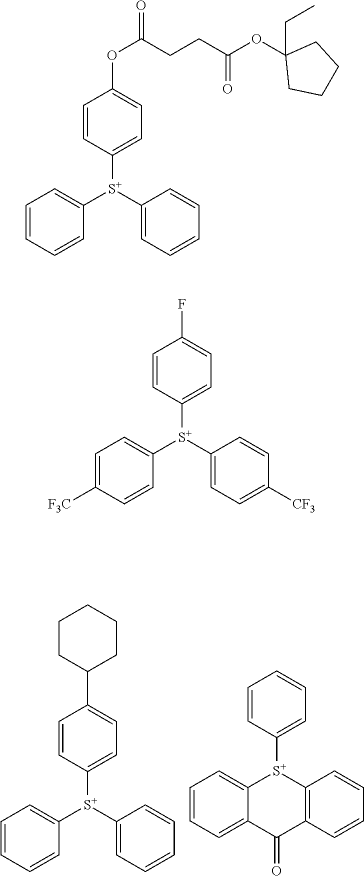 [SMILES: CCC1(OC(=O)CCC(=O)Oc2ccc([S+](c3ccccc3)c3ccccc3)cc2)CCCC1 ; Fc1ccc([S+](c2ccc(C(F)(F)F)cc2)c2ccc(C(F)(F)F)cc2)cc1 ; c1ccc([S+](c2ccccc2)c2ccc(C3CCCCC3)cc2)cc1 ; O=c1c2ccccc2[s+](-c2ccccc2)c2ccccc12]

Examples of the onium cation moiety of the formula (A-2) include those shown below. While all of those shown below are iodonium cation moieties having a fluorine-substituted aromatic ring structure, onium cation moieties having no fluorine-substituted aromatic ring structure that can be suitably employed include structures in which the fluorine atoms or CF₃ in the formulas shown below are replaced by an atom or group other than a fluorine atom such as a hydrogen atom or other substituent.

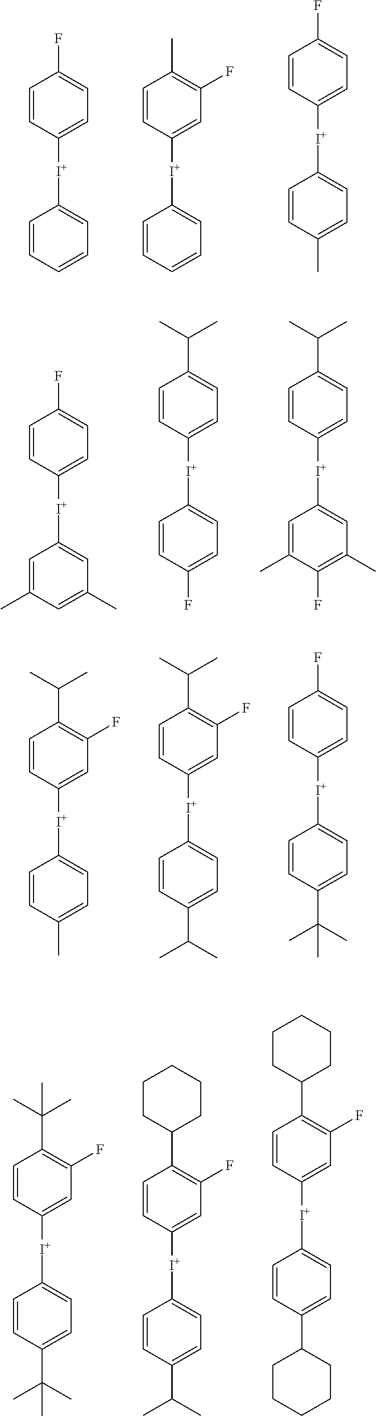

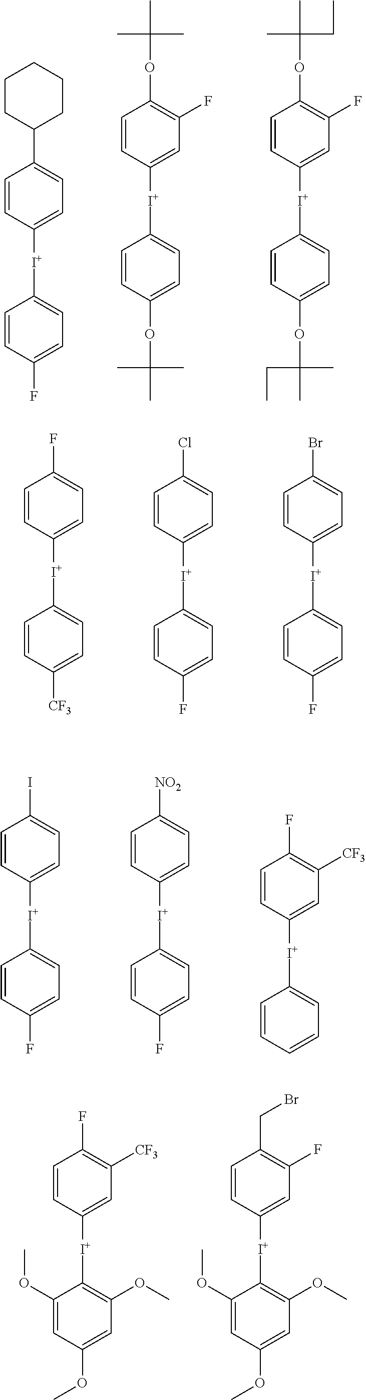

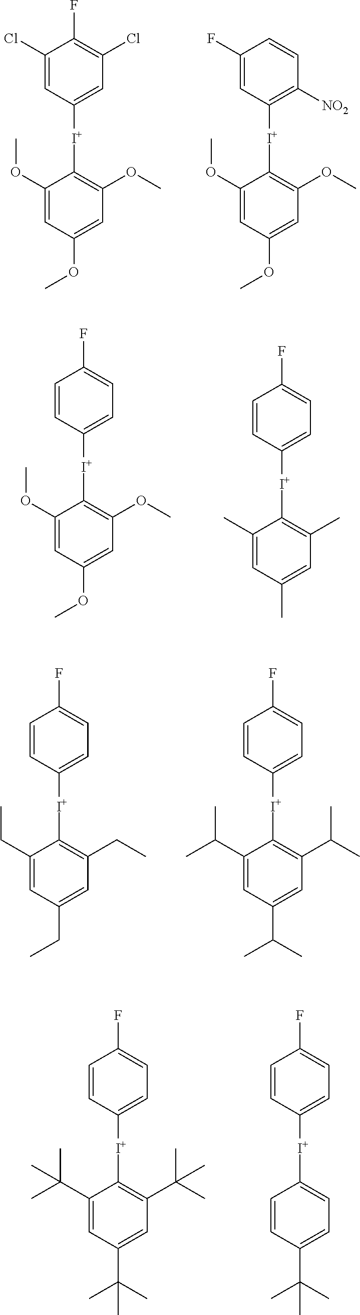

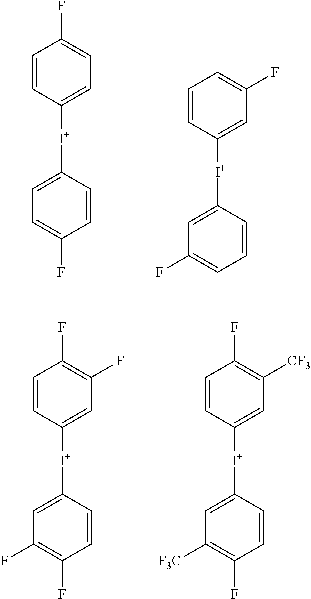 [SMILES: Fc1ccc([I+]c2ccc(F)cc2)cc1 ; Fc1cccc([I+]c2cccc(F)c2)c1 ; Fc1ccc([I+]c2ccc(F)c(F)c2)cc1F ; Fc1ccc([I+]c2ccc(F)c(C(F)(F)F)c2)cc1C(F)(F)F]

These radiation-sensitive acid generators may be used alone or in combination of two or more thereof. The content of the radiation-sensitive acid generator (when a plurality of types of radiation-sensitive acid generators are used, their total content is taken) is preferably 0.1 parts by mass or more, more preferably 1 part by mass or more, and still more preferably 5 parts by mass or more based on 100 parts by mass of the base resin). The content is preferably 50 parts by mass or less, and may be 45 parts by mass or less, 40 parts by mass or less, 35 parts by mass or less, 30 parts by mass or less, 25 parts by mass or less, 20 parts by mass or less, or the like based on 100 parts by mass of the resin. This makes it possible to exhibit excellent sensitivity, LWR performance, and CDU performance when forming a resist pattern.

### (Acid Diffusion Controlling Agent)

The acid diffusion controlling agent contains an organic acid anion moiety and an onium cation moiety and generates an acid having a higher pKa than an acid to be generated from the radiation-sensitive acid generator through irradiation with radiation. Examples of such an organic acid anion moiety include carboxylic acids. The organic acid anion moiety preferably contains an iodine-substituted aromatic ring structure. The acid diffusion controlling agent is preferably represented by the following formula (S-1) or (S-2).

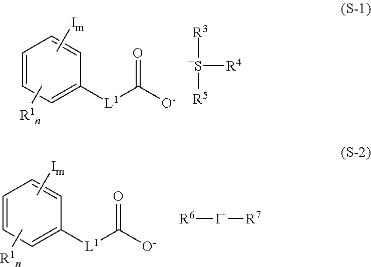

In the formulas (S-1) and (S-2), R¹ is a hydrogen atom, a hydroxy group, a fluorine atom, a chlorine atom, an amino group, a nitro group, or a cyano group; or an alkyl group having 1 to 6 carbon atoms, an alkoxy group having 1 to 6 carbon atoms, an acyloxy group having 2 to 6 carbon atoms, or an alkylsulfonyloxy group having 1 to 4 carbon atoms, which may be substituted with a halogen atom; or —NR^{1A}—C(═O)—R^{1B} or —NR^{1A}—C(═O)—O—R^{1B}. R^{1A} is a hydrogen atom or an alkyl group having 1 to 6 carbon atoms, and R^{1B} is an alkyl group having 1 to 6 carbon atoms or an alkenyl group having 2 to 8 carbon atoms.

Examples of the alkyl group having 1 to 6 carbon atoms include those having 1 to 6 carbon atoms among the chain hydrocarbon groups having 1 to 20 carbon atoms recited as examples for R².

In the formulas (S-1) and (S-2), R³, R⁴, R⁵, R⁶, and R⁷ are each independently a monovalent hydrocarbon group having 1 to 20 carbon atoms and optionally containing a heteroatom. When the onium cation moiety of the acid diffusion controlling agent has a fluorine atom, at least one of R³, R⁴, and R⁵ contains one or more fluorine atoms, and at least one of R⁶ and R⁷ contains one or more fluorine atoms. Any two of R³, R⁴, and R⁵ may be bonded to each other to form a ring together with the sulfur atom to which they are bonded. Examples of the monovalent hydrocarbon group include the same monovalent hydrocarbon groups as those recited as examples for the radiation-sensitive acid generator.

In the formulas (S-1) and (S-2), L¹ is a single bond or a divalent linking group having 1 to 20 carbon atoms, and may contain an ether linkage, a carbonyl group, an ester linkage, an amide linkage, a sultone ring, a lactam ring, a carbonate linkage, a halogen atom, a hydroxy group, or a carboxy group.

In the formulas (S-1) and (S-2), m and n are integers satisfying 0≤m≤5, 0≤n≤3, and 0≤m+n≤5, and preferably integers satisfying 1≤m≤3 and 0≤n≤2.

Examples of the organic acid anion moiety of the acid diffusion controlling agent represented by the above formula (S-1) or (S-2) include, but are not limited to, those shown below. While all of those shown below are organic acid anion moieties having an iodine-substituted aromatic ring structure, organic acid anion moieties having no iodine-substituted aromatic ring structure that can be suitably employed include structures in which the iodine atoms in the formulas shown below are replaced by an atom or group other than an iodine atom such as a hydrogen atom or other substituent.

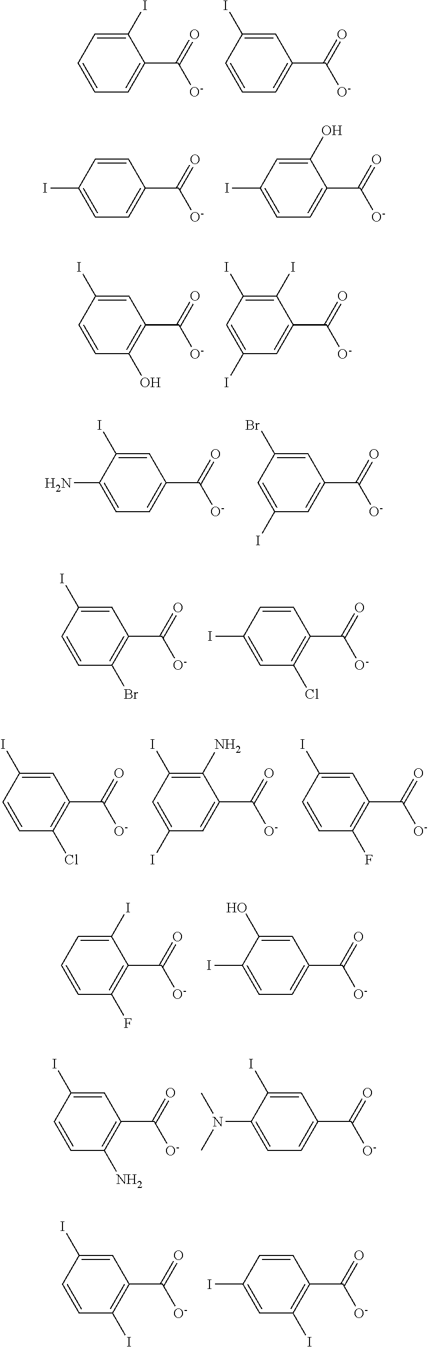

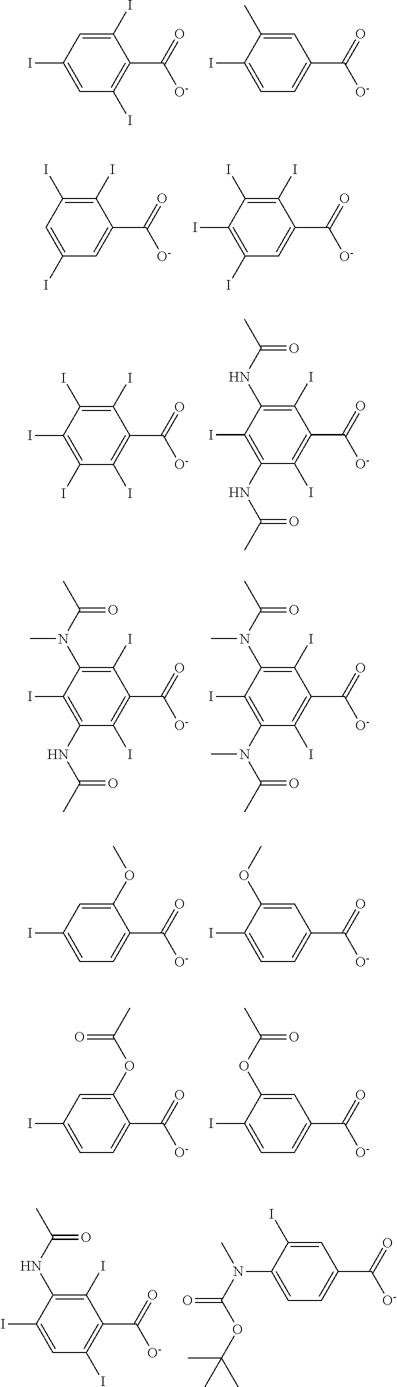

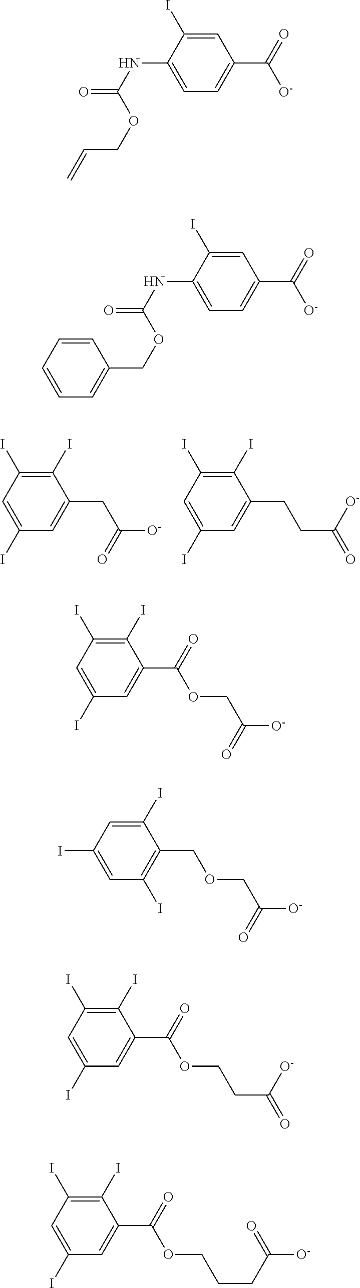

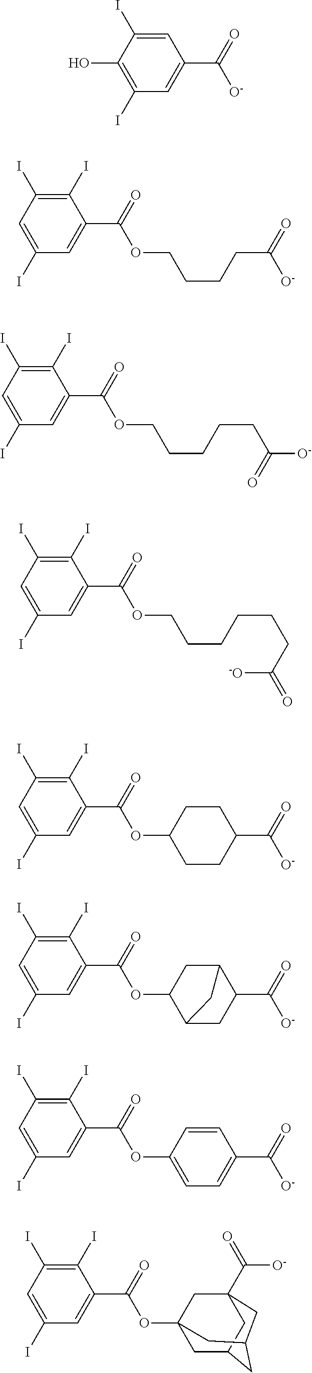

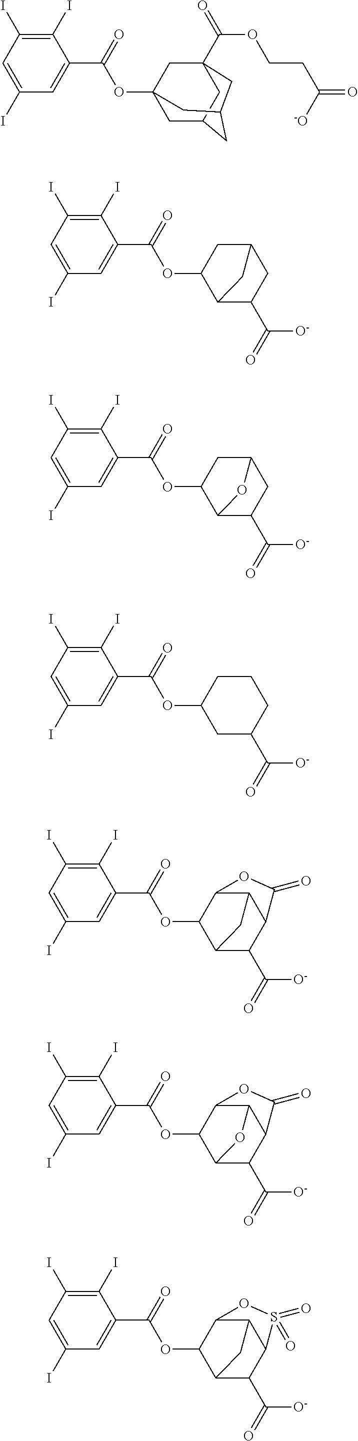

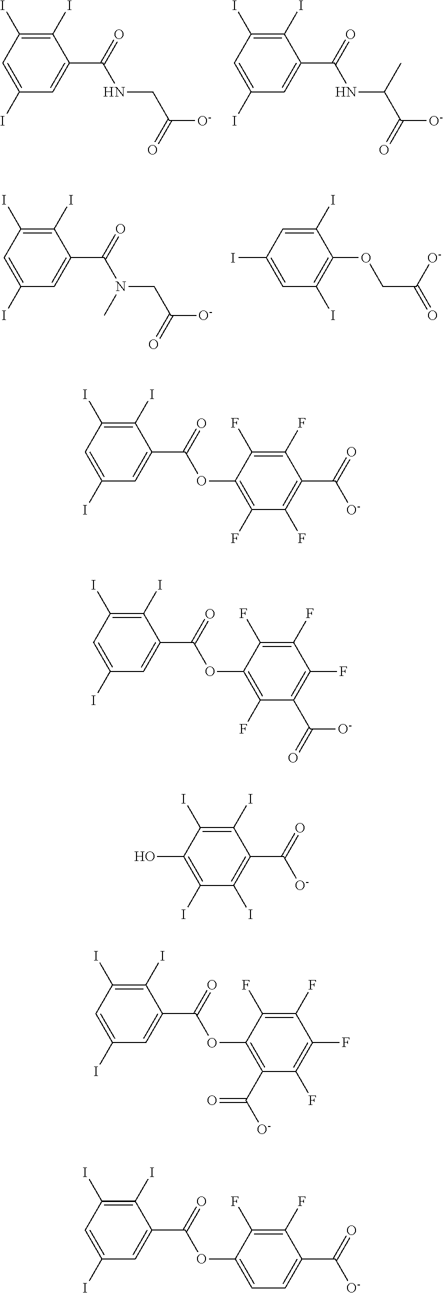

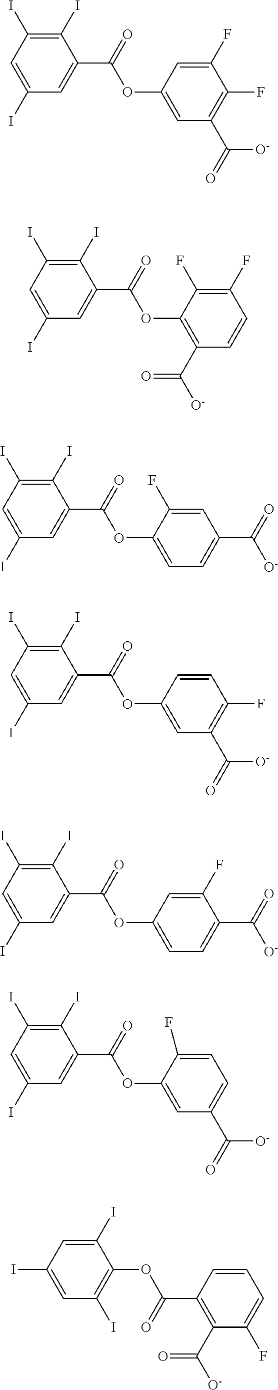

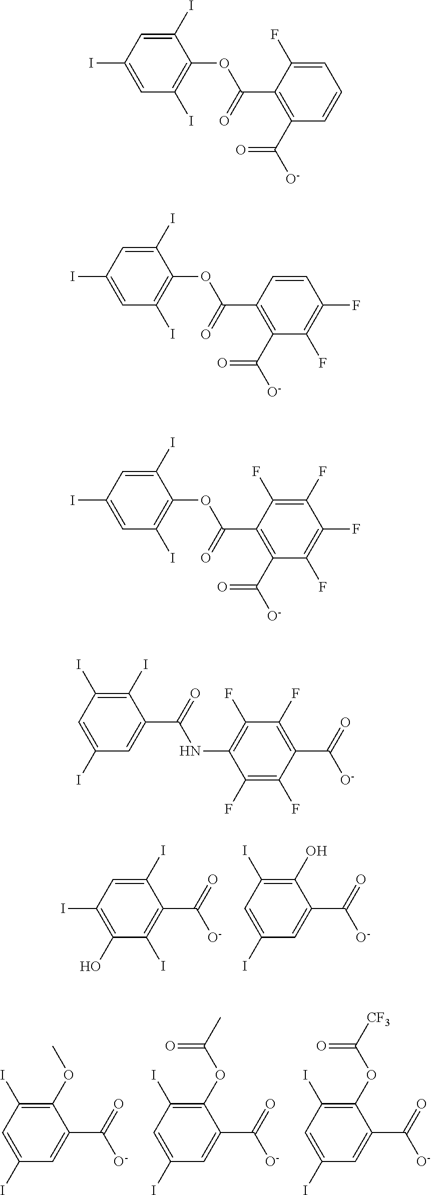

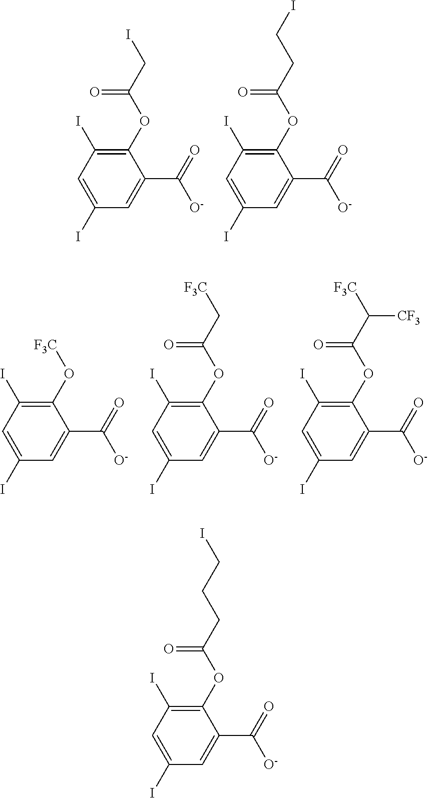 [SMILES: O=C(CI)Oc1c(I)cc(I)cc1C(=O)[O-] ; O=C(CCI)Oc1c(I)cc(I)cc1C(=O)[O-] ; O=C([O-])c1cc(I)cc(I)c1OC(F)(F)F ; O=C(CC(F)(F)F)Oc1c(I)cc(I)cc1C(=O)[O-] ; O=C([O-])c1cc(I)cc(I)c1OC(=O)C(C(F)(F)F)C(F)(F)F ; O=C(CCCI)Oc1c(I)cc(I)cc1C(=O)[O-]]

As the onium cation moieties in the acid diffusion controlling agents represented by the above formulas (S-1) and (S-2), onium cations containing an aromatic ring structure having a fluorine atom are preferable, and onium cations represented by the above formula (Q-1) are more preferable.

The acid diffusion controlling agents represented by the formulas (S-1) and (S-2) can also be synthesized by a known method, particularly by a salt exchange reaction. A known acid diffusion controlling agent may also be used as long as the effect of the present invention is not impaired.

These acid diffusion controlling agents may be used singly or two or more of them may be used in combination. The content of the acid diffusion controlling agent is preferably 10 mass % or more, more preferably 15 mass % or more, and still more preferably 20 mass % or more based on the content of the radiation-sensitive acid generator (when a radiation-sensitive acid generating resin is contained, the total amount with the content of the structural units a1 and a2 in 100 parts by mass of the radiation-sensitive acid generating resin is taken as the basis). The content is preferably 100 mass % or less, more preferably 80 mass % or less, and still more preferably 60 mass % or less. This makes it possible to exhibit superior sensitivity or CDU performance when forming a resist pattern.

The content of the acid diffusion controlling agent is preferably 5 mol % or more, more preferably 10 mol % or more, and still more preferably 15 mol % or more based on the total number of moles of the radiation-sensitive acid generator. The content is preferably 40 mol % or less, more preferably 30 mol % or less, and still more preferably 25 mol % or less based on the total number of moles of the radiation-sensitive acid generator. When the content of the acid diffusion controlling agent is set to fall within the above range, the lithographic performance of the radiation-sensitive resin composition can further be improved.

### (Solvent)

The radiation-sensitive resin composition according to the present embodiment contains a solvent. The solvent is not particularly limited as long as it is a solvent capable of dissolving or dispersing at least the resin having the structural unit (α), the radiation-sensitive acid generator, and the like.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
monoalcohol-based solvents having 1 to 18 carbon atoms, such as iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
polyhydric alcohol-based solvents having 2 to 18 carbon atoms, such as ethylene glycol, 1,2-propylene glycol, 2-methyl-2,4-pentanediol, 2,5-hexanediol, diethylene glycol, dipropylene glycol, triethylene glycol, and tripropylene glycol; and
partially etherized polyhydric alcohol-based solvents resulting from etherification of some of the hydroxy groups of the above-described polyhydric alcohol-based solvent.

Examples of the ether-based solvent include:
dialkyl ether-based solvents, such as diethyl ether, dipropyl ether, and dibutyl ether;
cyclic ether-based solvents, such as tetrahydrofuran and tetrahydropyran;
aromatic ring-containing ether-based solvents, such as diphenyl ether and anisole (methyl phenyl ether); and
polyhydric alcohol ether-based solvents resulting from etherification of the hydroxy groups of the above-described polyhydric alcohol-based solvent.

Examples of the ketone-based solvent include chain ketone-based solvents, such as acetone, butanone, and methyl-iso-butyl ketone;
cyclic ketone-based solvents, such as cyclopentanone, cyclohexanone, and methylcyclohexanone; and
2,4-pentanedione, acetonylacetone, and acetophenone.

Examples of the amide-based solvent include cyclic amide-based solvents, such as N,N′-dimethylimidazolidinone and N-methylpyrrolidone; and
cyclic amide-based solvents, such as N-methylformamide, N,N-dimethylformamide, N,N-diethylformamide, acetamide, N-methylacetamide, N,N-dimethylacetamide, and N-methylpropionamide.

Examples of the ester-based solvent include:
monocarboxylate ester-based solvents, such as n-butyl acetate and ethyl lactate;
partially etherized polyhydric alcohol acetate-based solvents, such as diethylene glycol mono-n-butyl ether acetate, propylene glycol monomethyl ether acetate, and dipropylene glycol monomethyl ether acetate;
lactone-based solvents, such as γ-butyrolactone and valerolactone;
carbonate-based solvents, such as diethyl carbonate, ethylene carbonate, and propylene carbonate; and
polyhydric carboxylic acid diester-based solvents, such as propylene glycol diacetate, methoxy triglycol acetate, diethyl oxalate, ethyl acetoacetate, ethyl lactate, and diethyl phthalate.

Examples of the hydrocarbon-based solvent include:
aliphatic hydrocarbon-based solvents, such as n-hexane, cyclohexane, and methylcyclohexane; and
aromatic hydrocarbon-based solvents, such as benzene, toluene, di-iso-propylbenzene, and n-amylnaphthalene.

Among them, ester-based solvents and ketone-based solvents are preferable, polyhydric alcohol partial ether acetate-based solvents, cyclic ketone-based solvents, and lactone-based solvents are more preferable, and propylene glycol monomethyl ether acetate, cyclohexanone, and γ-butyrolactone are still more preferable. The radiation-sensitive resin composition may contain one type or two or more types of solvent.

### (Other Optional Components)

The radiation-sensitive resin composition of the present disclosure may contain other optional components in addition to the components described above. Examples of the other optional components include a crosslinking agent, a localization enhancing agent, a surfactant, an alicyclic backbone-containing compound, and a sensitizer. Such other optional components may be used singly or two or more types thereof may be used in combination.

### <Method for Preparing Radiation-Sensitive Resin Composition>

The radiation-sensitive resin composition can be prepared, for example, by mixing a resin having the structural unit (α), a radiation-sensitive acid generator, and as necessary, a high fluorine-containing resin, a solvent, and the like in a prescribed ratio. The radiation-sensitive resin composition is preferably filtered through, for example, a filter having a pore size of about 0.20 μm after mixing. The solid concentration of the radiation-sensitive resin composition is usually from 0.1% by mass to 50% by mass, preferably from 0.5% by mass to 30% by mass, and more preferably from 1% by mass to 20% by mass.

### <Method for Forming Pattern Forming>

The method for forming a resist pattern according to one embodiment of the present disclosure comprises:
step (1) of applying the radiation-sensitive resin composition directly or indirectly to a substrate to form a resist film (hereinafter also referred to as “resist film formation step”),
step (2) of exposing the resist film to light (hereinafter also referred to as “exposure step”), and
step (3) of developing the exposed resist film (hereinafter also referred to as “development step”).

In accordance with this method for forming a resist pattern, a high-quality resist pattern can be formed because of the use of the radiation-sensitive resin composition superior in sensitivity, CDU performance, and pattern rectangularity in the exposure step. Hereinbelow, each of the steps will be described.

### [Resist Film Forming Step]

In this step (step (1)), a resist film is formed from the radiation-sensitive resin composition. Examples of the substrate on which the resist film is formed include conventionally known substrates such as a silicon wafer, silicon dioxide, and a wafer coated with aluminum. An organic or inorganic antireflective film disclosed in, for example, JP-B-6-12452 or JP-A-59-93448 may be formed on the substrate. Examples of a method for applying the composition include spin coating, cast coating, and roll coating. After the application, prebaking (PB) may be performed to volatilize the solvent in the coating film, as necessary. The PB temperature is usually 60° C. to 140° C., and preferably 80° C. to 120° C. The PB time is usually 5 seconds to 600 seconds, and preferably 10 seconds to 300 seconds. The thickness of the resist film to be formed is preferably 10 nm to 1,000 nm, and more preferably 10 nm to 500 nm.

When the subsequent exposure step is performed with radiation having a wavelength of 50 nm or less, it is preferable to use a resin having the structural unit (I) and the structural unit (IV) as the base resin in the composition.

### [Exposure Step]

In this step (the step (2)), the resist film formed in the resist film forming step, namely the step (1), is irradiated with radiation through a photomask (as the case may be, through an immersion medium such as water) to be exposed. Examples of the radiation to be used for the exposure include an electromagnetic wave including a visible ray, an ultraviolet ray, a far ultraviolet ray, an extreme ultraviolet ray (EUV), an X-ray, and a γ ray; an electron beam; and a charged particle radiation such as an a ray. Among them, far ultraviolet ray, electron beam, and EUV are preferable, ArF excimer laser light (wavelength: 193 nm), KrF excimer laser light (wavelength: 248 nm), electron beam, and EUV are more preferable, and an electron beam and EUV having a wavelength of 50 nm or less, which are positioned as next-generation exposure technology, are still more preferable.

After the exposure, post exposure baking (PEB) is preferably carried out to promote the dissociation of the acid-dissociable group of the resin or the like due to the acid generated from the radiation-sensitive acid generator through the exposure in the exposed area of the resist film. As a result of the PEB, there is produced a difference in solubility in the developer between the exposed area and the unexposed area. The PEB temperature is usually 50° C. to 180° C., and preferably 80° C. to 130° C. The PEB time is usually 5 seconds to 600 seconds, and preferably 10 seconds to 300 seconds.

### [Development Step]

In this step (the step (3)), the resist film exposed in the exposure step, namely the step (2), is developed. Thus, a prescribed resist pattern can be formed. In a common procedure, after the development, the film is washed with a rinsing liquid such as water or alcohol and dried.

Examples of the developer to be used for the development include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, and 1,5-diazabicyclo-[4.3.0]-5-nonene. Among them, the aqueous TMAH solution is preferable, and a 2.38% by mass aqueous TMAH solution is more preferable.

In the case of organic solvent development, examples of the solvent include organic solvents such as hydrocarbon-based solvents, ether-based solvents, ester-based solvents, ketone-based solvents, and alcohol-based solvents, and solvents containing an organic solvent. Examples of the organic solvent include one type or two or more types of solvent among the solvents listed as the solvent for the radiation-sensitive resin composition. Among them, the ether-based solvent, the ester-based solvent, and the ketone-based solvent are preferable.

As described above, the developer may be either an alkaline developer or an organic solvent developer.

Examples of a development method include a method in which a substrate is immersed in a bath filled with a developer for a certain period of time (dipping method), a method in which a developer is allowed to be present on a surface of a substrate due to surface tension and to stand for a certain period of time (puddle method), a method in which a developer is sprayed onto a surface of a substrate (spray method), and a method in which a developer is discharged onto a substrate that is rotated at a constant speed while a developer discharge nozzle is scanned at a constant speed (dynamic dispensing method).

### <Polymer>

The polymer of the present disclosure is a polymer having a structural unit represented by the above formula (1) (structural unit (α)).

The polymer may be a resin having the structural unit (α) described above, and R¹, L¹, L, R², L², Ar, R³, n, and the like follow the description on the resin having the structural unit (α) described above, and the like.

The polymer (2) of the present disclosure is a polymer having a structural unit represented by the above formula (2) (structural unit (β)).

The polymer (2) may be a resin having the structural unit (β) described above, and R¹, L¹, L, R², L², m, R³, p, and the like follow the description on the resin having the structural unit (β) described above, and the like.

The polymer (3) of the present disclosure is a polymer having a structural unit represented by the above formula (3) (structural unit (γ)).

The polymer (3) may be a resin having the structural unit (γ) described above, and R¹, L¹, L, R², L², X, R³, o, and the like follow the description on the resin having the structural unit (γ) described above, and the like.

### <Compound>

The compound of the present disclosure is a compound represented by the following formula (7),

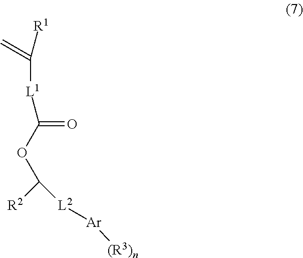

in the formula (7),
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
Ar represents a group obtained by removing (n+1) hydrogen atoms from an aromatic ring;
R³ is each independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group;
n is an integer of 1 or more; and when n is 2 or more, a plurality of R³'s are same or different from each other.

In the formula (7), R¹, L¹, L, R², L², Ar, R³, n, and the like follow the description on the resin having the structural unit (α) described above, and the like.

Examples of the compound (7) include compounds represented by the above formulas (M-1) to (M-32).

The compound (5) of the present disclosure is a compound represented by the above formula (5).

In the formula (5), R¹, L¹, L, R², L², m, R³, p, and the like follow the description on the resin having the structural unit (β) described above, and the like.

The compound (6) of the present disclosure is a compound represented by the above formula (6).

In the formula (6), R¹, L¹, L, R², L², X, R³, o, and the like follow the description on the resin having the structural unit (γ) described above, and the like.

### EXAMPLES

Hereinafter, the present invention will be specifically described with reference to Examples, but the present invention is not limited to these Examples. Methods for measuring various physical property values will be described below.

### [Measurement of Weight Average Molecular Weight (Mw), Number Average Molecular Weight (Mn), and Dispersity (Mw/Mn)]

The weight average molecular weight (Mw), the number average molecular weight (Mn), and the degree of dispersion (Mw/Mn) were measured by gel permeation chromatography (GPC) under the following conditions.

GPC column: two G2000HXL, one G3000HXL, one G4000HXL (all manufactured by Tosoh Corporation)
Column temperature: 40° C.
Elution solvent: tetrahydrofuran
Flow rate: 1.0 mL/min
Sample concentration: 1.0% by mass
Amount of sample injected: 100 μL
Detector: differential refractometer
Standard substance: monodisperse polystyrene
   [¹H-NMR Analysis and ¹³C-NMR Analysis]

¹H-NMR analysis and ¹³C-NMR analysis were performed using a nuclear magnetic resonance apparatus (“JNM-Delta 400” manufactured by JEOL Ltd.).

### <Synthesis of Polymer>

The monomers used for the synthesis of the respective polymers in the respective Examples and Comparative Examples are shown below. In the following synthesis examples, unless otherwise specified, “parts by mass” means a value taken when the total mass of the monomers used is 100 parts by mass, and “mol %” means a value taken when the total number of moles of the monomers used is 100 mol %. In addition, the present invention is not limited to the following structural units.

Among the monomers used in the synthesis of the polymers in Examples, the structures of the monomers to afford the structural units represented by the formula (1) are shown below.

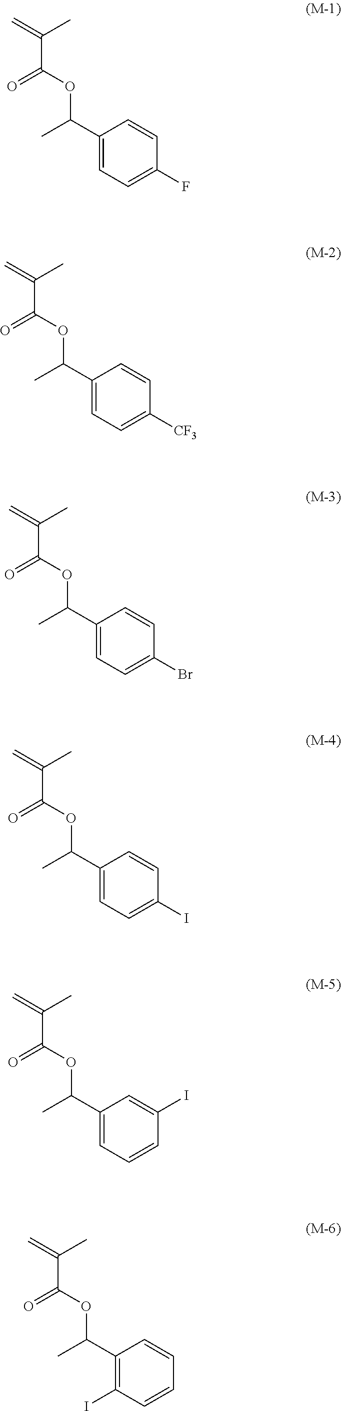

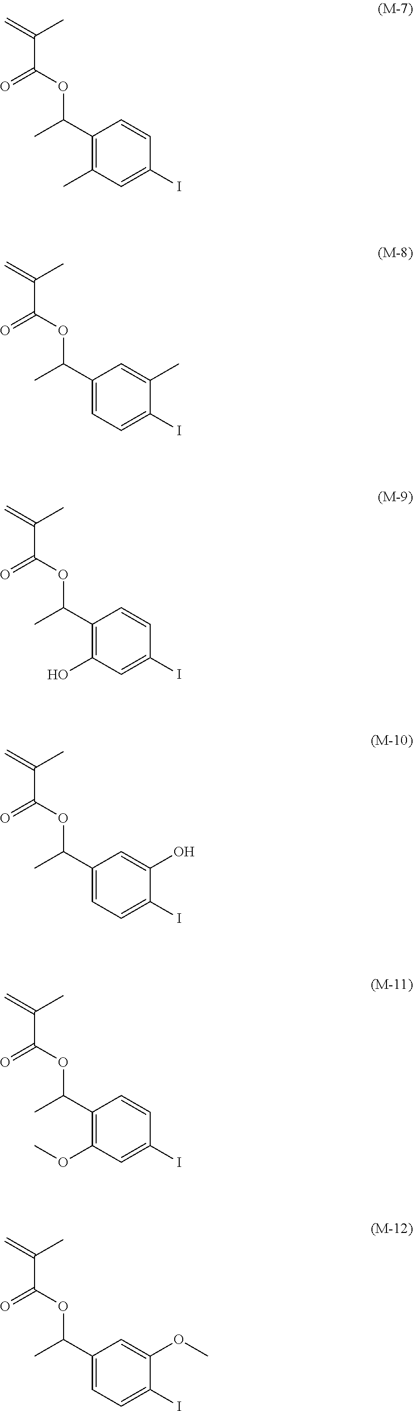

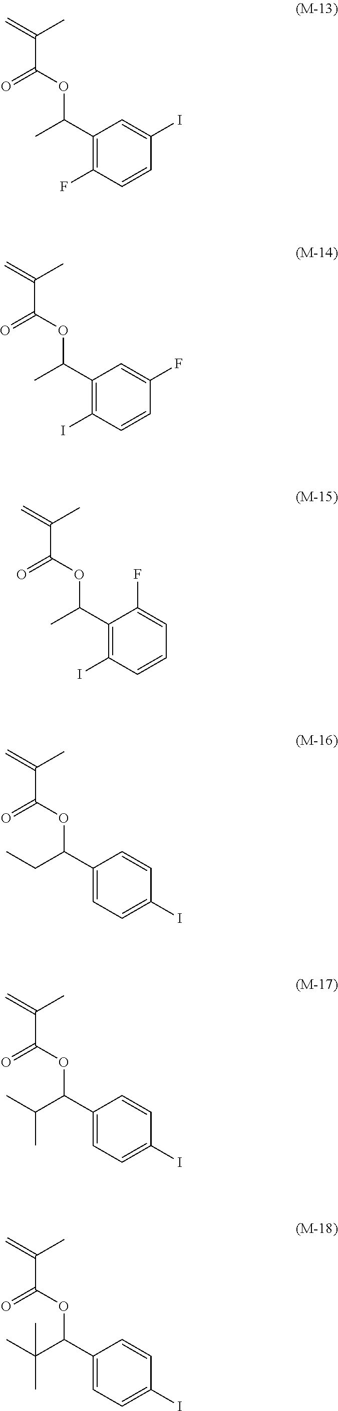

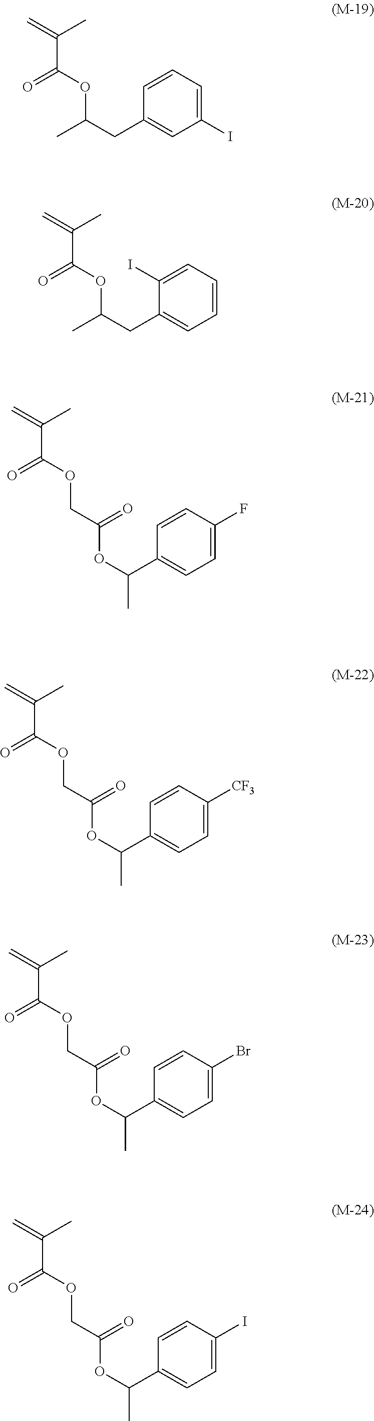

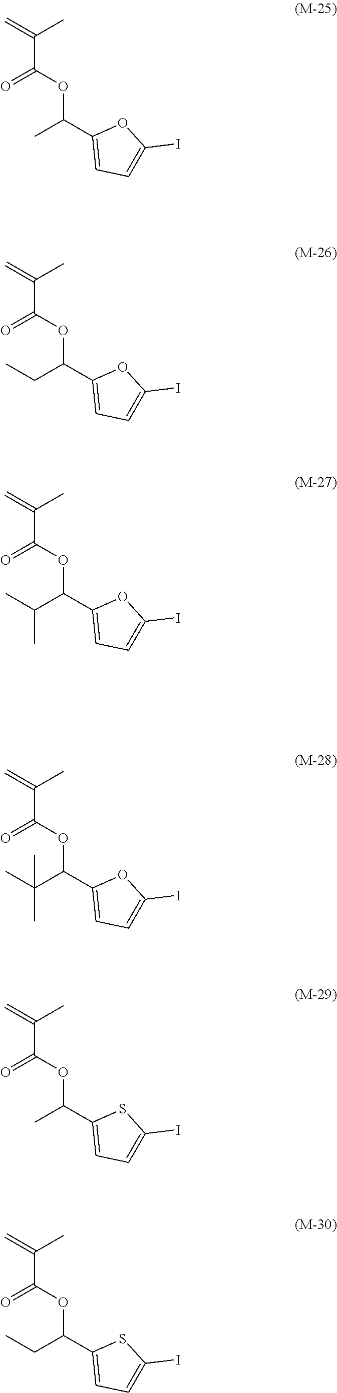

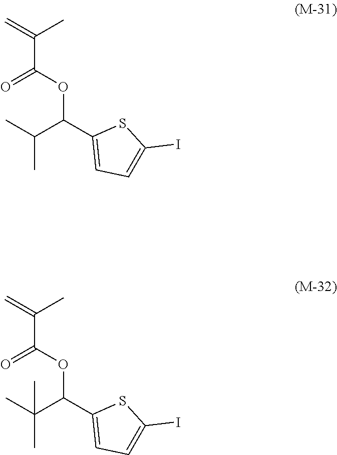

Among the monomers used in the synthesis of the polymers in Examples and Comparative Examples, the structures of the monomers other than the above are shown.

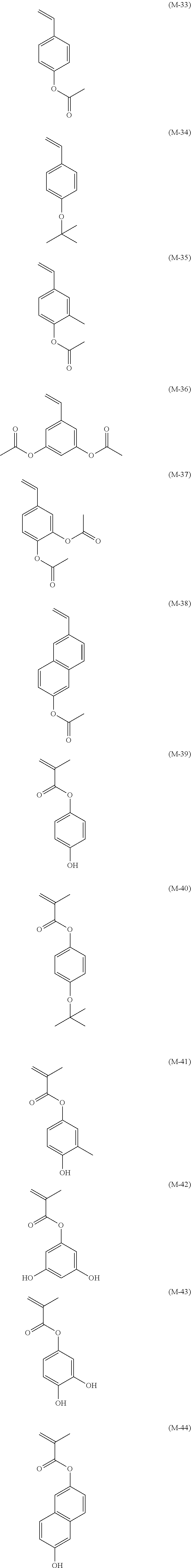

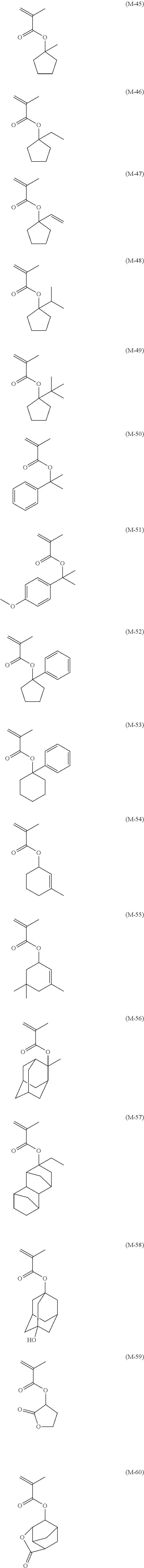

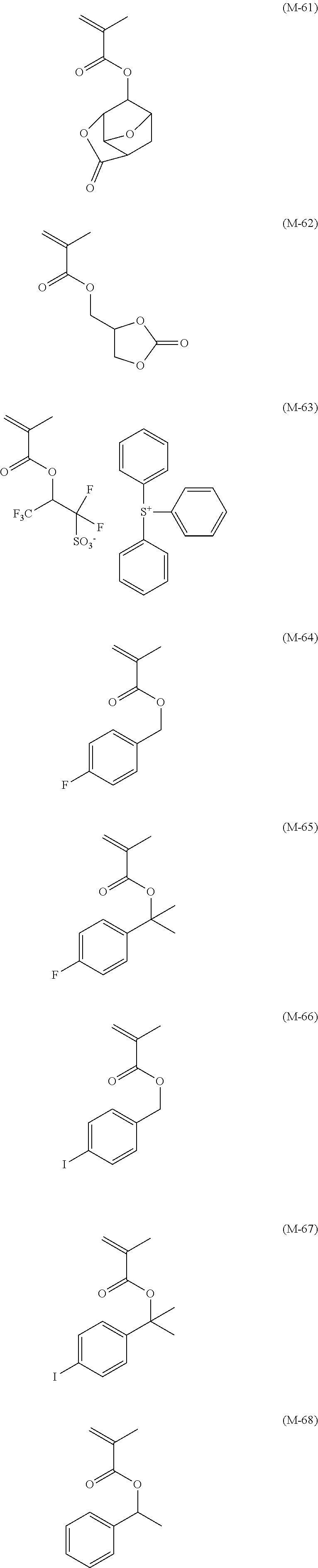

### [Method for Synthesizing Monomer to Afford Structural Unit Represented by Formula (1)]

The monomers (M-1) to (M-32) that afford the structural unit represented by the formula (1) can be synthesized in the same manner as the method for synthesizing the monomer M-1.

### [Monomer Synthesis Example 1] (Synthesis of Monomer M-1)

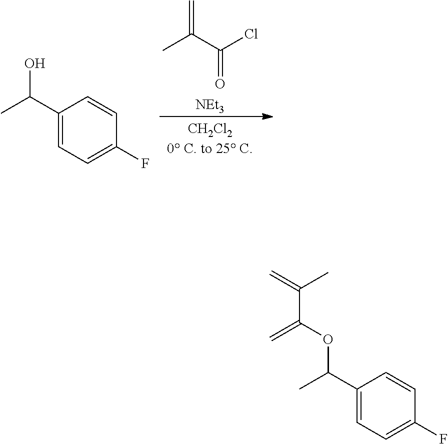

200 g (1.43 mol) of 1-(4-fluorophenyl)ethanol and 217 g (2.14 mol) of triethylamine were dissolved in dichloromethane (1,500 mL). A solution was cooled to 0° C., and then 149 g (1.43 mol) of methacryloyl chloride was added dropwise such that the temperature of the solution did not exceed 25° C. After the completion of the dropwise addition, the mixture was stirred at 25° C. for 1 hour. After the completion of the reaction, the reaction mixture was quenched with a saturated aqueous solution of ammonium chloride, and extracted with methylene chloride. The residue obtained by concentration under reduced pressure was purified by column chromatography to obtain 224 g (yield: 76%) of a monomer (M-1).

### [Method for Synthesizing Polymer]

### [Polymer Synthesis Example 1] Synthesis of Polymer (A-1)

Compound (M-1) and compound (M-33) were dissolved in 1-methoxy-2-propanol (200 parts by mass based on the total amount of the monomers) so as to have a molar ratio of 40/60. Next, azobisisobutyronitrile (AIBN) was added as an initiator in an amount of 6 mol % based on all the monomers to prepare a monomer solution. On the other hand, 1-methoxy-2-propanol (100 parts by mass based on the total amount of monomers) was added to an empty reaction vessel, and was heated to 85° C. with stirring. Next, the monomer solution prepared above was added dropwise over 3 hours, and then the mixture was heated at 85° C. for another 3 hours to perform a polymerization reaction for 6 hours in total. After the completion of the polymerization reaction, the polymerization solution was cooled to room temperature.

The cooled polymerization solution was charged into hexane (500 parts by mass based on the polymerization solution), and a precipitated white powder was separated by filtration. The white powder separated by filtration was washed twice with 100 parts by mass of hexane relative to the polymerization solution, and dissolved again in 1-methoxy-2-propanol (300 parts by mass). Next, methanol (500 parts by mass), triethylamine (50 parts by mass) and ultrapure water (10 parts by mass) were added, and a hydrolysis reaction was performed at 70° C. for 6 hours with stirring.

After the completion of the reaction, the remaining solvent was distilled off, and the resulting solid was dissolved in acetone (100 parts by mass). The resulting solution was added dropwise into 500 parts by mass of water to permit the coagulation of the resin. The resulting solid was separated by filtration. By drying at 50° C. for 12 hours, a white powdery polymer (A-1) was synthesized.

### Polymer Synthesis Examples 2 to 63 and 70 to 77

Synthesis of Polymers (A-2) to (A-63) and (A-69) to (A-76)

Polymers (A-2) to (A-63) and (A-69) to (A-76) were obtained in the same manner as in Polymer Synthesis Example 1 except that the monomers of the types shown in Table 1 were blended in the prescribed amounts. Mw and Mw/Mn of the respective polymers obtained are shown in Table 1.

### [Polymer Synthesis Example 65 to 69] Synthesis of Polymers (A-64) to (A-68)

Polymers (A-64) to (A-68) were obtained in the same manner as in Polymer Synthesis Example 2 except that the amount of the initiator was appropriately changed. Mw and Mw/Mn of the respective polymers obtained are shown in Table 1.

### [Polymer Synthesis Example 64] Synthesis of Polymer (B-1)

Compound (M-4), compound (M-39), and compound (M-63) were dissolved in 2-butanone (200 parts by mass based on the total amount of the monomers) so as to have a molar ratio of 50/40/10. 6 mol % of AIBN as an initiator was added into the total of the monomers to prepare a monomer solution. Meanwhile, 2-butanone (100 parts by mass) was added to an empty reaction vessel, and was heated to 80° C. while being stirred. Next, the monomer solution prepared above was added dropwise over 3 hours. Thereafter, the mixture was further heated at 80° C. for 3 hours to perform a polymerization reaction for 6 hours in total. After the completion of the polymerization reaction, the polymerization solution was cooled to room temperature.

The cooled polymerization solution was charged into methanol (2,000 parts by mass with respect to the polymerization solution), and a precipitated white powder was separated by filtration. The solid obtained was dissolved in acetone (100 parts by mass), and the solution was added dropwise to 500 parts by mass of water. The resulting solid was separated by filtration, and dried at 50° C. for 12 hours, affording a white powdery polymer (B-1). Mw and Mw/Mn of the polymer obtained are shown in Table 1.

TABLE 1

| | | Structural unit | | | | | | Structural unit | | | |
|---|---|---|---|---|---|---|---|---|---|---|---|
| | | (I) | | Structural unit (II) | | | | (III) | | Physical | |
| | | | Amount | | Amount | | Amount | | Amount | property value | |
| | Polymer | Type | used | Type | used | Type | used | Type | used | Mw | Mw/Mn |
| Polymer Synthesis | A-1 | M-1 | 40 | M-33 | 60 | — | — | — | — | 8100 | 1.6 |
| Example 1 | | | | | | | | | | | |
| Polymer Synthesis | A-2 | M-1 | 50 | M-33 | 50 | — | — | — | — | 7900 | 1.6 |
| Example 2 | | | | | | | | | | | |
| Polymer Synthesis | A-3 | M-1 | 60 | M-33 | 40 | — | — | — | — | 8000 | 1.6 |
| Example 3 | | | | | | | | | | | |
| Polymer Synthesis | A-4 | M-2 | 50 | M-33 | 50 | — | — | — | — | 7500 | 1.5 |
| Example 4 | | | | | | | | | | | |
| Polymer Synthesis | A-5 | M-3 | 50 | M-33 | 50 | — | — | — | — | 7100 | 1.5 |
| Example 5 | | | | | | | | | | | |
| Polymer Synthesis | A-6 | M-4 | 50 | M-33 | 50 | — | — | — | — | 8200 | 1.6 |
| Example 6 | | | | | | | | | | | |
| Polymer Synthesis | A-7 | M-5 | 50 | M-33 | 50 | — | — | — | — | 7900 | 1.6 |
| Example 7 | | | | | | | | | | | |
| Polymer Synthesis | A-8 | M-6 | 50 | M-33 | 50 | — | — | — | — | 7700 | 1.5 |
| Example 8 | | | | | | | | | | | |
| Polymer Synthesis | A-9 | M-7 | 50 | M-33 | 50 | — | — | — | — | 8100 | 1.6 |
| Example 9 | | | | | | | | | | | |
| Polymer Synthesis | A-10 | M-8 | 50 | M-33 | 50 | — | — | — | — | 7000 | 1.6 |
| Example 10 | | | | | | | | | | | |
| Polymer Synthesis | A-11 | M-9 | 50 | M-33 | 50 | — | — | — | — | 7600 | 1.6 |
| Example 11 | | | | | | | | | | | |
| Polymer Synthesis | A-12 | M-10 | 50 | M-33 | 50 | — | — | — | — | 7800 | 1.6 |
| Example 12 | | | | | | | | | | | |
| Polymer Synthesis | A-13 | M-11 | 50 | M-33 | 50 | — | — | — | — | 8000 | 1.6 |
| Example 13 | | | | | | | | | | | |
| Polymer Synthesis | A-14 | M-12 | 50 | M-33 | 50 | — | — | — | — | 7200 | 1.5 |
| Example 14 | | | | | | | | | | | |
| Polymer Synthesis | A-15 | M-13 | 50 | M-33 | 50 | — | — | — | — | 7400 | 1.6 |
| Example 15 | | | | | | | | | | | |
| Polymer Synthesis | A-16 | M-14 | 50 | M-33 | 50 | — | — | — | — | 7800 | 1.6 |
| Example 16 | | | | | | | | | | | |
| Polymer Synthesis | A-17 | M-15 | 50 | M-33 | 50 | — | — | — | — | 7300 | 1.7 |
| Example 17 | | | | | | | | | | | |
| Polymer Synthesis | A-18 | M-16 | 50 | M-33 | 50 | — | — | — | — | 8000 | 1.5 |
| Example 18 | | | | | | | | | | | |
| Polymer Synthesis | A-19 | M-17 | 50 | M-33 | 50 | — | — | — | — | 8100 | 1.4 |
| Example 19 | | | | | | | | | | | |
| Polymer Synthesis | A-20 | M-18 | 50 | M-33 | 50 | — | — | — | — | 7400 | 1.5 |
| Example 20 | | | | | | | | | | | |
| Polymer Synthesis | A-21 | M-19 | 50 | M-33 | 50 | — | — | — | — | 7700 | 1.5 |
| Example 21 | | | | | | | | | | | |
| Polymer Synthesis | A-22 | M-20 | 50 | M-33 | 50 | — | — | — | — | 7500 | 1.4 |
| Example 22 | | | | | | | | | | | |
| Polymer Synthesis | A-23 | M-21 | 50 | M-33 | 50 | — | — | — | — | 7100 | 1.7 |
| Example 23 | | | | | | | | | | | |
| Polymer Synthesis | A-24 | M-22 | 50 | M-33 | 50 | — | — | — | — | 7600 | 1.6 |
| Example 24 | | | | | | | | | | | |
| Polymer Synthesis | A-25 | M-23 | 50 | M-33 | 50 | — | — | — | — | 7900 | 1.6 |
| Example 25 | | | | | | | | | | | |
| Polymer Synthesis | A-26 | M-24 | 50 | M-33 | 50 | — | — | — | — | 7500 | 1.5 |
| Example 26 | | | | | | | | | | | |
| Polymer Synthesis | A-27 | M-25 | 50 | M-33 | 50 | — | — | — | — | 7100 | 1.5 |
| Example 27 | | | | | | | | | | | |
| Polymer Synthesis | A-28 | M-26 | 50 | M-33 | 50 | — | — | — | — | 7700 | 1.6 |
| Example 28 | | | | | | | | | | | |
| Polymer Synthesis | A-29 | M-27 | 50 | M-33 | 50 | — | — | — | — | 7600 | 1.6 |
| Example 29 | | | | | | | | | | | |
| Polymer Synthesis | A-30 | M-28 | 50 | M-33 | 50 | — | — | — | — | 7200 | 1.5 |
| Example 30 | | | | | | | | | | | |
| Polymer Synthesis | A-31 | M-29 | 50 | M-33 | 50 | — | — | — | — | 7400 | 1.5 |
| Example 31 | | | | | | | | | | | |
| Polymer Synthesis | A-32 | M-30 | 50 | M-33 | 50 | — | — | — | — | 7900 | 1.5 |
| Example 32 | | | | | | | | | | | |
| Polymer Synthesis | A-33 | M-31 | 50 | M-33 | 50 | — | — | — | — | 7300 | 1.6 |
| Example 33 | | | | | | | | | | | |
| Polymer Synthesis | A-34 | M-32 | 50 | M-33 | 50 | — | — | — | — | 8200 | 1.6 |
| Example 34 | | | | | | | | | | | |
| Polymer Synthesis | A-35 | M-4 | 50 | M-33 | 25 | M-34 | 25 | — | — | 7600 | 1.5 |
| Example 35 | | | | | | | | | | | |
| Polymer Synthesis | A-36 | M-4 | 50 | M-33 | 25 | M-35 | 25 | — | — | 7600 | 1.6 |
| Example 36 | | | | | | | | | | | |
| Polymer Synthesis | A-37 | M-4 | 50 | M-33 | 25 | M-36 | 25 | — | — | 7800 | 1.5 |
| Example 37 | | | | | | | | | | | |
| Polymer Synthesis | A-38 | M-4 | 50 | M-33 | 25 | M-37 | 25 | — | — | 7800 | 1.6 |
| Example 38 | | | | | | | | | | | |
| Polymer Synthesis | A-39 | M-4 | 50 | M-33 | 25 | M-38 | 25 | — | — | 7100 | 1.6 |
| Example 39 | | | | | | | | | | | |
| Polymer Synthesis | A-40 | M-4 | 50 | M-33 | 25 | M-39 | 25 | — | — | 7900 | 1.6 |
| Example 40 | | | | | | | | | | | |
| Polymer Synthesis | A-41 | M-4 | 25 | M-33 | 50 | M-40 | 25 | — | — | 8100 | 1.6 |
| Example 41 | | | | | | | | | | | |
| Polymer Synthesis | A-42 | M-4 | 50 | M-33 | 25 | M-41 | 25 | — | — | 7300 | 1.5 |
| Example 42 | | | | | | | | | | | |
| Polymer Synthesis | A-43 | M-4 | 50 | M-33 | 25 | M-42 | 25 | — | — | 7400 | 1.6 |
| Example 43 | | | | | | | | | | | |
| Polymer Synthesis | A-44 | M-4 | 50 | M-33 | 25 | M-43 | 25 | — | — | 7900 | 1.6 |
| Example 44 | | | | | | | | | | | |
| Polymer Synthesis | A-45 | M-4 | 50 | M-33 | 25 | M-44 | 25 | — | — | 7200 | 1.5 |
| Example 45 | | | | | | | | | | | |
| Polymer Synthesis | A-46 | M-4 | 25 | M-33 | 50 | — | — | M-45 | 25 | 7400 | 1.5 |
| Example 46 | | | | | | | | | | | |
| Polymer Synthesis | A-47 | M-4 | 25 | M-33 | 50 | — | — | M-46 | 25 | 7600 | 1.5 |
| Example 47 | | | | | | | | | | | |
| Polymer Synthesis | A-48 | M-4 | 25 | M-33 | 50 | — | — | M-47 | 25 | 7300 | 1.7 |
| Example 48 | | | | | | | | | | | |
| Polymer Synthesis | A-49 | M-4 | 25 | M-33 | 50 | — | — | M-48 | 25 | 8200 | 1.6 |
| Example 49 | | | | | | | | | | | |
| Polymer Synthesis | A-50 | M-4 | 25 | M-33 | 50 | — | — | M-49 | 25 | 7900 | 1.6 |
| Example 50 | | | | | | | | | | | |
| Polymer Synthesis | A-51 | M-4 | 25 | M-33 | 50 | — | — | M-50 | 25 | 7800 | 1.5 |
| Example 51 | | | | | | | | | | | |
| Polymer Synthesis | A-52 | M-4 | 25 | M-33 | 50 | — | — | M-51 | 25 | 8100 | 1.5 |
| Example 52 | | | | | | | | | | | |
| Polymer Synthesis | A-53 | M-4 | 25 | M-33 | 50 | — | — | M-52 | 25 | 8300 | 1.6 |
| Example 53 | | | | | | | | | | | |
| Polymer Synthesis | A-54 | M-4 | 25 | M-33 | 50 | — | — | M-53 | 25 | 7500 | 1.5 |
| Example 54 | | | | | | | | | | | |
| Polymer Synthesis | A-55 | M-4 | 25 | M-33 | 50 | — | — | M-54 | 25 | 7100 | 1.7 |
| Example 55 | | | | | | | | | | | |
| Polymer Synthesis | A-56 | M-4 | 25 | M-33 | 50 | — | — | M-55 | 25 | 8200 | 1.6 |
| Example 56 | | | | | | | | | | | |
| Polymer Synthesis | A-57 | M-4 | 25 | M-33 | 50 | — | — | M-56 | 25 | 7900 | 1.5 |
| Example 57 | | | | | | | | | | | |
| Polymer Synthesis | A-58 | M-4 | 25 | M-33 | 50 | — | — | M-57 | 25 | 7600 | 1.6 |
| Example 58 | | | | | | | | | | | |
| Polymer Synthesis | A-59 | M-4 | 30 | M-33 | 50 | — | — | M-58 | 20 | 7300 | 1.6 |
| Example 59 | | | | | | | | | | | |
| Polymer Synthesis | A-60 | M-4 | 30 | M-33 | 50 | — | — | M-59 | 20 | 7900 | 1.6 |
| Example 60 | | | | | | | | | | | |
| Polymer Synthesis | A-61 | M-4 | 30 | M-33 | 50 | — | — | M-60 | 20 | 7700 | 1.6 |
| Example 61 | | | | | | | | | | | |
| Polymer Synthesis | A-62 | M-4 | 30 | M-33 | 50 | — | — | M-61 | 20 | 8100 | 1.5 |
| Example 62 | | | | | | | | | | | |
| Polymer Synthesis | A-63 | M-4 | 30 | M-33 | 50 | — | — | M-62 | 20 | 7600 | 1.6 |
| Example 63 | | | | | | | | | | | |
| Polymer Synthesis | B-1 | M-4 | 50 | — | — | M-39 | 40 | M-63 | 10 | 8500 | 1.7 |
| Example 64 | | | | | | | | | | | |
| Polymer Synthesis | A-64 | M-1 | 50 | M-33 | 50 | — | — | — | — | 19800 | 1.8 |
| Example 65 | | | | | | | | | | | |
| Polymer Synthesis | A-65 | M-1 | 50 | M-33 | 50 | — | — | — | — | 14700 | 1.7 |
| Example 66 | | | | | | | | | | | |
| Polymer Synthesis | A-66 | M-1 | 50 | M-33 | 50 | — | — | — | — | 10200 | 1.6 |
| Example 67 | | | | | | | | | | | |
| Polymer Synthesis | A-67 | M-1 | 50 | M-33 | 50 | — | — | — | — | 5200 | 1.5 |
| Example 68 | | | | | | | | | | | |
| Polymer Synthesis | A-68 | M-1 | 50 | M-33 | 50 | — | — | — | — | 3500 | 1.5 |
| Example 69 | | | | | | | | | | | |
| Polymer Synthesis | A-69 | — | — | M-33 | 50 | — | — | M-45 | 50 | 7500 | 1.5 |
| Example 70 | | | | | | | | | | | |
| Polymer Synthesis | A-70 | — | — | M-33 | 50 | — | — | M-46 | 50 | 7100 | 1.5 |
| Example 71 | | | | | | | | | | | |
| Polymer Synthesis | A-71 | — | — | M-33 | 50 | — | — | M-47 | 50 | 7600 | 1.6 |
| Example 72 | | | | | | | | | | | |
| Polymer Synthesis | A-72 | — | — | M-33 | 50 | M-45 | 25 | M-64 | 25 | 7200 | 1.6 |
| Example 73 | | | | | | | | | | | |
| Polymer Synthesis | A-73 | — | — | M-33 | 50 | — | — | M-65 | 50 | 7400 | 1.5 |
| Example 74 | | | | | | | | | | | |
| Polymer Synthesis | A-74 | — | — | M-33 | 50 | M-45 | 25 | M-66 | 25 | 7900 | 1.5 |
| Example 75 | | | | | | | | | | | |
| Polymer Synthesis | A-75 | — | — | M-33 | 50 | — | — | M-67 | 50 | 8200 | 1.6 |
| Example 76 | | | | | | | | | | | |
| Polymer Synthesis | A-76 | — | — | M-33 | 50 | — | — | M-68 | 50 | 8200 | 1.6 |
| Example 77 | | | | | | | | | | | |

### <Preparation of Radiation-Sensitive Resin Composition>

The radiation-sensitive acid generating agents, the acid diffusion controlling agents, and the solvents constituting the radiation-sensitive resin compositions are described below.

### [Radiation-Sensitive Acid Generator]

C-1 to C-18: compounds represented by the following formulas (C-1) to (C-18)

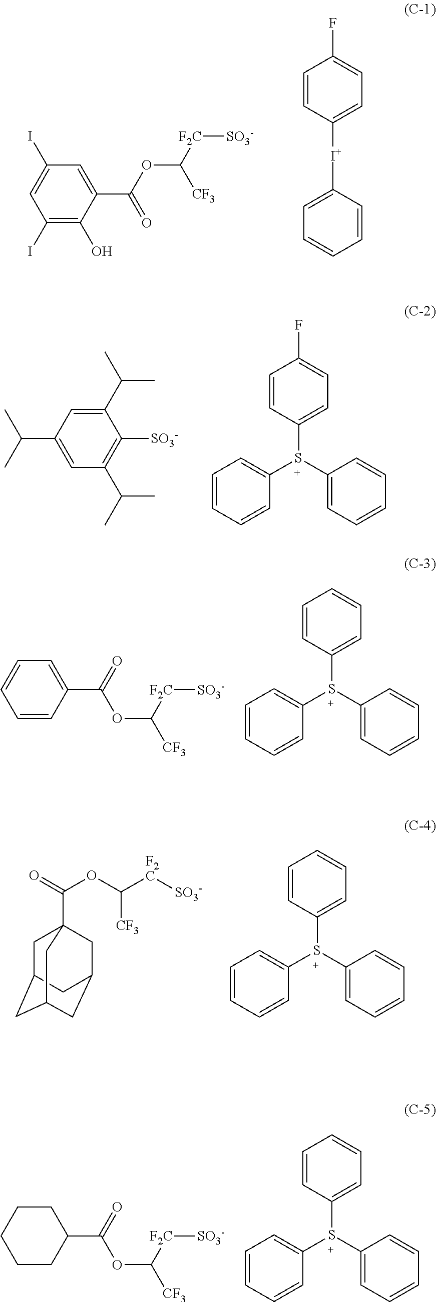

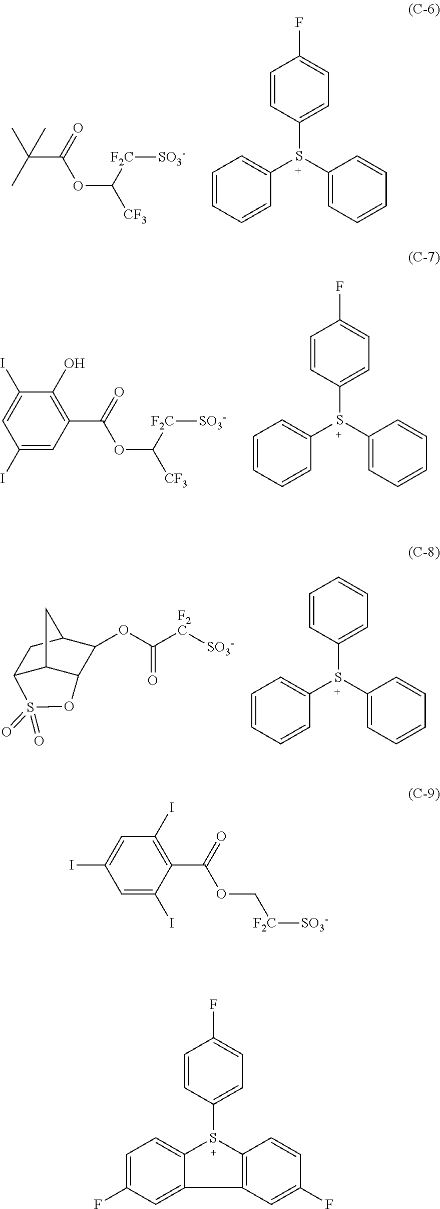

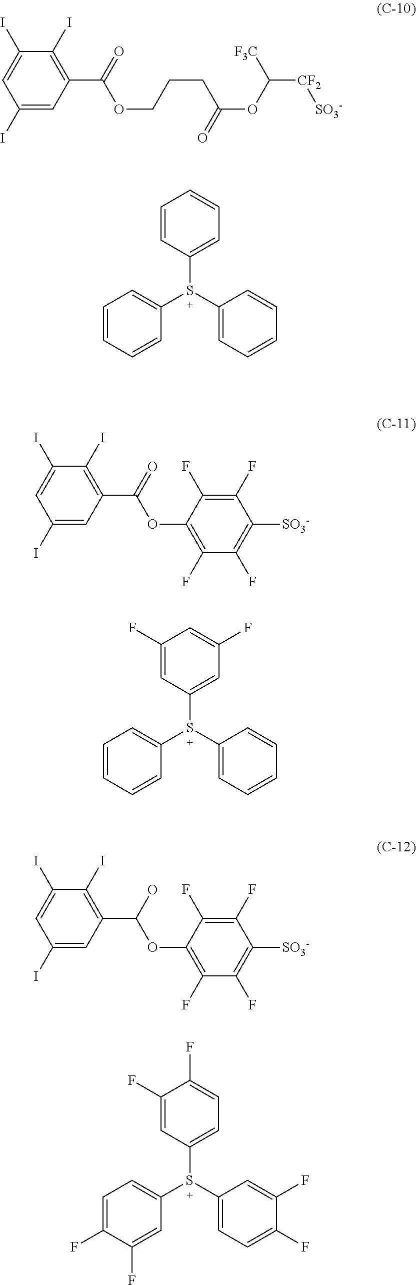

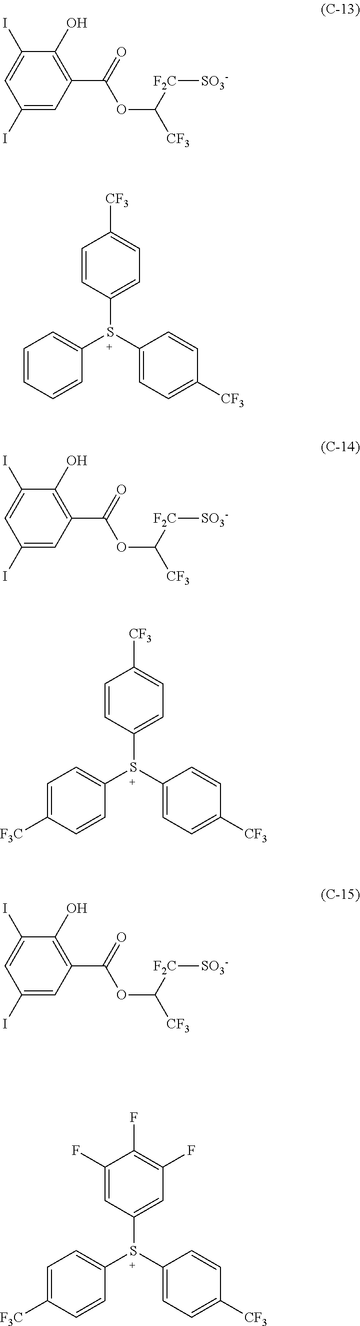

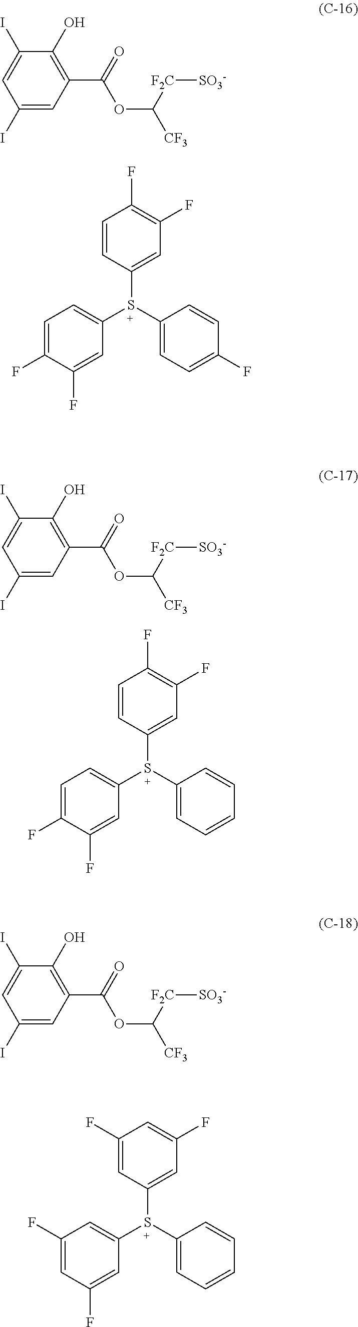

### [Acid Diffusion Controlling Agent]

D-1 to D-12: compounds represented by the following formulas (D-1) to (D-12)

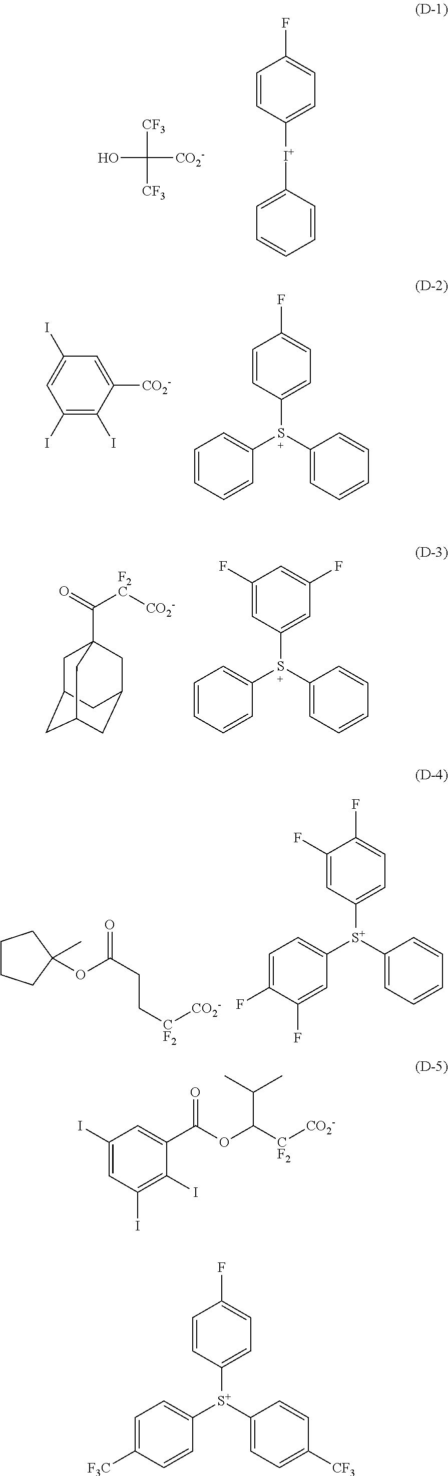

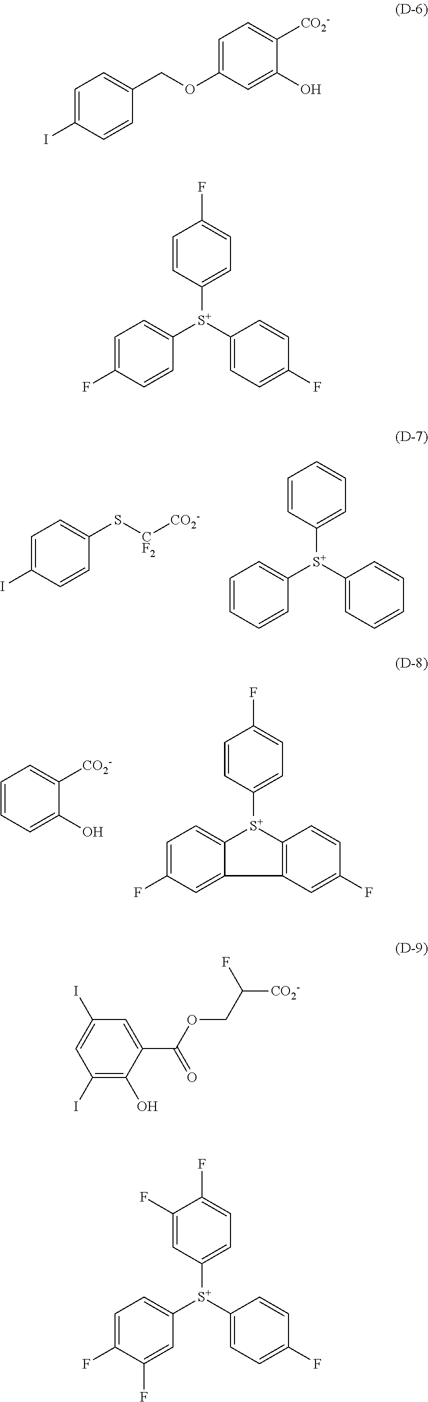

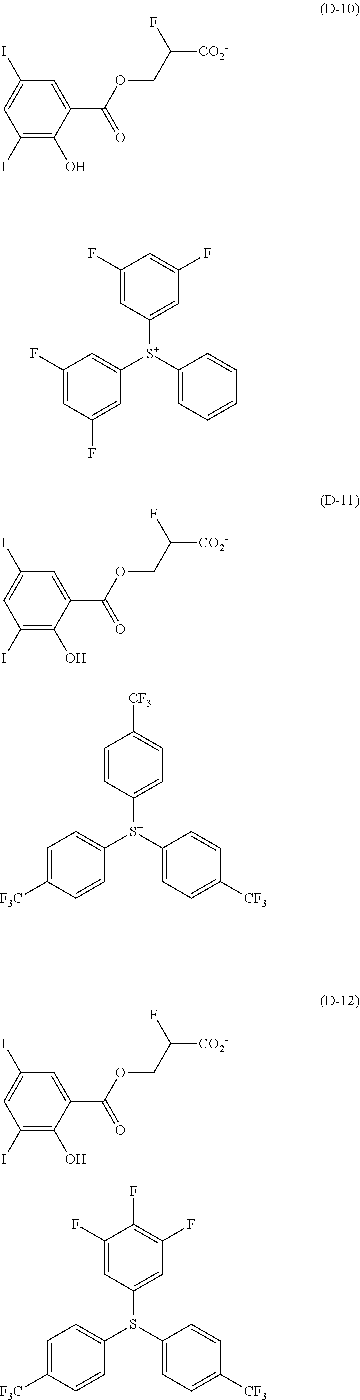

### [Solvent]

E-1: propylene glycol monomethyl ether acetate
E-2: propylene glycol-1-monomethyl ether

### Example 1

100 parts by mass of polymer (A-1), 20 parts by mass of (C-1), (D-1) in an amount of 20 mol % based on (C-1), 4,800 parts by mass of (E-1), and 2,000 parts by mass of (E-2) were blended and mixed. Next, the resulting mixed liquid was filtered through a membrane filter having a pore size of 0.20 μm to prepare a radiation-sensitive resin composition (R-1).

### Examples 2 to 99 and Comparative Examples 1 to 8

Radiation-sensitive resin compositions (R-2) to (R-99) and (CR-1) to (CR-8) were prepared in the same manner as in Example 1 except that the components of the types and the blending amounts shown in the following Tables 2 and 3 were used.

TABLE 2

| | | | | | | Acid diffusion | | | |
|---|---|---|---|---|---|---|---|---|---|
| | | | | | | controlling agent | | | |
| | Radiation- | Polymer | | Acid generator | | | mol % | Solvent | |
| | sensitive | | parts by | | parts by | | based on | | parts by |
| | composition | Type | mass | Type | mass | Type | component C | Type | mass |
| Example 1 | R-1 | A-1 | 100 | C-1 | 20 | D-1 | 20 | E-1/E-2 | 4800/2000 |
| Example 2 | R-2 | A-2 | 100 | C-1 | 20 | D-1 | 20 | E-1/E-2 | 4800/2000 |
| Example 3 | R-3 | A-3 | 100 | C-1 | 20 | D-1 | 20 | E-1/E-2 | 4800/2000 |
| Example 4 | R-4 | A-4 | 100 | C-1 | 20 | D-1 | 20 | E-1/E-2 | 4800/2000 |
| Example 5 | R-5 | A-5 | 100 | C-1 | 20 | D-1 | 20 | E-1/E-2 | 4800/2000 |
| Example 6 | R-6 | A-6 | 100 | C-1 | 20 | D-1 | 20 | E-1/E-2 | 4800/2000 |
| Example 7 | R-7 | A-7 | 100 | C-1 | 20 | D-1 | 20 | E-1/E-2 | 4800/2000 |
| Example 8 | R-8 | A-8 | 100 | C-1 | 20 | D-1 | 20 | E-1/E-2 | 4800/2000 |
| Example 9 | R-9 | A-9 | 100 | C-1 | 20 | D-1 | 20 | E-1/E-2 | 4800/2000 |
| Example 10 | R-10 | A-10 | 100 | C-1 | 20 | D-1 | 20 | E-1/E-2 | 4800/2000 |
| Example 11 | R-11 | A-11 | 100 | C-1 | 20 | D-1 | 20 | E-1/E-2 | 4800/2000 |
| Example 12 | R-12 | A-12 | 100 | C-1 | 20 | D-1 | 20 | E-1/E-2 | 4800/2000 |
| Example 13 | R-13 | A-13 | 100 | C-1 | 20 | D-1 | 20 | E-1/E-2 | 4800/2000 |
| Example 14 | R-14 | A-14 | 100 | C-1 | 20 | D-1 | 20 | E-1/E-2 | 4800/2000 |
| Example 15 | R-15 | A-15 | 100 | C-1 | 20 | D-1 | 20 | E-1/E-2 | 4800/2000 |
| Example 16 | R-16 | A-16 | 100 | C-1 | 20 | D-1 | 20 | E-1/E-2 | 4800/2000 |
| Example 17 | R-17 | A-17 | 100 | C-1 | 20 | D-1 | 20 | E-1/E-2 | 4800/2000 |
| Example 18 | R-18 | A-18 | 100 | C-1 | 20 | D-1 | 20 | E-1/E-2 | 4800/2000 |
| Example 19 | R-19 | A-19 | 100 | C-1 | 20 | D-1 | 20 | E-1/E-2 | 4800/2000 |
| Example 20 | R-20 | A-20 | 100 | C-1 | 20 | D-1 | 20 | E-1/E-2 | 4800/2000 |
| Example 21 | R-21 | A-21 | 100 | C-1 | 20 | D-1 | 20 | E-1/E-2 | 4800/2000 |
| Example 22 | R-22 | A-22 | 100 | C-1 | 20 | D-1 | 20 | E-1/E-2 | 4800/2000 |
| Example 23 | R-23 | A-23 | 100 | C-1 | 20 | D-1 | 20 | E-1/E-2 | 4800/2000 |
| Example 24 | R-24 | A-24 | 100 | C-1 | 20 | D-1 | 20 | E-1/E-2 | 4800/2000 |
| Example 25 | R-25 | A-25 | 100 | C-1 | 20 | D-1 | 20 | E-1/E-2 | 4800/2000 |
| Example 26 | R-26 | A-26 | 100 | C-1 | 20 | D-1 | 20 | E-1/E-2 | 4800/2000 |
| Example 27 | R-27 | A-27 | 100 | C-1 | 20 | D-1 | 20 | E-1/E-2 | 4800/2000 |
| Example 28 | R-28 | A-28 | 100 | C-1 | 20 | D-1 | 20 | E-1/E-2 | 4800/2000 |
| Example 29 | R-29 | A-29 | 100 | C-1 | 20 | D-1 | 20 | E-1/E-2 | 4800/2000 |
| Example 30 | R-30 | A-30 | 100 | C-1 | 20 | D-1 | 20 | E-1/E-2 | 4800/2000 |
| Example 31 | R-31 | A-31 | 100 | C-1 | 20 | D-1 | 20 | E-1/E-2 | 4800/2000 |
| Example 32 | R-32 | A-32 | 100 | C-1 | 20 | D-1 | 20 | E-1/E-2 | 4800/2000 |
| Example 33 | R-33 | A-33 | 100 | C-1 | 20 | D-1 | 20 | E-1/E-2 | 4800/2000 |
| Example 34 | R-34 | A-34 | 100 | C-1 | 20 | D-1 | 20 | E-1/E-2 | 4800/2000 |
| Example 35 | R-35 | A-35 | 100 | C-1 | 20 | D-1 | 20 | E-1/E-2 | 4800/2000 |
| Example 36 | R-36 | A-36 | 100 | C-1 | 20 | D-1 | 20 | E-1/E-2 | 4800/2000 |
| Example 37 | R-37 | A-37 | 100 | C-1 | 20 | D-1 | 20 | E-1/E-2 | 4800/2000 |
| Example 38 | R-38 | A-38 | 100 | C-1 | 20 | D-1 | 20 | E-1/E-2 | 4800/2000 |
| Example 39 | R-39 | A-39 | 100 | C-1 | 20 | D-1 | 20 | E-1/E-2 | 4800/2000 |
| Example 40 | R-40 | A-40 | 100 | C-1 | 20 | D-1 | 20 | E-1/E-2 | 4800/2000 |
| Example 41 | R-41 | A-41 | 100 | C-1 | 20 | D-1 | 20 | E-1/E-2 | 4800/2000 |
| Example 42 | R-42 | A-42 | 100 | C-1 | 20 | D-1 | 20 | E-1/E-2 | 4800/2000 |
| Example 43 | R-43 | A-43 | 100 | C-1 | 20 | D-1 | 20 | E-1/E-2 | 4800/2000 |
| Example 44 | R-44 | A-44 | 100 | C-1 | 20 | D-1 | 20 | E-1/E-2 | 4800/2000 |
| Example 45 | R-45 | A-45 | 100 | C-1 | 20 | D-1 | 20 | E-1/E-2 | 4800/2000 |
| Example 46 | R-46 | A-46 | 100 | C-1 | 20 | D-1 | 20 | E-1/E-2 | 4800/2000 |
| Example 47 | R-47 | A-47 | 100 | C-1 | 20 | D-1 | 20 | E-1/E-2 | 4800/2000 |
| Example 48 | R-48 | A-48 | 100 | C-1 | 20 | D-1 | 20 | E-1/E-2 | 4800/2000 |
| Example 49 | R-49 | A-49 | 100 | C-1 | 20 | D-1 | 20 | E-1/E-2 | 4800/2000 |
| Example 50 | R-50 | A-50 | 100 | C-1 | 20 | D-1 | 20 | E-1/E-2 | 4800/2000 |
| Example 51 | R-51 | A-51 | 100 | C-1 | 20 | D-1 | 20 | E-1/E-2 | 4800/2000 |
| Example 52 | R-52 | A-52 | 100 | C-1 | 20 | D-1 | 20 | E-1/E-2 | 4800/2000 |
| Example 53 | R-53 | A-53 | 100 | C-1 | 20 | D-1 | 20 | E-1/E-2 | 4800/2000 |
| Example 54 | R-54 | A-54 | 100 | C-1 | 20 | D-1 | 20 | E-1/E-2 | 4800/2000 |
| Example 55 | R-55 | A-55 | 100 | C-1 | 20 | D-1 | 20 | E-1/E-2 | 4800/2000 |
| Example 56 | R-56 | A-56 | 100 | C-1 | 20 | D-1 | 20 | E-1/E-2 | 4800/2000 |
| Example 57 | R-57 | A-57 | 100 | C-1 | 20 | D-1 | 20 | E-1/E-2 | 4800/2000 |
| Example 58 | R-58 | A-58 | 100 | C-1 | 20 | D-1 | 20 | E-1/E-2 | 4800/2000 |
| Example 59 | R-59 | A-59 | 100 | C-1 | 20 | D-1 | 20 | E-1/E-2 | 4800/2000 |
| Example 60 | R-60 | A-60 | 100 | C-1 | 20 | D-1 | 20 | E-1/E-2 | 4800/2000 |
| Example 61 | R-61 | A-61 | 100 | C-1 | 20 | D-1 | 20 | E-1/E-2 | 4800/2000 |
| Example 62 | R-62 | A-62 | 100 | C-1 | 20 | D-1 | 20 | E-1/E-2 | 4800/2000 |
| Example 63 | R-63 | A-63 | 100 | C-1 | 20 | D-1 | 20 | E-1/E-2 | 4800/2000 |
| Example 64 | R-64 | B-1 | 100 | C-1 | 20 | D-1 | 20 | E-1/E-2 | 4800/2000 |
| Example 65 | R-65 | A-64 | 100 | C-1 | 20 | D-1 | 20 | E-1/E-2 | 4800/2000 |
| Example 66 | R-66 | A-65 | 100 | C-1 | 20 | D-1 | 20 | E-1/E-2 | 4800/2000 |
| Example 67 | R-67 | A-66 | 100 | C-1 | 20 | D-1 | 20 | E-1/E-2 | 4800/2000 |
| Example 68 | R-68 | A-67 | 100 | C-1 | 20 | D-1 | 20 | E-1/E-2 | 4800/2000 |
| Example 69 | R-69 | A-68 | 100 | C-1 | 20 | D-1 | 20 | E-1/E-2 | 4800/2000 |
| Example 70 | R-70 | A-1 | 100 | C-1 | 30 | D-1 | 20 | E-1/E-2 | 4800/2000 |

TABLE 3

| | | | | | | Acid diffusion | | | |
|---|---|---|---|---|---|---|---|---|---|
| | | | | | | controlling agent | | | |
| | Radiation- | Resin | | Acid generator | | | mol % | Solvent | |
| | sensitive | | parts by | | parts by | | based on | | parts by |
| | composition | Type | mass | Type | mass | Type | component C | Type | mass |
| Example 71 | R-71 | A-1 | 100 | C-1 | 40 | D-1 | 20 | E-1/E-2 | 4800/2000 |
| Example 72 | R-72 | A-1 | 100 | C-2 | 20 | D-1 | 20 | E-1/E-2 | 4800/2000 |
| Example 73 | R-73 | A-1 | 100 | C-3 | 20 | D-1 | 20 | E-1/E-2 | 4800/2000 |
| Example 74 | R-74 | A-1 | 100 | C-4 | 20 | D-1 | 20 | E-1/E-2 | 4800/2000 |
| Example 75 | R-75 | A-1 | 100 | C-5 | 20 | D-1 | 20 | E-1/E-2 | 4800/2000 |
| Example 76 | R-76 | A-1 | 100 | C-6 | 20 | D-1 | 20 | E-1/E-2 | 4800/2000 |
| Example 77 | R-77 | A-1 | 100 | C-7 | 20 | D-1 | 20 | E-1/E-2 | 4800/2000 |
| Example 78 | R-78 | A-1 | 100 | C-8 | 20 | D-1 | 20 | E-1/E-2 | 4800/2000 |
| Example 79 | R-79 | A-1 | 100 | C-9 | 20 | D-1 | 20 | E-1/E-2 | 4800/2000 |
| Example 80 | R-80 | A-1 | 100 | C-10 | 20 | D-1 | 20 | E-1/E-2 | 4800/2000 |
| Example 81 | R-81 | A-1 | 100 | C-11 | 20 | D-1 | 20 | E-1/E-2 | 4800/2000 |
| Example 82 | R-82 | A-1 | 100 | C-12 | 20 | D-1 | 20 | E-1/E-2 | 4800/2000 |
| Example 83 | R-83 | A-1 | 100 | C-13 | 20 | D-1 | 20 | E-1/E-2 | 4800/2000 |
| Example 84 | R-84 | A-1 | 100 | C-14 | 20 | D-1 | 20 | E-1/E-2 | 4800/2000 |
| Example 85 | R-85 | A-1 | 100 | C-15 | 20 | D-1 | 20 | E-1/E-2 | 4800/2000 |
| Example 86 | R-86 | A-1 | 100 | C-16 | 20 | D-1 | 20 | E-1/E-2 | 4800/2000 |
| Example 87 | R-87 | A-1 | 100 | C-17 | 20 | D-1 | 20 | E-1/E-2 | 4800/2000 |
| Example 88 | R-88 | A-1 | 100 | C-18 | 20 | D-1 | 20 | E-1/E-2 | 4800/2000 |
| Example 89 | R-89 | A-1 | 100 | C-1 | 20 | D-2 | 20 | E-1/E-2 | 4800/2000 |
| Example 90 | R-90 | A-1 | 100 | C-1 | 20 | D-3 | 20 | E-1/E-2 | 4800/2000 |
| Example 91 | R-91 | A-1 | 100 | C-1 | 20 | D-4 | 20 | E-1/E-2 | 4800/2000 |
| Example 92 | R-92 | A-1 | 100 | C-1 | 20 | D-5 | 20 | E-1/E-2 | 4800/2000 |
| Example 93 | R-93 | A-1 | 100 | C-1 | 20 | D-6 | 20 | E-1/E-2 | 4800/2000 |
| Example 94 | R-94 | A-1 | 100 | C-1 | 20 | D-7 | 20 | E-1/E-2 | 4800/2000 |
| Example 95 | R-95 | A-1 | 100 | C-1 | 20 | D-8 | 20 | E-1/E-2 | 4800/2000 |
| Example 96 | R-96 | A-1 | 100 | C-1 | 20 | D-9 | 20 | E-1/E-2 | 4800/2000 |
| Example 97 | R-97 | A-1 | 100 | C-1 | 20 | D-10 | 20 | E-1/E-2 | 4800/2000 |
| Example 98 | R-98 | A-1 | 100 | C-1 | 20 | D-11 | 20 | E-1/E-2 | 4800/2000 |
| Example 99 | R-99 | A-1 | 100 | C-1 | 20 | D-12 | 20 | E-1/E-2 | 4800/2000 |
| Comparative | CR-1 | A-69 | 100 | C-1 | 20 | D-1 | 20 | E-1/E-2 | 4800/2000 |
| Example 1 | | | | | | | | | |
| Comparative | CR-2 | A-70 | 100 | C-1 | 20 | D-1 | 20 | E-1/E-2 | 4800/2000 |
| Example 2 | | | | | | | | | |
| Comparative | CR-3 | A-71 | 100 | C-1 | 20 | D-1 | 20 | E-1/E-2 | 4800/2000 |
| Example 3 | | | | | | | | | |
| Comparative | CR-4 | A-72 | 100 | C-1 | 20 | D-1 | 20 | E-1/E-2 | 4800/2000 |
| Example 4 | | | | | | | | | |
| Comparative | CR-5 | A-73 | 100 | C-1 | 20 | D-1 | 20 | E-1/E-2 | 4800/2000 |
| Example 5 | | | | | | | | | |
| Comparative | CR-6 | A-74 | 100 | C-1 | 20 | D-1 | 20 | E-1/E-2 | 4800/2000 |
| Example 6 | | | | | | | | | |
| Comparative | CR-7 | A-75 | 100 | C-1 | 20 | D-1 | 20 | E-1/E-2 | 4800/2000 |
| Example 7 | | | | | | | | | |
| Comparative | CR-8 | A-76 | 100 | C-1 | 20 | D-1 | 20 | E-1/E-2 | 4800/2000 |
| Example 8 | | | | | | | | | |

### <Formation of Resist Pattern> (EUV Exposure, Alkaline Development)

Each of the radiation-sensitive resin compositions prepared as described above was applied using a spin coater (CLEAN TRACK ACT12, manufactured by Tokyo Electron Ltd.) to a surface of a 12-inch silicon wafer with a 20 nm thick underlayer film (AL412, manufactured by Brewer Science). Soft baking (SB) was performed at 130° C. for 60 seconds, followed by cooling at 23° C. for 30 seconds, and thus a resist film having a thickness of 50 nm was formed.

Then, the resist film was irradiated with EUV light using an EUV scanner (type “NXE3300”, manufactured by ASML, NA=0.33, lighting condition: Conventional, s=0.89, Mask imecDEFECT32FFR02). Next, post exposure baking (PEB) was performed at 90° C. for 60 seconds, followed by development at 23° C. for 30 seconds with a 2.38 wt % aqueous TMAH solution, and thus a positive 32 nm line-and-space pattern was formed.

### <Evaluation>

The LWR performance and process window of each of the radiation-sensitive resin compositions were evaluated by measuring the resist patterns formed above according to the following method. A scanning electron microscope (“CG-4100” manufactured by Hitachi High-Tech Corporation) was used for measuring the length of the resist pattern. The evaluation results are shown in the following Tables 4 and 5.

### [Sensitivity]

An exposure amount at which a 32 nm line-and-space pattern was formed in the formation of the resist pattern was defined as an optimum exposure amount, and the optimum exposure amount was defined as sensitivity (mJ/cm²).

### [LWR Performance]

The resist pattern was observed from above using the scanning electron microscope. Line widths were measured at a total of 50 optional points. A 3 sigma value was obtained from the distribution of the measurement values, and defined as LWR performance. The smaller the value is, the better the LWR performance is.

### [Process Window (CD Margin Performance)]

A pattern was formed at a low exposure amount to a high exposure amount using a mask forming a 32 nm line-and-space (1L/1S). In general, connection between patterns is observed on a low exposure amount side, and defects such as pattern collapse are observed on a high exposure amount side. The difference between the upper limit value and the lower limit value of the resist dimension in which these defects were not observed was defined as a “CD margin”. It is considered that the larger the value of the CD margin is, the wider the process window is.

TABLE 4

| | Radiation- | Eop | | CD |
|---|---|---|---|---|
| | sensitive | (mJ/ | LWR | margin |
| | composition | cm²) | (nm) | (nm) |
| Example 1 | R-1 | 27 | 3.5 | 36 |
| Example 2 | R-2 | 26 | 3.7 | 37 |
| Example 3 | R-3 | 24 | 3.8 | 38 |
| Example 4 | R-4 | 25 | 3.6 | 31 |
| Example 5 | R-5 | 24 | 3.5 | 35 |
| Example 6 | R-6 | 24 | 3.3 | 38 |
| Example 7 | R-7 | 23 | 3.4 | 37 |
| Example 8 | R-8 | 24 | 3.3 | 38 |
| Example 9 | R-9 | 23 | 3.5 | 37 |
| Example 10 | R-10 | 27 | 3.5 | 36 |
| Example 11 | R-11 | 23 | 3.4 | 35 |
| Example 12 | R-12 | 23 | 3.5 | 35 |
| Example 13 | R-13 | 27 | 3.3 | 34 |
| Example 14 | R-14 | 26 | 3.5 | 36 |
| Example 15 | R-15 | 26 | 3.7 | 35 |
| Example 16 | R-16 | 25 | 3.3 | 35 |
| Example 17 | R-17 | 24 | 3.4 | 36 |
| Example 18 | R-18 | 26 | 3.5 | 34 |
| Example 19 | R-19 | 24 | 3.5 | 34 |
| Example 20 | R-20 | 22 | 3.6 | 34 |
| Example 21 | R-21 | 26 | 3.7 | 30 |
| Example 22 | R-22 | 27 | 3.6 | 33 |
| Example 23 | R-23 | 30 | 3.9 | 31 |
| Example 24 | R-24 | 29 | 3.9 | 30 |
| Example 25 | R-25 | 29 | 3.8 | 35 |
| Example 26 | R-26 | 25 | 3.5 | 36 |
| Example 27 | R-27 | 25 | 3.4 | 36 |
| Example 28 | R-28 | 24 | 3.5 | 36 |
| Example 29 | R-29 | 22 | 3.7 | 34 |
| Example 30 | R-30 | 21 | 3.9 | 30 |
| Example 31 | R-31 | 24 | 3.8 | 33 |
| Example 32 | R-32 | 24 | 3.7 | 31 |
| Example 33 | R-33 | 22 | 3.6 | 32 |
| Example 34 | R-34 | 21 | 3.7 | 35 |
| Example 35 | R-35 | 30 | 3.3 | 37 |
| Example 36 | R-36 | 29 | 3.4 | 32 |
| Example 37 | R-37 | 24 | 3.5 | 38 |
| Example 38 | R-38 | 25 | 3.7 | 35 |
| Example 39 | R-39 | 30 | 3.9 | 32 |
| Example 40 | R-40 | 26 | 3.8 | 32 |
| Example 41 | R-41 | 29 | 3.5 | 35 |
| Example 42 | R-42 | 29 | 3.3 | 37 |
| Example 43 | R-43 | 23 | 3.7 | 32 |
| Example 44 | R-44 | 22 | 3.8 | 36 |
| Example 45 | R-45 | 28 | 3.7 | 35 |
| Example 46 | R-46 | 27 | 3.8 | 35 |
| Example 47 | R-47 | 26 | 3.6 | 31 |
| Example 48 | R-48 | 25 | 3.7 | 32 |
| Example 49 | R-49 | 23 | 3.9 | 35 |
| Example 50 | R-50 | 22 | 3.8 | 37 |
| Example 51 | R-51 | 24 | 3.5 | 35 |
| Example 52 | R-52 | 24 | 3.8 | 38 |
| Example 53 | R-53 | 22 | 3.6 | 35 |
| Example 54 | R-54 | 23 | 3.5 | 36 |
| Example 55 | R-55 | 27 | 3.8 | 33 |
| Example 56 | R-56 | 28 | 3.8 | 31 |
| Example 57 | R-57 | 29 | 3.7 | 33 |
| Example 58 | R-58 | 30 | 3.6 | 34 |
| Example 59 | R-59 | 29 | 3.8 | 32 |
| Example 60 | R-60 | 29 | 3.8 | 33 |
| Example 61 | R-61 | 30 | 3.7 | 31 |
| Example 62 | R-62 | 28 | 3.8 | 33 |
| Example 63 | R-63 | 30 | 3.9 | 34 |
| Example 64 | R-64 | 30 | 3.8 | 32 |
| Example 65 | R-65 | 31 | 3.7 | 30 |
| Example 66 | R-66 | 30 | 3.8 | 30 |
| Example 67 | R-67 | 28 | 3.6 | 34 |
| Example 68 | R-68 | 28 | 3.5 | 35 |
| Example 69 | R-69 | 27 | 3.4 | 36 |
| Example 70 | R-70 | 24 | 3.7 | 35 |

TABLE 5

| | Radiation- | Eop | | CD |
|---|---|---|---|---|
| | sensitive | (mJ/ | LWR | margin |
| | composition | cm²) | (nm) | (nm) |
| Example 71 | R-71 | 22 | 3.7 | 34 |
| Example 72 | R-72 | 28 | 3.7 | 36 |
| Example 73 | R-73 | 30 | 3.8 | 32 |
| Example 74 | R-74 | 25 | 3.9 | 33 |
| Example 75 | R-75 | 23 | 3.7 | 31 |
| Example 76 | R-76 | 29 | 3.5 | 34 |
| Example 77 | R-77 | 25 | 3.8 | 33 |
| Example 78 | R-78 | 28 | 3.7 | 31 |
| Example 79 | R-79 | 28 | 3.6 | 30 |
| Example 80 | R-80 | 27 | 3.6 | 35 |
| Example 81 | R-81 | 24 | 3.9 | 34 |
| Example 82 | R-82 | 29 | 3.8 | 31 |
| Example 83 | R-83 | 26 | 3.4 | 30 |
| Example 84 | R-84 | 28 | 3.8 | 36 |
| Example 85 | R-85 | 29 | 3.7 | 32 |
| Example 86 | R-86 | 29 | 3.9 | 32 |
| Example 87 | R-87 | 26 | 3.6 | 35 |
| Example 88 | R-88 | 28 | 3.8 | 33 |
| Example 89 | R-89 | 28 | 3.5 | 31 |
| Example 90 | R-90 | 27 | 3.9 | 36 |
| Example 91 | R-91 | 26 | 3.7 | 30 |
| Example 92 | R-92 | 28 | 3.9 | 37 |
| Example 93 | R-93 | 27 | 3.9 | 31 |
| Example 94 | R-94 | 26 | 3.7 | 32 |
| Example 95 | R-95 | 30 | 3.5 | 35 |
| Example 96 | R-96 | 29 | 3.6 | 30 |
| Example 97 | R-97 | 27 | 3.9 | 31 |
| Example 98 | R-98 | 27 | 3.8 | 32 |
| Example 99 | R-99 | 28 | 3.8 | 31 |
| Comparative | CR-1 | 27 | 4.4 | 31 |
| Example 1 | | | | |
| Comparative | CR-2 | 27 | 4.3 | 29 |
| Example 2 | | | | |
| Comparative | CR-3 | 26 | 4.4 | 28 |
| Example 3 | | | | |
| Comparative | CR-4 | 34 | 4.5 | 27 |
| Example 4 | | | | |
| Comparative | CR-5 | 24 | 4.7 | 29 |
| Example 5 | | | | |
| Comparative | CR-6 | 38 | 4.2 | 28 |
| Example 6 | | | | |
| Comparative | CR-7 | 20 | 4.9 | 28 |
| Example 7 | | | | |
| Comparative | CR-8 | 27 | 4.2 | 27 |
| Example 8 | | | | |

According to the radiation-sensitive resin composition and the method for forming a resist pattern of the present invention, sensitivity, sensitivity, LWR performance, and CD margin performance can be improved as compared with the conventional technology. Therefore, they can be suitably used for the formation of a fine resist pattern in a lithography process in the manufacture of various electronic devices such as semiconductor devices and liquid crystal devices.

Obviously, numerous modifications and variations of the present invention(s) are possible in light of the above teachings. It is therefore to be understood that within the scope of the appended claims, the invention(s) may be practiced otherwise than as specifically described herein.

What is claimed is:

1. A radiation-sensitive resin composition comprising:
a resin comprising a structural unit represented by formula (1);
a radiation-sensitive acid generator; and
a solvent: 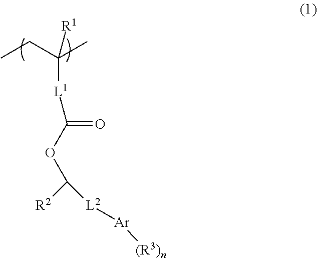
wherein, in the formula (1):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
Ar represents a group obtained by removing (n+1) hydrogen atoms from an aromatic ring;
n is an integer of 1 or more;
when n is 1, R³ is a halogen atom or a halogenated hydrocarbon group; and
when n is 2 or more, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.

2. The radiation-sensitive resin composition according to claim 1, wherein the structural unit represented by the formula (1) is represented by formula (2) or (3): 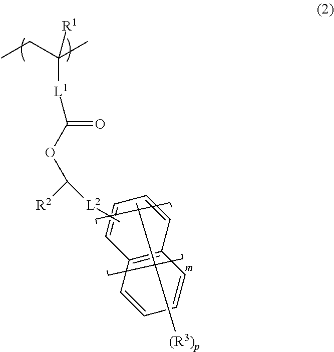
wherein, in the formula (2):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
m is 0 or 1;
p is an integer of 1 to (5+2m);
when p is 1, R³ is a halogen atom or a halogenated hydrocarbon group; and
when p is an integer of 2 to (5+2m), each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group, 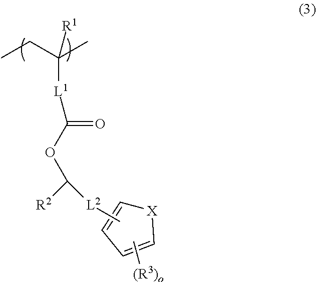
wherein, in the formula (3):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
X represents an oxygen atom, —NH—, or a sulfur atom;
o is an integer of 1 to 3;
when o is 1, R³ is a halogen atom or a halogenated hydrocarbon group; and
when o is 2 or 3, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.

3. The radiation-sensitive resin composition according to claim 1, wherein L² is a single bond or a divalent hydrocarbon group having 1 to 10 carbon atoms.

4. The radiation-sensitive resin composition according to claim 1, further comprising a photodegradable base.

5. The radiation-sensitive resin composition according to claim 1, wherein the resin further comprises a structural unit represented by formula (4): 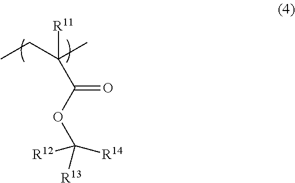
wherein in the formula (4):
R¹¹ is a hydrogen atom, a fluorine atom, a methyl group, or a trifluoromethyl group;
R¹² is a monovalent hydrocarbon group having 1 to 20 carbon atoms; and
R¹³ and R¹⁴ each independently represent a monovalent chain hydrocarbon group having 1 to 10 carbon atoms or a monovalent alicyclic hydrocarbon group having 3 to 20 carbon atoms, and optionally R¹³ and R¹⁴ combines to form an alicyclic hydrocarbon group having 3 to 20 carbon atoms, together with the carbon atom to which R¹³ and R¹⁴ are bonded.

6. The radiation-sensitive resin composition according to claim 1, wherein the resin further comprises a structural unit having a phenolic hydroxy group.

7. The radiation-sensitive resin composition according to claim 1, wherein the resin further comprises a structural unit comprising a lactone structure, a cyclic carbonate structure, a sultone structure, or combinations thereof.

8. The radiation-sensitive resin composition according to claim 1, wherein n is an integer of 1 to 3.

9. The radiation-sensitive resin composition according to claim 1, wherein an amount of the structural unit represented by the formula (1) in the resin is 1 to 70 mol % relative to all structural units constituting the resin.

10. A method of forming a pattern, the method comprising:
directly or indirectly applying the radiation-sensitive resin composition according to claim 1 to a substrate to form a resist film;
exposing the resist film to light; and
developing the exposed resist film.

11. The method according to claim 10, wherein the light to which the resist film is exposed is an extreme ultraviolet ray (EUV), an X-ray, or an electron beam (EB).

12. A polymer comprising a structural unit represented by formula (2) or formula (3): 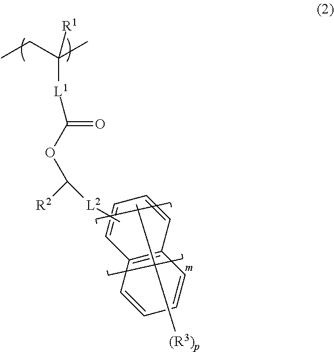
wherein, in the formula (2):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
m is 0 or 1;
p is an integer of 1 to (5+2m);
when p is 1, R³ is a halogen atom or a halogenated hydrocarbon group, and
when p is an integer of 2 to (2+2m), each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group, 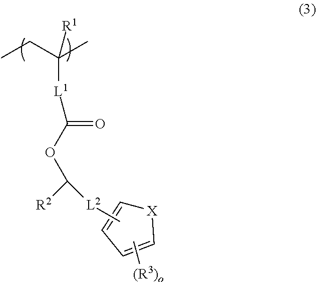
wherein, in the formula (3):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
X represents an oxygen atom, —NH—, or a sulfur atom;
o is an integer of 1 to 3;
when o is 1, R³ is a halogen atom or a halogenated hydrocarbon group; and
when o is 2 or 3, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms; at least one R³ is a halogen atom or a halogenated hydrocarbon group, a plurality of R³'s are same or different from each other, and at least one R³ is a halogen atom or a halogenated hydrocarbon group.

13. A compound represented by formula (5) or formula (6): 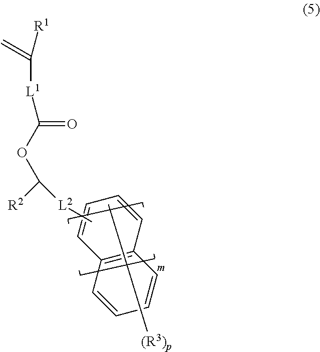
wherein, in the formula (5):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
m is 0 or 1;
p is an integer of 1 to (5+2m);
when p is 1, R³ is a halogen atom, a halogenated hydrocarbon group; and
when p is an integer of 2 to (5+2m), each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group, 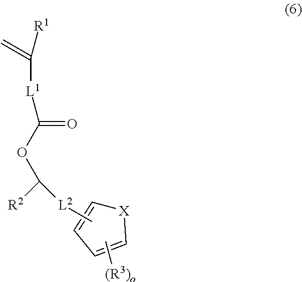
wherein, in the formula (6):
R¹ is a hydrogen atom, a fluorine atom, a methyl group, or a trifluoromethyl group;
L¹ represents a single bond or —COO-L-;
L represents a substituted or unsubstituted alkanediyl group;
R² represents a monovalent hydrocarbon group having 1 to 20 carbon atoms;
L² represents a single bond or a divalent linking group;
X represents an oxygen atom, —NH—, or a sulfur atom;
o is an integer of 1 to 3;
when o is 1, R³ is a halogen atom or a halogenated hydrocarbon group; and
when o is 2 or 3, each R³ is independently a halogen atom, a halogenated hydrocarbon group, a hydroxy group, a monovalent hydrocarbon group having 1 to 10 carbon atoms, or a monovalent alkyl ether group having 1 to 10 carbon atoms, a plurality of R³'s are same or different from each other, and at least one R³ is a halogen atom or a halogenated hydrocarbon group.
